(19) United States
(12) Patent Application Publication
(10) Pub. No.: US 2022/0315632 A1
(43) Pub. Date: Oct. 6, 2022

(54) COMBINATION OF DIETARY PEPTIDES

(71) Applicant: DIET4LIFE APS, Hammel (DK)

(72) Inventors: Jan Stagsted, Hammel (DK); Jiehui Zhou, Brabrand (DK); Randi Jessen, Trige (DK)

(21) Appl. No.: 17/438,772

(22) PCT Filed: Mar. 13, 2020
(86) PCT No.: PCT/EP2020/056920

§ 371 Date: Sep. 13, 2021

(30) Foreign Application Priority Data
Mar. 15, 2019 (EP) 19163078.9

(51) Int. Cl. C07K 14/47; A61P 3/04

(52) U.S. Cl. CPC C07K 14/4716; A61P 3/04; A23L 33/18

(57) ABSTRACT

The present invention relates to the combination of dietary peptides, composition comprising such combinations including nutritional supplements and methods for inducing satiation and satiety, for preventing or reducing the incidence of metabolic syndrome comprising overweight and obesity, cardiovascular diseases, atherosclerosis, hypertension, hepatosteatosis, diabetes and/or cancer.

### CROSS-REFERENCE TO RELATED APPLICATIONS

This application is a national stage filing under 35 U.S.C. 371 of PCT/EP2020/056920 filed Mar. 13, 2020, which was published by the International Bureau in English on Sep. 24, 2020, and which claims priority from European Application No. 19163078.9, filed Mar. 15, 2019, each of which is hereby incorporated in its entirety by reference in this application.

### REFERENCE TO SEQUENCE LISTING SUBMITTED ELECTRONICALLY

The official copy of the sequence listing is submitted electronically via EFS-Web as an ASCII formatted sequence listing with a file named 21950PCT00-TPTO-seqlist.txt, created on Sep. 13, 2021, and having a size of 192,976 bytes, which is identical to the sequence listing submitted for International Application No. PCT/EP2020/056920 on Mar. 13, 2020. The sequence listing contained in this ASCII formatted document is part of the specification and is herein incorporated by reference in its entirety.

### FIELD OF THE INVENTION

The present invention relates to the combination of dietary peptides, composition comprising such combinations including nutritional supplements and methods for inducing satiation and satiety, for preventing or reducing the incidence of metabolic syndrome comprising overweight and obesity, cardiovascular diseases, atherosclerosis, hypertension, hepatosteatosis, diabetes and/or cancer.

### BACKGROUND OF THE INVENTION

Obesity is a common medical condition affecting numerous humans throughout the world and is associated with, induces or increases the risk of developing conditions such as cardiovascular diseases, atherosclerosis, hypertension, hepatosteatosis, cancer and/or diabetes.

Some regulators of obesity have been identified. However, despite intensive study, the regulation of obesity is still poorly understood.

Protein is more satiating than carbohydrate and fat, and its effect on food intake is more than can be accounted for by its energy content alone. The mechanism by which proteins trigger food intake regulatory systems is unclear. However, it seems likely that satiety signals arising from protein ingestion begin in the gastrointestinal tract upon proteolytic digestion.

Accordingly, dietary proteolytic products (peptides and amino acids) induce signalling in enteroendocrine cells of the intestine, which leads to secretion of various gut hormones, e.g. glucagon-like peptide-1 (GLP-1) with neuronal, local (auto- and paracrine) and systemic (endocrine) effects, ultimately leading to satiation (amount of food ingested as a meal) and satiety (length of time between meals). It is well-known that (some) enteroendocrine cells respond to free amino acids and small peptides (di- and tripeptides), which are readily taken up by the enterocytes and metabolized and/or transported into systemic circulation. Rate of digestion, i.e. transit time in the GI tract, secretion of digestive enzymes, etc, is a highly regulated process, where cellular responses to undigested proteins and/or increases in amino acids and peptides in the gut leads to secretion of gut hormones, e.g. GLP-1, peptide tyrosine-tyrosine (PYY), neurotensin (NT), which induces satiation. If these signals persist in the gut because of slow and prolonged release, satiety is enhanced. One such mechanism is the ileal brake, where unknown components in partly digested food reaches the distal small intestine and invokes a response in the form of secretion of the gut hormones GLP-1, PYY, NT and possibly others, as yet unknown hormones. However, the precise mechanism behind the ileal brake is unknown.

Glucagon-like peptide-1 (GLP-1) and glucose-dependent insulinotropic peptide (GIP) are gut hormones called incretins and are involved in glucose homeostasis by eliciting insulin secretion from pancreatic β-cells. Both incretins have been shown to play a role in diabetes and furthermore to act as anorexigenic peptides that delays gastric emptying and glucagon secretion. GLP-1 is released by L-cells after nutrient absorption. GLP-17-36 is the most abundant form in the circulation and is a potent insulinotropic peptide, which is mediated by GLP-1 receptor (GLP-1R).

The specific peptide(s) responsible for this satiety inducing signal(s) and how these peptide hormones work together in a complex system is largely unknown and it would be of great importance if better combinations of any of these peptides could be identified.

### OBJECT OF THE INVENTION

It is an object of embodiments of the invention to provide improved combinations of polypeptides that induce or signals satiety in a subject.

The combination of polypeptides according to the invention may be used to treat conditions associated with a wide variety of metabolic diseases, for use in weight management, and/or for preventing or reducing the incidence of overweight and/or obesity, or for preventing or reducing cardiovascular diseases, atherosclerosis, hypertension, hepatosteatosis, cancer and/or diabetes.

### SUMMARY OF THE INVENTION

Dietary proteolytic products (peptides and amino acids) induce signaling in enteroendocrine cells of the intestine, which leads to secretion of various gut hormones, e.g. GLP-1 with both central (CNS), local (auto- and paracrine) and systemic (endocrine) effects, ultimately leading to satiation and satiety.

It has been found by the present inventor(s) that novel meat-derived polypeptides are superior in signalling of intestinal cell lines and that only very specific peptides are capable of signalling. The inventors of the present invention have identified polypeptides including an octapeptide (ASDKPYIL, SEQ ID NO:6) present in proteolytic digests (FIG. 5) and resistant to pepsin degradation, of which a pentapeptide (KPYIL, SEQ ID NO:9) is the minimal sequence with significant biologic activity (FIG. 6). The octapeptide sequence is unique for the muscle-specific alpha-actinin-2 protein, and the sequence is conserved between all animal species. This peptide would be applicable as a novel, but natural nutritional supplement to induce satiation and satiety.

Also, previous studies have shown an effect of GLP-1 or a GLP-1 analogue, liraglutide, on suppressed feed intake, loss of body weight and reduced gastric emptying. Accordingly, the present inventors expected that a combination of certain specific dietary peptides with GLP-1 or a GLP-1 analogue would have an additive effect on gastric emptying.

So, in a first aspect the present invention relates to a combination of
1) a first polypeptide comprising the amino acid sequence
AA1-AA2-AA3-K-AA5-AA6-AA7-AA8 (formula I; SEQ ID NO:1),

wherein AA1 when present is an amino acid selected from A, L, I, and V; AA2 when present is an amino acid selected from S, T, G, A, N, E and D; AA3 when present is an amino acid selected from D, E, and G; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and
2) a second peptide hormone, such as a polypeptide being a GLP-1 receptor agonist.

In a second aspect the present invention relates to a composition comprising a combination according to the invention.

In a third aspect the present invention relates to a combination according to the invention for use in promoting satiety or for reducing feed intake in a subject.

In a fourth aspect the present invention relates to a combination according to the invention for use in weight management, and/or for preventing or reducing the incidence of overweight and/or obesity in a subject.

In a further aspect the present invention relates to a combination according to the invention for use in preventing or reducing cardiovascular diseases, atherosclerosis, hypertension, hepatosteatosis, cancer and/or diabetes.

In a further aspect the present invention relates to a method of promoting satiety or for reducing feed intake in a subject, comprising enteral administering to a subject in need thereof a combination of
1) a first polypeptide comprising or consisting of the amino acid sequence
AA1-AA2-AA3-AA4-AA5-AA6-AA7-AA8 (formula III, SEQ ID NO:3),

wherein AA1 when present is an amino acid selected from A, L, I, and V; AA2 when present is an amino acid selected from S, T, G, A, N, E and D; AA3 when present is an amino acid selected from D, R, K, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and

2) a second peptide hormone, such as a polypeptide being a GLP-1 receptor agonist.

In a further aspect the present invention relates to a method of preventing or reducing the incidence of obesity in a subject, comprising enteral administering to a subject in need thereof a combination of
1) a first polypeptide comprising or consisting of the amino acid sequence
AA1-AA2-AA3-AA4-AA5-AA6-AA7-AA8 (formula III, SEQ ID NO:3),

wherein AA1 when present is an amino acid selected from A, L, I, and V; AA2 when present is an amino acid selected from S, T, G, A, N, E and D; AA3 when present is an amino acid selected from D, R, K, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and
2) a second peptide hormone, such as a polypeptide being a GLP-1 receptor agonist.

In a further aspect the present invention relates to a method to reduce or treat cardiovascular diseases, atherosclerosis, hypertension, hepatosteatosis, cancer and/or diabetes comprising enteral administering to a subject in need thereof a combination of
1) a first polypeptide comprising or consisting of the amino acid sequence
AA1-AA2-AA3-AA4-AA5-AA6-AA7-AA8 (formula III, SEQ ID NO:3),

wherein AA1 when present is an amino acid selected from A, L, I, and V; AA2 when present is an amino acid selected from S, T, G, A, N, E and D; AA3 when present is an amino acid selected from D, R, K, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and
2) a second peptide hormone, such as a polypeptide being a GLP-1 receptor agonist.

In a further aspect the present invention relates to a method of promoting satiety or for reducing feed intake in a subject, comprising administering to a subject in need thereof a composition according to the invention.

In a further aspect the present invention relates to a method of preventing or reducing the incidence of obesity in a subject, comprising administering to a subject in need thereof a composition according to the invention.

In a further aspect the present invention relates to a method to reduce or treat cardiovascular diseases, atherosclerosis, hypertension, hepatosteatosis, cancer and/or diabetes comprising administering to a subject in need thereof a composition according to the invention.

### LEGENDS TO THE FIGURES

FIG. 1. Verification of identified sequence ASDKPYIL by synthetic peptide. Comparison of dose-response relationship of meat hydrolysate and pure, synthetic peptide identified by sequencing of purified fractions.

FIG. 2. Identification of minimal active sequence in ASDKPYIL in murine (mIC) and human (hIC) intestinal cells.

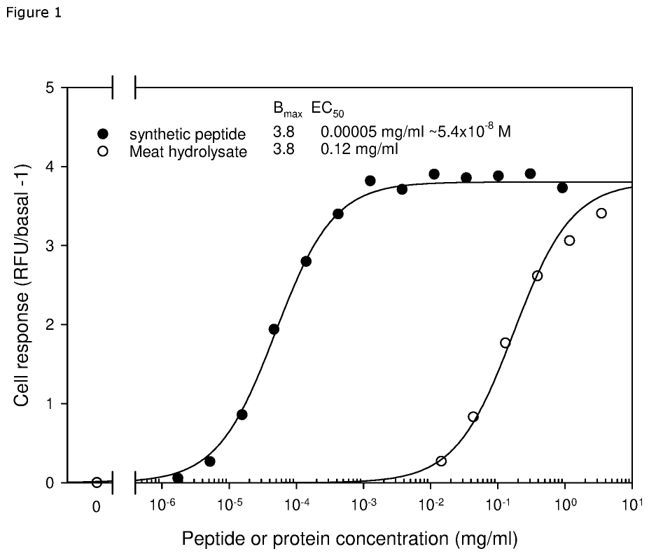

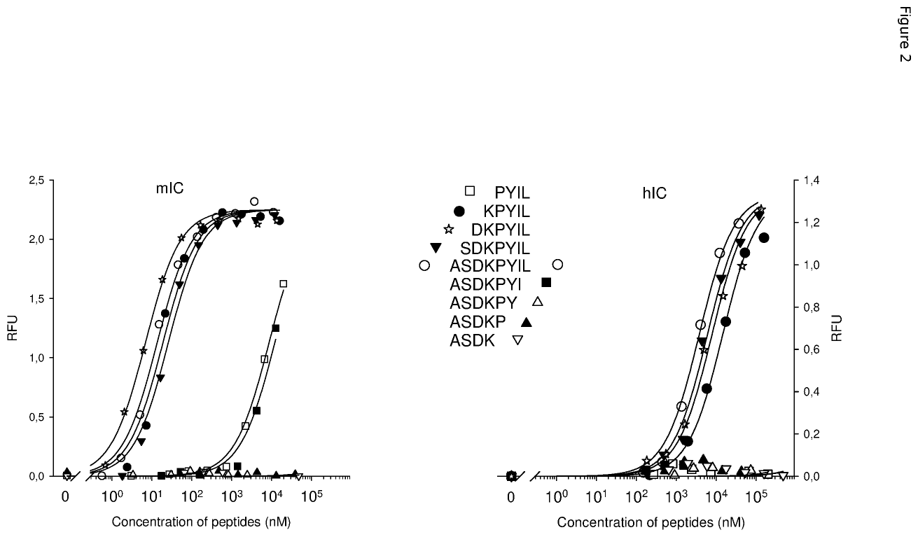

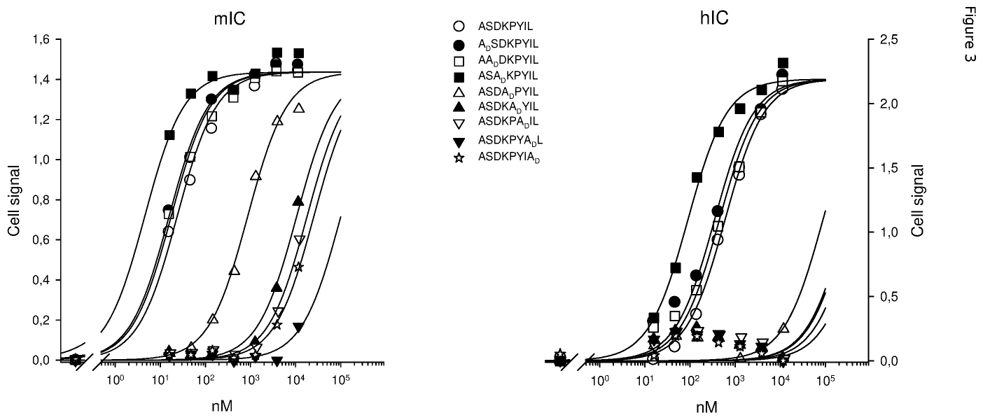

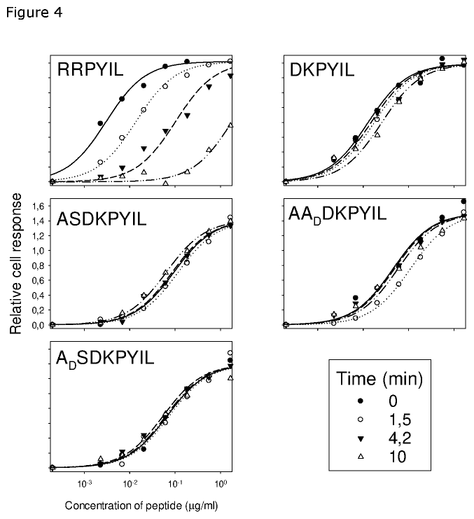

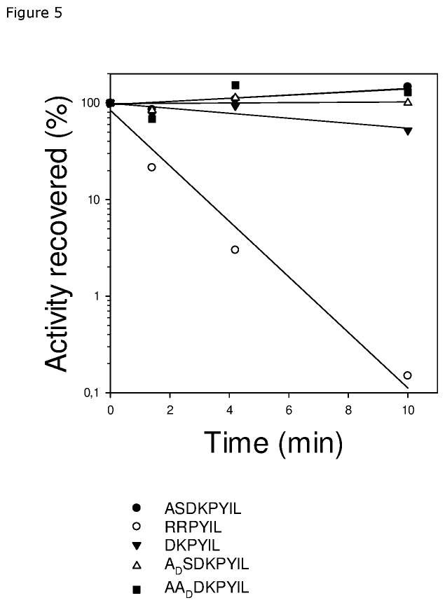

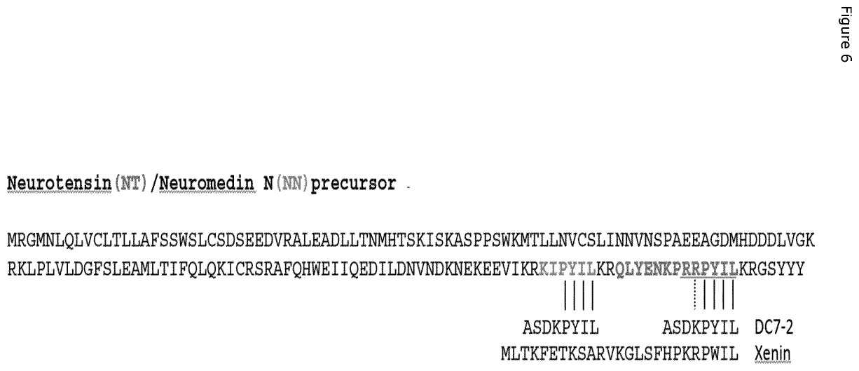

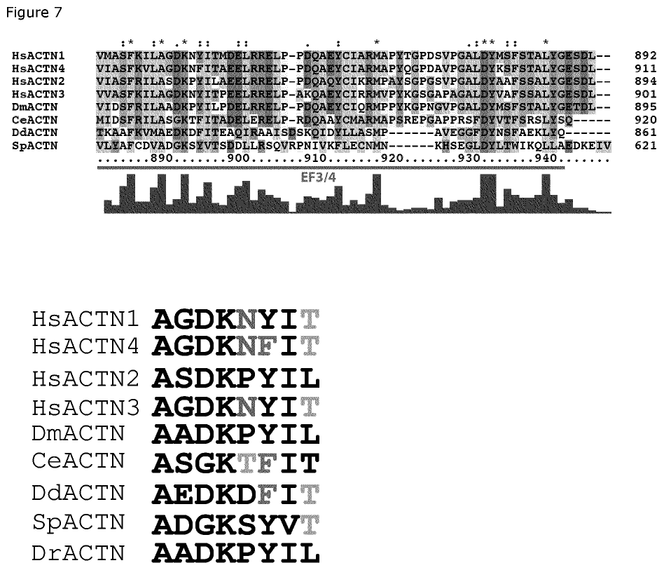

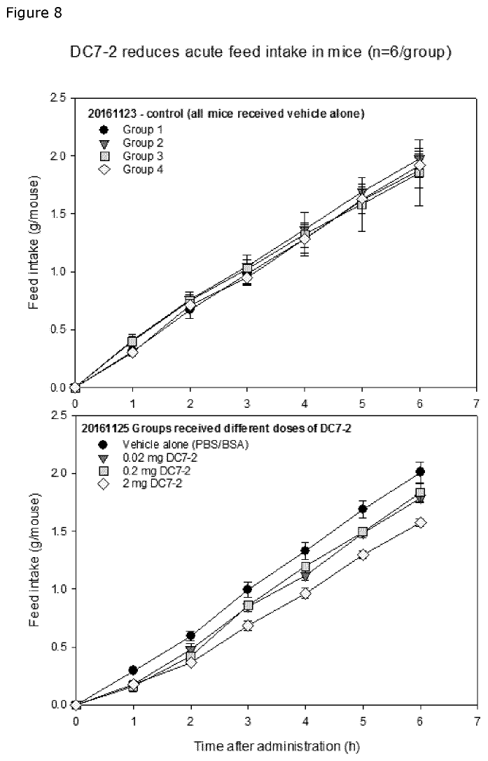

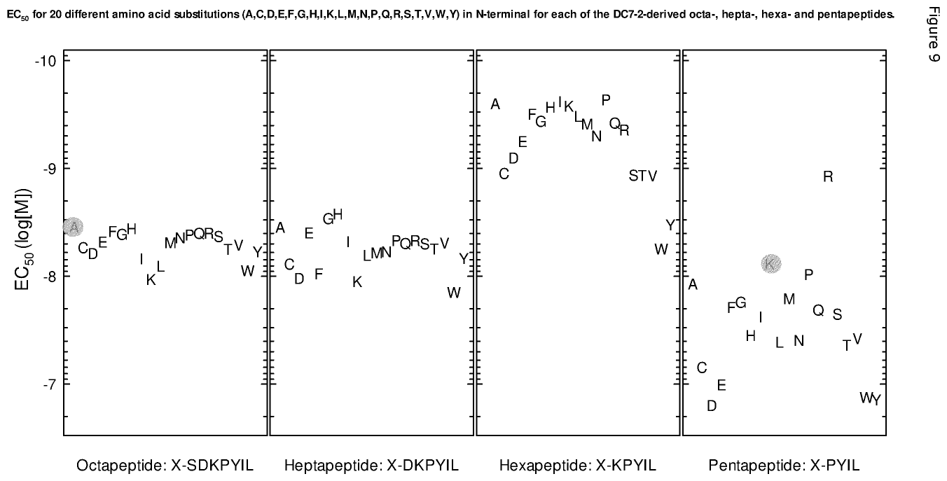

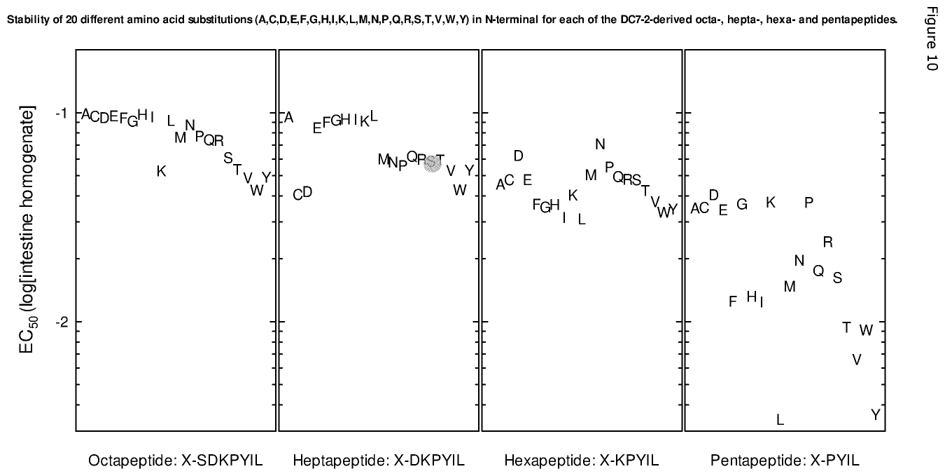

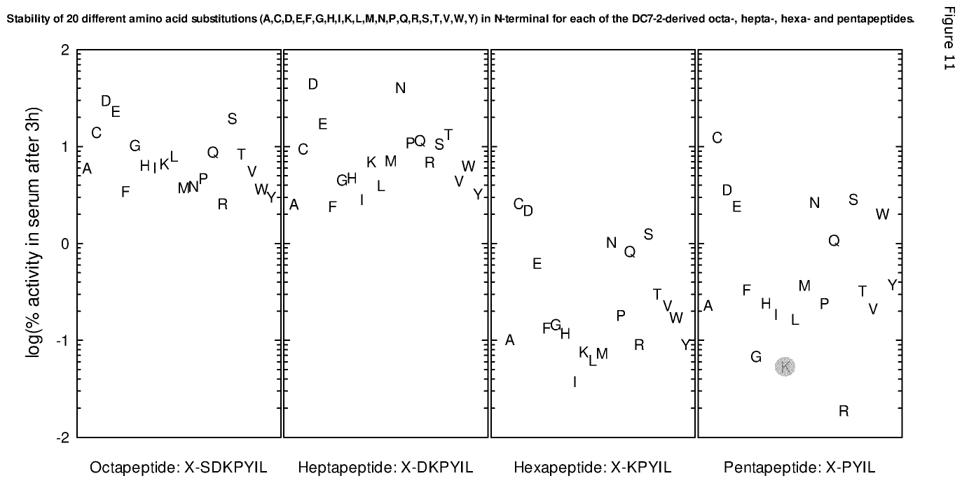

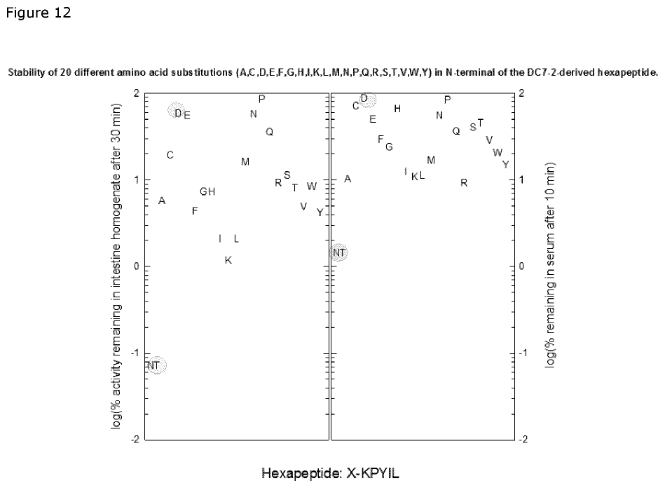

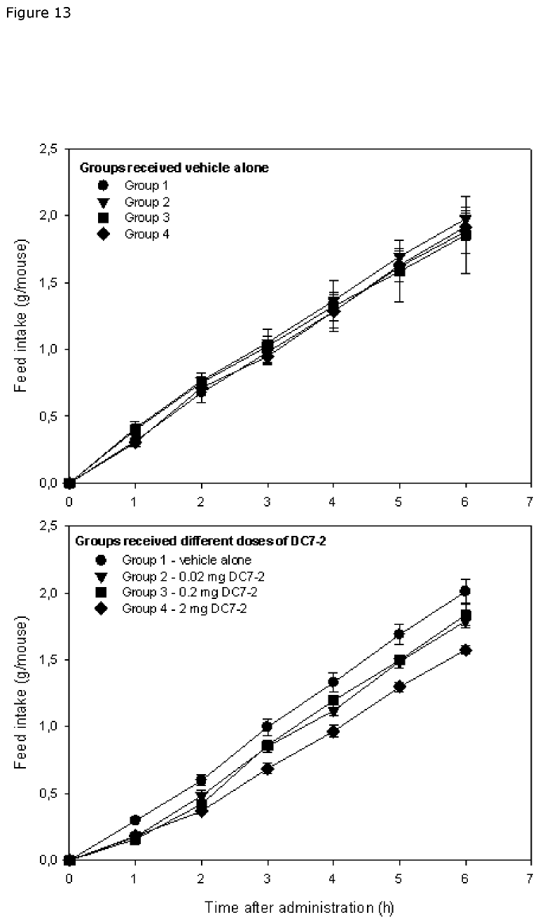

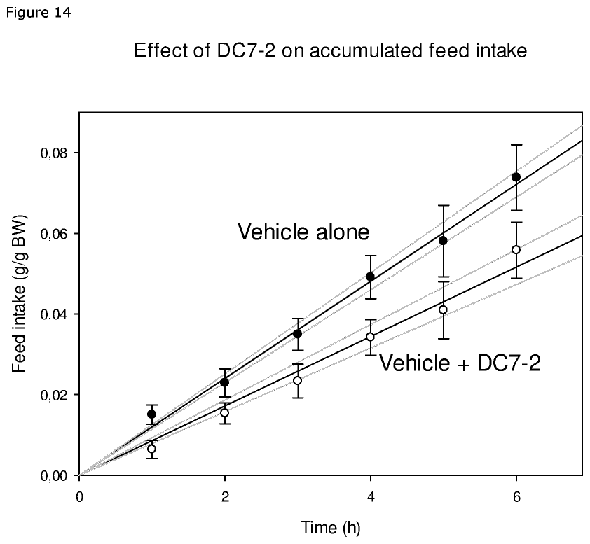

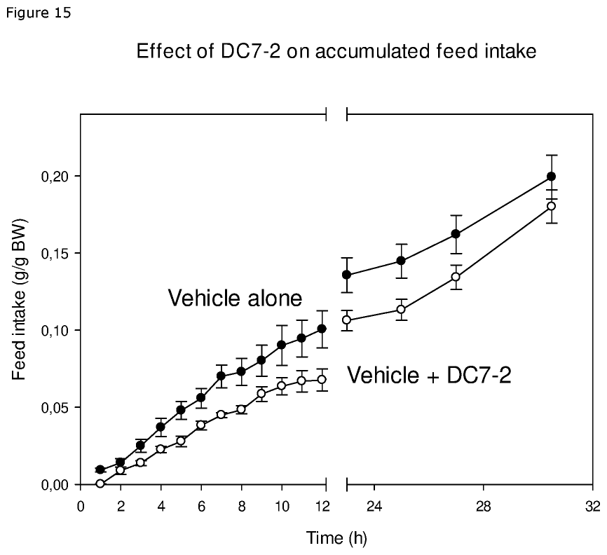

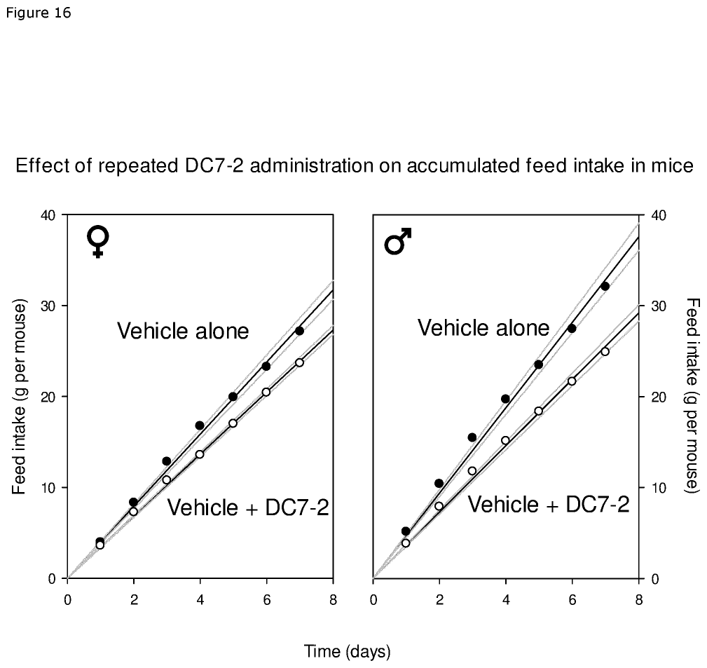

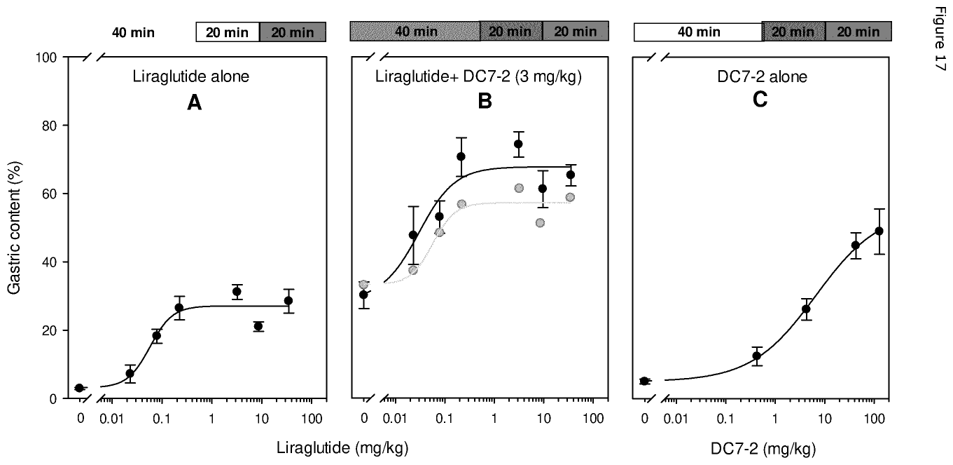

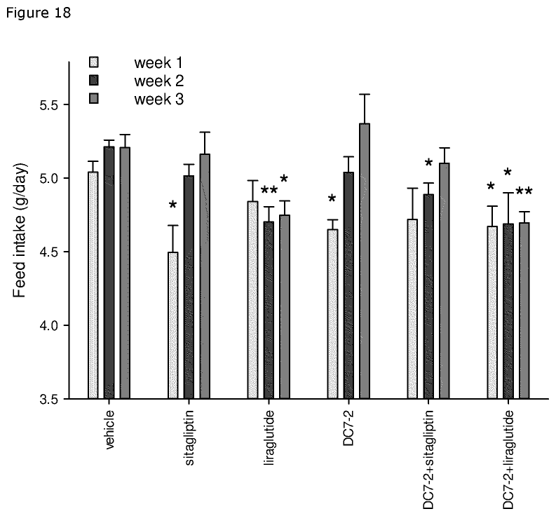

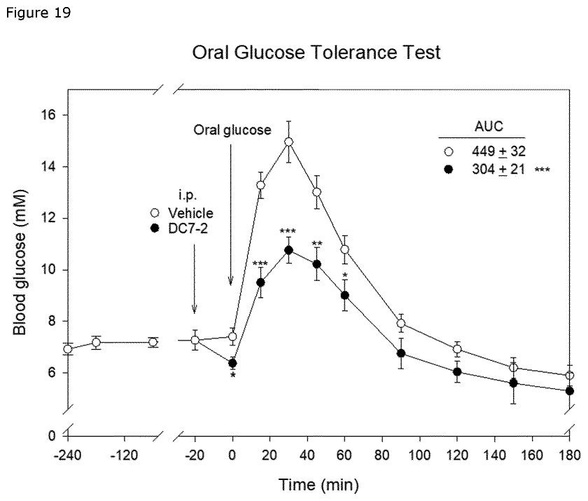

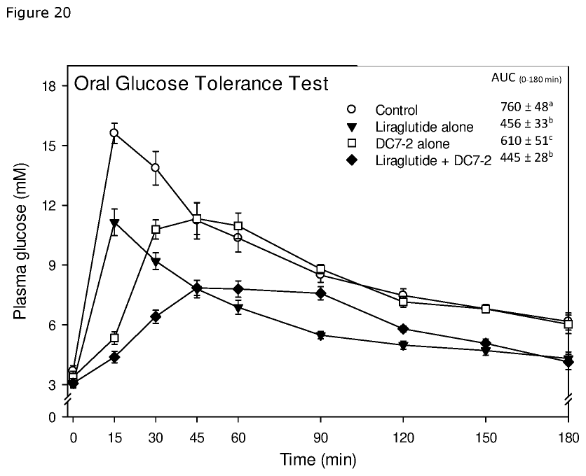

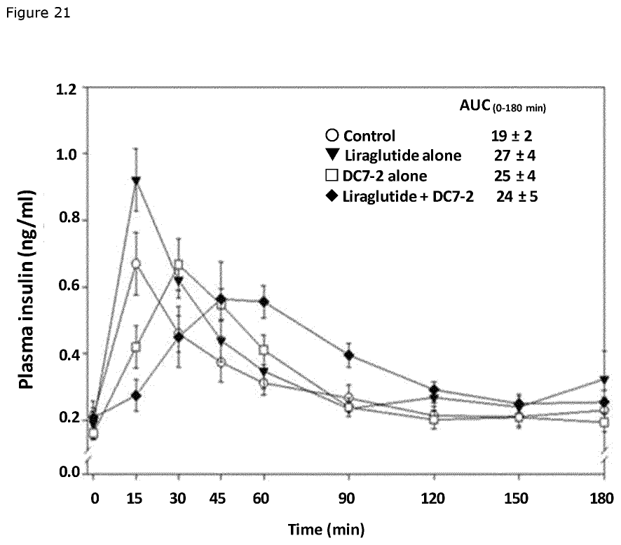

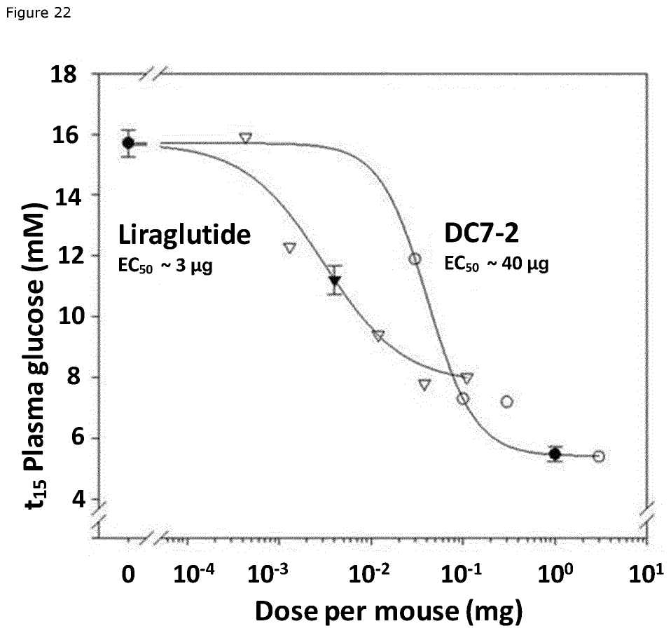

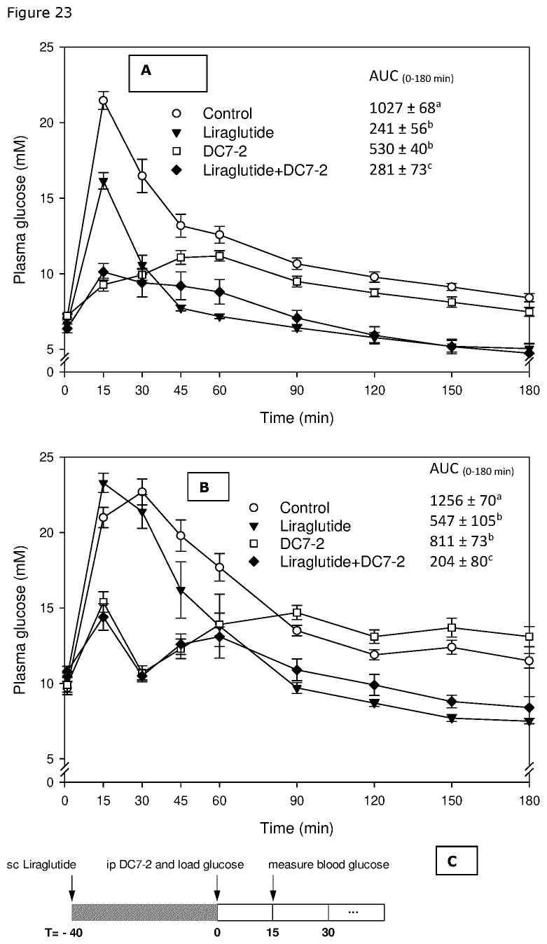

Truncation from the amino-terminal or from the carboxy-terminal end of ASDKPYIL has different consequences. Deleting the carboxy-terminal leucine reduces potency more than two orders of magnitude in mIC cells and abrogates activity in hIC. Peptides with further deletions of 2, 3 or 4 amino acids from the carboxy-terminus are without activity. Deleting the first three amino acids from the amino-terminus has no big impact on activity. However, the fourth amino acid, lysine, is critical, since PYIL has two orders of magnitude lower activity compared with the full sequence in mIC and no activity in hIC.

FIG. 3. Identification of critical residues in ASDKPYIL (d-Ala (AD) scan). Systematic replacement of all residues in ASDKPYIL with the d-isomer of alanine and corresponding biological activity. Results show that 1) the last four amino acids (PYIL, SEQ ID NO:4) are critical, 2) replacing K reduces potency more than 30-fold, 3) replacing the aspartic residue improves potency almost 10-fold, and 4) alanine and serine on the first two positions are without importance.

FIG. 4. Stability of peptides in rodent intestine.

0.001 mg/ml of the indicated peptides were incubated with pieces of rodent intestine (mouse and rat intestine gave similar results) for up to 10 minutes at 37° C. Recovery of activity was tested with dose-response curves as indicated.

FIG. 5. Stability of peptides in rodent intestine. EC₅₀ values for different peptides and different incubation times were calculated from FIG. 8 and recovered activity plotted as a function of time.

FIG. 6. Comparison of the sequences of three known gut hormones, neurotensin, neuromedin N and xenin with that of DC7-2 (ASDKPYIL). The PYIL sequence is conserved, although Y is replaced by W in xenin.

FIG. 7. Comparison of the DC7-2 sequence (aa 891-898) in isoforms of a-actinin 2 (Hs: Homo sapiens ACTN1-4) and conservation between species (Dm: Drosophila melanogaster; Ce: Caenorhabditis elegans; Dd: Dictyostelium discoideum; Sp: Schizosaccharomyces pombe; Dr: Danio rerio)

FIG. 8. 24 Balb/c female mice, 10-11 weeks, 20-22 g, were acclimatized to 12 h dark light cycle and placed single-housed in metabolic cages. Following administration of the indicated doses of DC7-2, feed and water intake was monitored for 6 h.

FIG. 9. Summary of cell signaling activities of N-terminal substitutions in octa-, hepta-, hexa- and pentapeptides based on the sequence of DC7-2. Single-letter abbreviations for the 20 amino acids are shown on the plot centered at the corresponding EC50. The native amino acid in DC7-2 is marked with a grey circle for each of the peptides.

FIG. 10. Stability of DC7-2 families of peptides in intestine homogenates. Single-letter abbreviations for the 20 amino acids are shown on the plot with the corresponding stability expressed as the logarithm to the concentration of intestine homogenate that degrades half of the activity of peptide. All peptides were incubated at 10−5 M with various dilutions of a homogenate of the entire small intestine (pool from 20 mice). After incubation for 90 min at 37° C., degradation was stopped by addition of 1 M phosphoric acid (final 0.4 M, pH ˜1.2). Each peptide incubation mix was neutralized with NaOH and immediately tested for activity in intestinal cells. Control for zero degradation, i.e. addition of phosphoric acid before addition of intestine homogenate, was included for each peptide. The native amino acid in DC7-2 is marked with a grey circle for each of the peptides.

FIG. 11. Stability of DC7-2 families of peptides in serum.

FIG. 12. Stability of X-KPYIL hexapeptides in intestine homogenate and serum.

FIG. 13. 24 Balb/c female mice, 10-11 weeks, 20-22 g, were acclimatized to 12 h dark light cycle. Mice were divided into four groups each of six mice and placed single-housed in metabolic cages. Mice were then administered vehicle alone (day 1) for monitoring of feed and water intake for 6 h. On day 3, the same groups received the indicated doses of DC7-2, and feed and water intake was monitored for 6 h.

FIG. 14. Swiss Webster male mice, 25-30 g, were acclimatized to 12 h dark/light cycle and placed single-housed in cages. Following administration just prior to onset of dark cycle of vehicle alone (0.5 ml of PBS w 1% of BSA) or vehicle+DC7-2, feed intake was monitored every hour for 6 h (during dark cycle). Mean and SEM from four experiments, each with 6-8 mice per treatment. Data were fitted with linear regression (R2>0.99) and 95% confidence intervals are shown as grey lines. Accumulated feed intake for treatment with DC7-2 was 64%+/−5% compared with control for these four experiments.

FIG. 15. Swiss Webster male mice, 25-30 g, were acclimatized to 12 h dark/light cycle and placed single-housed in cages. Following administration just prior to onset of dark cycle of vehicle alone (0.5 ml of PBS w 1% of BSA) or vehicle+DC7-2, feed intake was monitored every hour for 12 h (during dark cycle) and then intermittently up to 30 h.

FIG. 16. Swiss Webster male (25-30 g) or female (20-25 g) mice were acclimatized to 12 h dark/light cycle and placed in groups of 6-8 mice per cage. Vehicle (0.5 ml of PBS w 1% of BSA) alone or vehicle+DC7-2 was administered three times per day (08:00; 16:00; 24:00), and feed intake was monitored daily for a week. Data were fitted with linear regression (R2>0.99) and 95% confidence intervals (grey lines).

FIG. 17. The octapeptide (ASDKPYIL, SEQ ID NO:6) and the GLP-1 analogue, liraglutide, had a synergistic effect on delaying gastric emptying. (A) Liraglutide dose-dependently delayed gastric emptying by subcutaneous administration (250 μl/mouse at t=−40 min, n=6 for 0.02 mg, 8.7 mg and 35 mg of liraglutide/mouse, n=14 for 0 mg/mouse, n=16 for 0.08 mg of liraglutide/mouse and n=19-22 for the all other doses), (B) Liraglutide and DC7-2 have a syngeneic effect on gastric emptying (250 μl, different concentrations of liraglutide s.c. at t=−40 min and 3 mg of DC7-2/mouse at t=−20 min, n=25 for 0 mg of liraglutide+3 mg DC7-2/mouse, n=6-7 for 0.02 mg, 8.7 mg and 35 mg of liraglutide+3 mg of DC7-2/mouse and n=14-18 for the others), (C) DC7-2 dose-dependently delayed gastric emptying by intraperitoneal administration (250 μl/mouse at t=−20 min, n=3 for 3 mg of DC7-2/mouse, n=11 for 1 mg of DC7-2/mouse, n=6-8 for other doses of DC7-2).

FIG. 18. DC7-2, liraglutide and sitagliptin reduces feed intake after repeated daily administration. Chronic effects on accumulative feed intake (g/day) after subcutaneous administration of liraglutide (3 μg/mouse) and intraperitoneal injection of DC7-2 (1 mg/mouse) and the DPP IV inhibitor, sitagliptin (0.025 mg/mouse). Mice had free access to water and feed in this 3-week study (n=14-18 per group). Significance (P<0.05) is shown as * compared to the corresponding vehicle control.

FIG. 19. Mice were randomly assigned into four groups each with four mice and fasted for 4 h and were injected s.c. with vehicle or vehicle containing 1 mg of DC7-2 by i.p. administration 20 min prior to oral glucose challenge (300 ul of 20% of glucose in 40% of PEG in water). Blood was sampled from a tail vein and blood glucose was determined with strips and reader (Bayer). Results are mean+/−SEM (n=28 per group) and are combined from seven separate experiments. Area under the curve (AUC) was calculated for each group. *P<0.05; **P<0.01; ***P<0.001.

FIG. 20. Mice were randomly assigned into four groups each with four mice after o/n fasting and were injected s.c. with vehicle or Liraglutide 40 min prior to glucose challenge, then immediately before glucose challenge mice received either vehicle alone (PBS/1% BSA) or vehicle containing 1 mg of DC7-2 by i.p. administration, followed by an oral glucose load (1% of bodyweight of 20% of glucose in 40% of PEG). Blood was sampled into EDTA capillaries and centrifuged to obtain plasma that was subsequently pooled for the four mice in each experiment. Plasma glucose was determined with strips and reader (Bayer). Results are mean+/−SEM (n=28 per group) and are combined from seven separate experiments. Area under the curve (AUC) was calculated for each group and values with different superscripts are significantly different (P<0.001).

FIG. 21. Insulin levels in plasma determined in the same samples as used for plasma glucose in FIG. 20.

FIG. 22. Dose-response for Liraglutide and DC7-2 on plasma glucose levels 15 min after oral glucose load. Filled symbols are mean and SEM from nine experiments (each with four mice per group), open symbols are from one experiment with four mice per group.

FIG. 23. Lean-control (A) and DIO mice (C57Bl/6J mice inbred) (B) were randomly assigned into four groups after 5-hour fasting and treated with OGTT. Study represented in (C). Results are mean+/−SEM (n=8 mice per group), Area under the curve (AUC) with different superscripts are significantly different (P<0.01).

### DETAILED DISCLOSURE OF THE INVENTION

The inventors of the present invention have found novel polypeptides that may be used to induce signalling in intestinal cells and may consequently induce satiety. Although a specific peptide has been identified from a proteolytic digest of muscle-specific alpha-actinin-2 protein, it is envisioned that similar polypeptides will bind the same receptors in the intestine and provide the same biological activity, i.e. signal to induce satiation and satiety. Similar peptides may contain e.g. conservative substitutions or be truncated. The rationale for using the polypeptides of the invention is that the energy content due to the relatively small length of the peptide is low as compared to the effect on satiety.

### Definitions

When terms such as “one”, “a” or “an” are used in this disclosure they mean “at least one”, or “one or more” unless otherwise indicated. Further, the term “comprising” is intended to mean “including” and thus allows for the presence of other constituents, features, conditions, or steps than those explicitly recited.

In some specific embodiments, the first 1, 2, or 3 amino acids in the N-terminal of the amino acid sequences according to the invention are in the D-form. It is assumed that the N-terminal trimming and thereby degradation of the peptides are somewhat delayed by having amino acids of the D-form in the N-terminal of these polypeptides. Alternatively and in some embodiments, the first 1, 2, or 3 amino acids in the N-terminal of the amino acid sequences according to the invention are amino acids in beta or gamma forms. Beta amino acids have their amino group bonded to the beta carbon rather than the alpha carbon as in the 20 standard natural amino acids. A capital D-letter subscript after the letter representing the amino acid residue designate herein amino acids specified to be in D-form, such as WD referring to a tryptophan in D-form. A capital L-letter subscript after the letter representing the amino acid residue designate herein amino acids specified to be in L-form, such as WL referring to a tryptophan in L-form. If not otherwise indicated, an amino acid is in its natural L-form.

Alternatively, the first 1, 2, or 3 amino acids in the N-terminal of the amino acid sequences according to the invention may be modified by incorporation of protective groups, e.g. fluorine, or alternatively cyclic amino acids or other suitable non-natural amino acids are used.

A “variant” or “analogue” of a peptide refers to a peptide having an amino acid sequence that is substantially identical to a reference peptide, typically a native or “parent” polypeptide, or a polypeptide of formula I or II. The peptide variant may possess one or more amino acid substitutions, deletions, and/or insertions at certain positions within the native amino acid sequence. The “variant” within this definition still has functional activity. In some embodiment a variant has at least 80% sequence identity with the reference polypeptide. In some embodiments a variant has at least 85% sequence identity with the reference polypeptide. In other embodiments a variant has at least 90% sequence identity with the reference polypeptide. In a further embodiment a variant has at least 95% sequence identity with the reference polypeptide.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). A particular form of conservative amino acid substitutions include those with amino acids, which are not among the normal 20 amino acids encoded by the genetic code. Since preferred embodiments of the present invention entail use of synthetic peptides, it is unproblematic to provide such “non-naturally occurring” amino acid residues in the peptides disclosed herein, and thereby it is possible to exchange the natural saturated carbon chains in the side chains of amino acid residues with shorter or longer saturated carbon chains—for instance, lysine may be substituted with an amino acid having a side chain —(CH2)nNH3, where n is different from 4, and arginine may be substituted with an amino acid having the side chain (CH2)nNHC(═NH2)NH2, where n is different from 3, etc. Similarly, the acidic amino acids aspartic acid and glutamic acid may be substituted with amino acid residues having the side chains —(CH₂)nCOOH, where n>2.

The polypeptides of this invention may in some embodiments benefit from having higher stability than polypeptides containing only naturally occurring amino acids, and its modification enables to have much higher stability, such as a modification in the N-terminal of the polypeptide.

Accordingly and in some embodiments, the polypeptides of this invention have at their N-terminal a protection group, such as a protection group selected from the group consisting of acetyl group, fluorenyl methoxy carbonyl group, formyl group, palmitoyl group, myristyl group, stearyl group and polyethylene glycol (PEG).

The active peptide may also be di- or multimerized, e.g. through cross-linking with suitable di- or multivalent chemical cross-linkers, e.g. disuccinimidyl suberate, containing spacers of different length, e.g. 10-100 Å, and different functionality, e.g. homo- or heterofunctional, for coupling through non-critical amino or other reactive groups. Alternatively, photoactivation or enzymatic cross-linking may be used to increase stability and potency in vivo.

The modifications of peptides described above greatly increase the stability of the peptides of this invention. The term used herein “stability” refers to in vivo stability, such as the stability in the gut of a subject receiving such polypeptide. The protection group described above protects the peptides from the attack of protease in vivo.

The polypeptides according to the invention may be derived from a proteolytic digests of meat and be resistant to pepsin degradation. Accordingly, in some embodiments a polypeptide according to the invention may only contain naturally occurring amino acids.

In other embodiments, a polypeptide according to the invention is more stable towards degradation in the gastrointestinal tract, e.g. as measured in a stability assay described in the examples of the present invention, as compared to a control peptide. In some embodiments, a polypeptide according to the invention is more stable towards degradation in the gastrointestinal tract, e.g. measured in a stability assay described in the examples of the present invention as compared to a control peptide with the sequence RRPYIL, (SEQ ID NO:39).

In some embodiments, a polypeptide according to the invention has an half-life (T½) of degradation in vivo in the gut or in vitro, e.g. measured in a stability assay described in the example 2 of the present invention, which is higher than 2 min, such as higher than 4 min, such as higher than 6 min, such as higher than 8 min, such as higher than 10 min, such as higher than 15 min, such as higher than 20 min, such as higher than 25 min, such as higher than 30 min, such as higher than 35 min, such as higher than 40 min, such as higher than 45 min, such as higher than 50 min, such as higher than 55 min, such as higher than 60 min.

The term “substantially identical” in the context of two amino acid sequences means that the sequences, when optimally aligned, such as by the programs GAP or BESTFIT using default gap weights, share at least about 50, at least about 60, at least about 70, at least about 80, at least about 90, at least about 95, at least about 98, or at least about 99 percent sequence identity. In some embodiments, when measuring the sequence identity between two different peptide sequences, a gap of one or two amino acids is allowed when the two peptide sequences are aligned without having any influence on the value of sequence identity. In some embodiments, a residue position that is not identical differ by only a conservative amino acid substitution. Sequence identity is typically measured using sequence analysis software. Protein analysis software matches similar sequences using measures of similarity assigned to various substitutions, deletions and other modifications, including conservative amino acid substitutions. For instance, the publicly available GCG software contains programs such as “Gap” and “BestFit” which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences can also be compared using FASTA or ClustalW, applying default or recommended parameters. A program in GCG Version 6.1., FASTA (e.g., FASTA2 and FASTA3) provides alignments and percent sequence identity of the regions of the best overlap between the query and search sequences (Pearson, Methods Enzymol. 1990; 183:63-98; Pearson, Methods Mol. Biol.

2000; 132:185-219). Another preferred algorithm when comparing a sequence to a database containing a large number of sequences from various organisms is the computer program BLAST, especially blastp, using default parameters. See, e.g., Altschul et al., J. Mol. Biol. 1990; 215:403-410; Altschul et al., Nucleic Acids Res. 1997; 25:3389-402 (1997); each herein incorporated by reference. “Corresponding” amino acid positions in two substantially identical amino acid sequences are those aligned by any of the protein analysis software mentioned herein, typically using default parameters.

The term “functional activity” as used herein refers to a polypeptide that stimulates cell signalling measured as fluorescence by elevated intracellular calcium or cellular release of gut hormones, such as measured in the signalling assays described in the examples. The functional activity of a variant may exhibit at least about 25%, such as at least about 50%, such as at least about 75%, such as at least about 90% of the specific activity of a reference polypeptide, such as the octapeptide ASDKPYIL, when tested in the assays as described herein. Alternatively, the functional activity of a variant may exhibit higher activity than a reference polypeptide, such as the octapeptide ASDKPYIL, when tested in the assays as described herein.

An “isolated” molecule is a molecule that is the predominant species in the composition wherein it is found with respect to the class of molecules to which it belongs (i.e., it makes up at least about 5% of the type of molecule in the composition and typically will make up at least about 10%, at least about 20%, at least about 30%, at least about 40%, at least about 50%, at least about 60%, at least about 70%, at least about 80%, at least about 85%, at least about 90%, at least about 95%, or more of the species of molecules, e.g., peptides, in the composition). Commonly, a composition of a specific peptide sequence may exhibit 90%-99% homogeneity for peptides in the context of all present peptide species in the composition or at least with respect to substantially active peptide species in the context of proposed use. If produced synthetically, a composition of a specific peptide sequence will exhibit 98%-99%, or even higher and close to 100% homogeneity for peptides in the context of all present peptide species in the composition or at least with respect to substantially active peptide species in the context of proposed use.

Unless otherwise indicated the polypeptides within the present invention is a linear sequence of amino acids. The term “linear sequence” as used herein refers to the specific sequence of amino acids connected by standard peptide bonds in standard N- to C-terminal direction. The peptide may contain only peptide bonds. In some embodiments however, a second part of a peptide sequence may be bound to and continue from the side chain of a terminal amino acid in a first part of an amino acid sequence. Also the term does not exclude that an amino acid within a sequence, such as within AA1-AA8, may be connected, such as through the side chains, with another amino acid at a distant location within the peptide sequence, such as a distant location within AA1-AA8.

GLP-1 Receptor Agonists

A “GLP-1 receptor agonist” as used herein refers to a peptide hormone that binds and are agonists of the GLP-1 receptor. A GLP-1 receptor agonist may be or may be derived from the native biologically active form of GLP-1, i.e. GLP-1(7-37). In some embodiments, a GLP-1 receptor agonist may be a GLP-1 peptide comprising no more than 5, such as no more than 4 or no more than 3, amino acid residues which have been substituted, inserted or deleted as compared to GLP-1(7-37). The GLP-1 receptor agonist may be a GLP-1 peptide, a GLP-1 fragment, derivative or analogue. The GLP-1 receptor agonist may be suitable for once daily administration or the GLP-1 receptor agonist may be suitable for once weekly administration.

The GLP-1 receptor agonist may be selected from the group consisting of: GLP-1(7-37); GLP-1(7-36) amide; Exenatide; Exenatide LAR; Liraglutide; Semaglutide; Taspoglutide; Albiglutide; Lixisenatide; Dulaglutide; and Oxyntomodulin. The GLP-1 receptor agonist may be Liraglutide or Semaglutide. The GLP-1 receptor agonist may be a long-acting GLP-1 derivative or analogue. The long-acting GLP-1 derivative or analogue may be selected from the group consisting of: Liraglutide; Semaglutide; Taspoglutide; Albiglutide; Taspoglutide; Dulaglutide; and Exenatide LAR.

A GLP-1 receptor agonist may be a GLP-1 fragment, derivative or analogue, e.g. a GLP-1 analogue or GLP-1 derivative.

A “GLP-1 peptide” as used herein refers to the human Glucagon-Like Peptide-1 (GLP-1(7-37)), with the sequence HAEGTFTSDVSSYLEGQAAKEFIAWLVKGRG (SEQ ID NO:1006), or an analogue thereof. The peptide having the sequence of SEQ ID NO: 1006 may be designated human GLP-1, or “native” GLP-1, or native GLP-1(7-37). A GLP-1 receptor agonist may be as described in e.g. WO 9808871, WO 9943706, U.S. Pat. No. 5,424,286, WO 0009666, US patent applications US2016136246A1 or 2009042781A1.

In the context of the present invention, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

The terms “patient” and “subject” refer to any human or animal that may be treated using the methods of the present invention.

Many aspects of the present invention relates to the use of polypeptides or compositions to promote satiety in a subject. The underlying cause of a metabolic syndrome or disorder that may treated by the polypeptides or compositions according to the invention, is an overconsumption of calories, while still not feeling satiety. By inducing or promoting satiety or reducing feed intake in a subject, such total amounts of calories, including calories derived from fat and carbohydrates are reduced in the subject. Accordingly, the polypeptides and compositions of the invention may be used in preventing or reducing a metabolic syndrome or disorder, such as obesity, insulin-deficiency or insulin-resistance related disorders, Diabetes Mellitus (such as, for example, Type 2 Diabetes), glucose intolerance, abnormal lipid metabolism, atherosclerosis, hypertension, cardiac pathology, stroke, non-alcoholic fatty liver disease, hyperglycemia, hepatic steatosis, dyslipidemia, dysfunction of the immune system associated with overweight and obesity, cardiovascular diseases, high cholesterol, elevated triglycerides, asthma, sleep apnoea, osteoarthritis, neuro-degeneration, gallbladder disease, syndrome X, inflammatory and immune disorders, atherogenic dyslipidemia and cancer.

Preparation of Polypeptides of the Invention

The invention also relates to a method of preparing polypeptides of the invention as mentioned above. The method of synthesis or preparation thereof includes, but is not limited to recombinant (whether produced from cDNA, genomic DNA, synthetic DNA or other form of nucleic acid), synthetic, and transgenic means.

The polypeptides of the invention described herein may be produced by means of recombinant nucleic acid techniques. In general, a nucleic acid sequence encoding the desired polypeptide is then inserted into an expression vector, which is in turn transformed or transfected into host cells.

As an alternative and also the preferred option, the polypeptides of the invention are produced by synthetic means, i.e. by polypeptide synthesis. In some embodiments, the invention relates to a method of manufacturing an analogue comprising non-natural amino acids from about 5 total residues to about 20 total residues. In some embodiments, an analogue comprises 1, 2, 3, 4, 5, 6, 7, 8, 9, 10, 11, 12, 13, 14, 15, 16, 17, 18, 19, or 20 non-natural amino acids, such as any one of the following non-naturally occurring amino acid residues.

The polypeptides of the present invention can also comprise non-naturally occurring amino acid residues. Non-naturally occurring amino acids include, without limitation, beta-alanine, desaminohistidine, trans-3-methylproline, 2,4-methanoproline, cis-4-hydroxyproline, trans-4-hydroxyproline, N-methylglycine, allo-threonine, methylthreonine, hydroxyethylcys-teine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, nor-valine, 2-azaphenylalanine, 3-azaphenylalanine, 4-azaphenylalanine, and 4-fluorophenylalanine. Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Polypeptides are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oo-cytes by microinjection of mutated mRNA and chemically aminoacylated suppressor tRNAs (Turcatti et al., J. Biol. Chem. 271:19991-8, 1996). Within a third method, E. coli cells are cul-tured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

As another alternative to synthetic preparation, the polypeptides of the invention may be purified from any natural source containing such polypeptide, such as from the proteolytic hydrolysate of muscle tissue, such as any source containing alpha-actinin-2 protein, such as by the methods described in the example section.

Accordingly, in some embodiments the sequence of the polypeptides of the invention is derived from a sequence found in nature, such as a fragment of alpha-actinin-2 protein.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). They may be purified by affinity chromatography on an antibody column. Additional purification may be achieved by conventional chemical purification means, such as high performance liquid chromatography. Other methods of purification, including barium citrate precipitation, are known in the art, and may be applied to the purification—see, for example, Scopes, R., Protein Purification, Springer-Verlag, N.Y., 1982.

For the methods of the invention including the therapeutic purposes it is not critical to have a high purity of a specific peptide of the invention. However, the higher the concentration of a specific peptide of the invention the higher is the effect in terms of inducing satiation and satiety relative to amount of total protein and total amount of calories consumed by the subject receiving the composition of polypeptides. It is to be understood that the idea of the invention is to administer polypeptides that induce satiation or satiety without administering a lot of calories to the subject.

In some embodiments the compositions of polypeptides of the invention are substantially pure. Thus, in an embodiment of the invention the polypeptides of the invention are purified to at least about 90 to 95% homogeneity, preferably to at least about 98% homogeneity. Purity may be assessed by e.g. HPLC and amino-terminal amino acid sequencing.

Administration and Pharmaceutical Compositions

Administration of the polypeptides according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Some kind of oral administration is preferred since these types of polypeptides are derived from a source that naturally has to pass through the mouth and to the intestinal mucosa.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

One of skill in the art will recognize that the appropriate dosage of the compositions and pharmaceutical compositions may vary depending on the individual being treated and the purpose. For example, the age, body weight, and medical history of the individual patient may affect the therapeutic efficacy of the therapy. Further, a lower dosage of the composition may be needed to produce a transient cessation of symptoms, while a larger dose may be needed to produce a complete cessation of symptoms associated with the disease, disorder, or indication. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages may also depend on the strength of the particular polypeptide of the invention chosen for the pharmaceutical composition.

The dose of the composition or pharmaceutical compositions may vary. The dose of the composition may be once per day. In some embodiments, multiple doses may be administered to the subject per day. In some embodiments, the total dosage is administered in at least two application periods, In some embodiments, the period can be an hour, a day, a month, a year, a week, or a two-week period. In an additional embodiment of the invention, the total dosage is administered in two or more separate application periods, or separate doses.

In some embodiments, subjects can be administered the composition in which the composition is provided in a daily dose range of about 0.0001 mg/kg to about 5000 mg/kg of the weight of the subject. The dose administered to the subject can also be measured in terms of total amount of polypeptide of the invention administered per day. In some embodiments, a subject is administered from about 0.001 to about 3000 milligrams of polypeptide of the invention per day. In some embodiments a subject is administered up to about 2000 milligrams of polypeptide of the invention per day. In some embodiments, a subject is administered up to about 1800 milligrams of polypeptide of the invention per day. In some embodiments, a subject is administered up to about 1600 milligrams of polypeptide of the invention per day. In some embodiments, a subject is administered up to about 1400 milligrams of polypeptide of the invention per day. In some embodiments, a subject is administered up to about 1200 milligrams of polypeptide of the invention per day. In some embodiments, a subject is administered up to about 1000 milligrams of polypeptide of the invention per day. In some embodiments, a subject is administered up to about 800 milligrams of polypeptide of the invention per day. In some embodiments, a subject is administered from about 0.001 milligrams to about 700 milligrams of polypeptide of the invention per dose. In some embodiments, a subject is administered up to about 700 milligrams of polypeptide of the invention per dose. In some embodiments, a subject is administered up to about 600 milligrams of polypeptide of the invention per dose. In some embodiments, a subject is administered up to about 500 milligrams of polypeptide of the invention per dose. In some embodiments, a subject is administered up to about 400 milligrams of polypeptide of the invention per dose. In some embodiments, a subject is administered up to about 300 milligrams of polypeptide of the invention per dose. In some embodiments, a subject is administered up to about 200 milligrams of polypeptide of the invention per dose. In some embodiments, a subject is administered up to about 100 milligrams of polypeptide of the invention per dose. In some embodiments, a subject is administered up to about 50 milligrams of polypeptide of the invention per dose.

A composition, wherein a polypeptide of the invention is added may be any food composition, food product, or food ingredient. Here, the term “food” is used in a broad sense—and covers food for humans as well as food for animals (i.e. a feed). In a preferred aspect, the food is for human consumption. The food may be in the form of a solution or as a solid—depending on the use and/or the mode of application and/or the mode of administration.

When used as—or in the preparation of—a food—such as functional food—the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

The composition of the present invention may be used as a food ingredient.

As used herein the term “food ingredient” includes a formulation which is or can be added to functional foods or foodstuffs as a nutritional supplement. The term food ingredient as used here also refers to formulations which can be used at low levels in a wide variety of products that require gelling, texturising, stabilising, suspending, film-forming and structuring, retention of juiciness and improved mouthfeel, without adding viscosity.

The food ingredient may be in the form of a solution or as a solid—depending on the use and/or the mode of application and/or the mode of administration.

The composition of the present invention may be—or may be added to—food supplements.

The composition of the present invention may be—or may be added to—functional foods.

As used herein, the term “functional food” means food which is capable of providing not only a nutritional effect and/or a taste satisfaction, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Surveys have suggested that consumers place the most emphasis on functional food claims relating to heart disease. Preventing cancer is another aspect of nutrition which interests consumers a great deal, but interestingly this is the area that consumers feel they can exert least control over. In fact, according to the World Health Organization, at least 35% of cancer cases are diet-related. Furthermore claims relating to osteoporosis, gut health and obesity effects are also key factors that are likely to incite functional food purchase and drive market development.

The composition of the present invention can be used in the preparation of or added to food products such as one or more of: jams, marmalades, jellies, dairy products (such as milk or cheese), meat products, poultry products, fish products, vegetable-based soups, and bakery products.

By way of example, the composition of the present invention can be used as ingredients to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, breakfast cereals, instant noodles and cup noodles, instant soups and cup soups, balanced foods and drinks, sweeteners, texture improved snack bars, fibre bars, bake stable fruit fillings, care glaze, chocolate bakery filling, cheese cake flavoured filling, fruit flavoured cake filling, cake and doughnut icing, heat stable bakery filling, instant bakery filling creams, filing for cookies, ready-to-use bakery filling, reduced calorie filling, adult nutritional beverage, acidified soy/juice beverage, aseptic/retorted chocolate drink, bar mixes, beverage powders, calcium fortified soy/plaim and chocolate milk, calcium fortified coffee beverage.

A composition according to the present invention can further be used as an ingredient in food products such as American cheese sauce, anti-caking agent for grated & shredded cheese, chip dip, cream cheese, dry blended whip topping fat free sour cream, freeze/thaw dairy whipping cream, freeze/thaw stable whipped topping, low fat & lite natural cheddar cheese, low fat Swiss style yoghurt, aerated frozen desserts, and novelty bars, hard pack ice cream, label friendly, improved economics & indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

For certain aspects, preferably the foodstuff is a beverage.

For certain aspects, preferably the foodstuff is a bakery product—such as bread, Danish pastry, biscuits or cookies.

The present invention also provides a method of preparing a food or a food ingredient, the method comprising mixing a polypeptide according to the present invention or the composition according to the present invention with another food ingredient.

### SPECIFIC EMBODIMENTS OF THE INVENTION

One aspect of the invention related to a combination of

1) a first polypeptide comprising the amino acid sequence
AA1-AA2-AA3-K-AA5-AA6-AA7-AA8 (formula I; SEQ ID NO:1),

wherein AA1 when present is an amino acid selected from A, L, I, and V; AA2 when present is an amino acid selected from S, T, G, A, N, E and D; AA3 when present is an amino acid selected from D, E, and G; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and

2) a second peptide hormone, such as a polypeptide being a GLP-1 receptor agonist.

Alternatively the invention related to a combination according to any one of the previous claims, wherein said first polypeptide is a polypeptide consisting of the amino acid sequence

R1-AA1-AA2-AA3-K-AA5-AA6-AA7-AA8-R2 (formula II, SEQ ID NO:2),

wherein AA1 when present is an amino acid selected from A, L, I, and V; AA2 when present is an amino acid selected from S, T, G, A, N, E and D; AA3 when present is an amino acid selected from D, E, and G; AA5 is selected from P, N, S, D, A, T, K, and G; AA6 is selected from Y, N, I, W, and F; AA7 is selected from I, L, R, and V; AA8 is selected from L, I, V, S, M, and T; R1 defines the N-term (—NH2) or a protection group; R2 defines the C-term (—COOH).

Another aspect of the invention related to a method of promoting satiety or for reducing feed intake in a subject, comprising enteral administering to a subject in need thereof a combination of

1) a first polypeptide comprising or consisting of the amino acid sequence
AA1-AA2-AA3-AA4-AA5-AA6-AA7-AA8 (formula III, SEQ ID NO:3),

wherein AA1 when present is an amino acid selected from A, L, I, and V; AA2 when present is an amino acid selected from S, T, G, A, N, E and D; AA3 when present is an amino acid selected from D, R, K, E, and G; AA4 is an amino acid selected from K and R; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and

2) a second peptide hormone, such as a polypeptide being a GLP-1 receptor agonist.

In the following AA1-AA8 may refer to the amino acids of formula I, II, or III.

In some embodiments said first polypeptide comprises or consists of AA1-AA2-AA3-K—P-Y-I-L. In some embodiments said first polypeptide comprises or consists of AA1-AA2-AA3-AA4-P—Y-I-L. In some embodiments AA1 is absent. In some embodiments AA1 is any one natural amino acid selected from Y, W, V, T, S, R, Q, P, N, M, L, K, I, H, G, F, E, D, C, and A. In some embodiments AA2 is absent. In some embodiments AA2 is any one natural amino acid selected from Y, W, V, T, S, R, Q, P, N, M, L, K, I, H, G, F, E, D, C, and A. In some embodiments AA3 is absent. In some embodiments AA1 is present. In some embodiments AA2 is present. In some embodiments AA3 is present. In some embodiments AA1 is A. In some embodiments AA2 is S. In some embodiments AA3 is D. In some embodiments AA3 is selected from any one amino acid C, D, E, N, P, and Q. In some embodiments AA3 is selected from D, E and G. In some embodiments AA3 is selected from E and G. In some embodiments AA3 is P. In some embodiments AA3 is C. In some embodiments AA4 is K. In some embodiments AA5 is P. In some embodiments AA6 is Y. In some embodiments AA7 is I. In some embodiments AA8 is L. In some embodiments the amino acid sequence of formula I, II, or III is not found in nature. In some embodiments the polypeptide comprising or consisting of the amino acid sequence of formula I, II, or III is not found in nature.

In some embodiments AA8 is the C-terminal amino acid. In some embodiments AA5 is P. In some embodiments AA6 is selected from Y and W. In some embodiments AA7 is selected from I and L.

In some embodiments AA2 when present is an amino acid selected from S, T, A, N, E and D. In some embodiments AA2 when present is an amino acid selected from S, T, G, A, N, E and D. In some embodiments AA5 is selected from P, S, D, A, T, K, and G. In some embodiments AA6 is selected from Y, N, I, and W. In some embodiments AA8 is selected from L, I, V, S, and M.

In some embodiments the polypeptide does not comprise any one of the sequences AVTEKKYILYDFSVTS (SEQ ID NO:5), PRRPYIL (SEQ ID NO:38), RRPYIL (SEQ ID NO:39), RPYIL (SEQ ID NO:40), RRPWIL (SEQ ID NO:41), KRPYIL (SEQ ID NO:42), KKPYIL (SEQ ID NO:43), Adamantoyl-KPYIL (SEQ ID NO:9), H-Lys-psi(CH₂NH)Lys-Pro-Tyr-Ile-Leu-OH (SEQ ID NO:44). In some embodiments the polypeptide does not comprise derivatives of Lys.

In some embodiments the polypeptide does not consists of any one of the sequences AVTEKKYILYDFSVTS, PRRPYIL, RRPYIL, RPYIL, RRPWIL, KRPYIL, KKPYIL, Adamantoyl-KPYIL, H-Lys-psi(CH₂NH)Lys-Pro-Tyr-Ile-Leu-OH. In some embodiments the polypeptide is not a derivative of KPYIL.

In some embodiments the amino acid sequence of formula I, II, or III only contains natural amino acids.

In some embodiments the first polypeptide of the invention is 5-50, such as 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, such as 5-18, such as 5-17, such as 5-16, such as 5-15, such as 5-14, such as 5-13, such as 5-12, such as 5-11, such as 5-10, such as 5-9, such as 5-8, such as 5-7, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments the first polypeptide of the invention is less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, such as 18, such as 17, such as 16, such as 15, such as 14, such as 13, such as 12, such as 11, such as 10, such as 9, such as 8, such as 7 amino acids in length.

In some embodiments the first polypeptide of the invention is 5-50, such as 6-50, such as 7-50, such as 8-50, such as 9-50, such as 10-50, such as 11-50, such as 12-50, such as 13-50, such as 14-50, such as 15-50, such as 16-50, such as 17-50, such as 18-50, such as 19-50, such as 20-50, such as 21-50, such as 22-50, such as 23-50, such as 24-50, such as 25-50, such as 26-50, such as 27-50, such as 28-50, such as 29-50, such as 30-50, such as 31-50, such as 32-50, such as 33-50, such as 34-50, such as 35-50, such as 36-50, such as 37-50, such as 38-50, such as 39-50, such as 40-50, such as 41-50, such as 42-50, such as 43-50, such as 44-50, such as 45-50, such as 46-50, such as 47-50, such as 48-50, such as 49-50 amino acids in length.

In some embodiments the first polypeptide of the invention is more than 5, such as 6, such as 7, such as 8, such as 9, such as 10, such as 11, such as 12, such as 13, such as 14, such as 15, such as 16, such as 17, such as 18, such as 19, such as 20, such as 21, such as 22, such as 23, such as 24, such as 25, such as 26, such as 27, such as 28, such as 29, such as 30, such as 31, such as 32, such as 33, such as 34, such as 35, such as 36, such as 37, such as 38, such as 39, such as 40, such as 41, such as 42, such as 43, such as 44, such as 45, such as 46, such as 47, such as 48, such as more than 49 amino acids in length.

In some embodiments the first polypeptide of the invention is an octapeptide or a heptapeptide

In some embodiments the first polypeptide of the invention has or comprises a sequence selected from ASDKPYIL, SDKPYIL, DKPYIL, and KPYIL.

In some embodiments the first polypeptide of the invention consist of or comprises a sequence selected from ASDKPYIL, AGDKNYIL, AGDKNYIT, AGDKSYIT, ADGKPYIV, AEDKDFIT, AADKPYIL, ATDKPYIL, AGDKPYIT, ASEKPYIL, ADGKPYVT, AGDKPYIL, ASDKPNIL, ASDKPYIT, AADKPFIL, ASDKAYIT, AGDKAYIT, ANGKPFIT, AGDKNFIT, ASDKSYIT, ASDKTYIT, ASDKNYIT, AGDKKYIT, AGDKNYIS, AADKNYIT, AGDKNYIM, AADKNFIM, AADKNFIT, and AGDKGIRS.

In some embodiments the first polypeptide of the invention is a polypeptide.

In some embodiments the first polypeptide of the invention is synthetically made.

In some embodiments the first polypeptide of the invention is a purified fragment.

In some embodiments the first polypeptide of the invention is purified from animal sources.

In some embodiments the first polypeptide of the invention is generated by enzymatic treatment of proteins from animal sources.

In some embodiments the first polypeptide of the invention has been modified by N terminal acylation or other chemical modifications to introduce protection groups.

In some specific embodiments, the first polypeptide of the invention consists of or comprises an amino acid sequence selected from the group consisting of KPYIL, KPYII (SEQ ID NO:45), KPYIV (SEQ ID NO:46), KPYLL (SEQ ID NO:47), KPYLI (SEQ ID NO:48), KPYLV (SEQ ID NO:49), KPYVL (SEQ ID NO:50), KPYVI (SEQ ID NO:51), KPYVV (SEQ ID NO:52), KPWIL (SEQ ID NO:53), KPWII (SEQ ID NO:54), KPWIV (SEQ ID NO:55), KPWLL (SEQ ID NO:56), KPWLI (SEQ ID NO:57), KPWLV (SEQ ID NO:58), KPWVL (SEQ ID NO:59), KPWVI (SEQ ID NO:60), KPWVV (SEQ ID NO:61), RPYIL (SEQ ID NO:40), RPYII (SEQ ID NO:62), RPYIV (SEQ ID NO:63), RPYLL (SEQ ID NO:64), RPYLI (SEQ ID NO:65), RPYLV (SEQ ID NO:66), RPYVL (SEQ ID NO:67), RPYVI (SEQ ID NO:68), RPYVV (SEQ ID NO:69), RPWIL (SEQ ID NO:70), RPWII (SEQ ID NO:71), RPWIV (SEQ ID NO:72), RPWLL (SEQ ID NO:73), RPWLI (SEQ ID NO:74), RPWLV (SEQ ID NO:75), RPWVL (SEQ ID NO:76), RPWVI (SEQ ID NO:77), and RPWVV (SEQ ID NO:78).

In some specific embodiments, the first polypeptide of the invention consist of or comprises an amino acid sequence selected from the group consisting of DKPYIL (SEQ ID NO:8), DKPYII (SEQ ID NO:79), DKPYIV (SEQ ID NO:80), DKPYLL (SEQ ID NO:81), DKPYLI (SEQ ID NO:82), DKPYLV (SEQ ID NO:83), DKPYVL (SEQ ID NO:84), DKPYVI (SEQ ID NO:85), DKPYVV (SEQ ID NO:86), DKPWIL (SEQ ID NO:87), DKPWII (SEQ ID NO:88), DKPWIV (SEQ ID NO:89), DKPWLL (SEQ ID NO:90), DKPWLI (SEQ ID NO:91), DKPWLV (SEQ ID NO:92), DKPWVL (SEQ ID NO:93), DKPWVI (SEQ ID NO:94), DKPWVV (SEQ ID NO:95), DRPYIL (SEQ ID NO:96), DRPYII (SEQ ID NO:97), DRPYIV (SEQ ID NO:98), DRPYLL (SEQ ID NO:99), DRPYLI (SEQ ID NO:100), DRPYLV (SEQ ID NO:101), DRPYVL (SEQ ID NO:102), DRPYVI (SEQ ID NO:103), DRPYVV (SEQ ID NO:104), DRPWIL (SEQ ID NO:105), DRPWII (SEQ ID NO:106), DRPWIV (SEQ ID NO:107), DRPWLL (SEQ ID NO:108), DRPWLI (SEQ ID NO:109), DRPWLV (SEQ ID NO:110), DRPWVL (SEQ ID NO:111), DRPWVI (SEQ ID NO:112), DRPWVV (SEQ ID NO:113), EKPYIL (SEQ ID NO:114), EKPYII (SEQ ID NO:115), EKPYIV (SEQ ID NO:116), EKPYLL (SEQ ID NO:117), EKPYLI (SEQ ID NO:118), EKPYLV (SEQ ID NO:119), EKPYVL (SEQ ID NO:120), EKPYVI (SEQ ID NO:121), EKPYVV (SEQ ID NO:122), EKPWIL (SEQ ID NO:123), EKPWII (SEQ ID NO:124), EKPWIV (SEQ ID NO:125), EKPWLL (SEQ ID NO:126), EKPWLI (SEQ ID NO:127), EKPWLV (SEQ ID NO:128), EKPWVL (SEQ ID NO:129), EKPWVI (SEQ ID NO:130), EKPWVV (SEQ ID NO:131), ERPYIL (SEQ ID NO:132), ERPYII (SEQ ID NO:133), ERPYIV (SEQ ID NO:134), ERPYLL (SEQ ID NO:135), ERPYLI (SEQ ID NO: 136), ERPYLV (SEQ ID NO:137), ERPYVL (SEQ ID NO:138), ERPYVI (SEQ ID NO:139), ERPYVV (SEQ ID NO:140), ERPWIL (SEQ ID NO:141), ERPWII (SEQ ID NO:142), ERPWIV (SEQ ID NO:143), ERPWLL (SEQ ID NO:144), ERPWLI (SEQ ID NO:145), ERPWLV (SEQ ID NO:146), ERPWVL (SEQ ID NO:147), ERPWVI (SEQ ID NO:148), ERPWVV (SEQ ID NO:149), RKPYIL (SEQ ID NO:150), RKPYII (SEQ ID NO:151), RKPYIV (SEQ ID NO:152), RKPYLL (SEQ ID NO:153), RKPYLI (SEQ ID NO:154), RKPYLV (SEQ ID NO:155), RKPYVL (SEQ ID NO:156), RKPYVI (SEQ ID NO:157), RKPYVV (SEQ ID NO:158), RKPWIL (SEQ ID NO:159), RKPWII (SEQ ID NO:160), RKPWIV (SEQ ID NO:161), RKPWLL (SEQ ID NO:162), RKPWLI (SEQ ID NO:163), RKPWLV (SEQ ID NO:164), RKPWVL (SEQ ID NO:165), RKPWVI (SEQ ID NO:166), RKPWVV (SEQ ID NO:167), RRPYIL (SEQ ID NO:39), RRPYII (SEQ ID NO:168), RRPYIV (SEQ ID NO:169), RRPYLL (SEQ ID NO:170), RRPYLI (SEQ ID NO:171), RRPYLV (SEQ ID NO:172), RRPYVL (SEQ ID NO:173), RRPYVI (SEQ ID NO:174), RRPYVV (SEQ ID NO:175), RRPWIL (SEQ ID NO:41), RRPWII (SEQ ID NO:176), RRPWIV (SEQ ID NO:177), RRPWLL (SEQ ID NO:178), RRPWLI (SEQ ID NO:179), RRPWLV (SEQ ID NO:180), RRPWVL (SEQ ID NO:181), RRPWVI (SEQ ID NO:182), RRPWVV (SEQ ID NO:183), GKPYIL (SEQ ID NO:184), GKPYII (SEQ ID NO:185), GKPYIV (SEQ ID NO:186), GKPYLL (SEQ ID NO:187), GKPYLI (SEQ ID NO:188), GKPYLV (SEQ ID NO:189), GKPYVL (SEQ ID NO:190), GKPYVI (SEQ ID NO:191), GKPYVV (SEQ ID NO:192), GKPWIL (SEQ ID NO:193), GKPWII (SEQ ID NO:194), GKPWIV (SEQ ID NO:195), GKPWLL (SEQ ID NO:196), GKPWLI (SEQ ID NO:197), GKPWLV (SEQ ID NO:198), GKPWVL (SEQ ID NO:199), GKPWVI (SEQ ID NO:200), GKPWVV (SEQ ID NO:201), GRPYIL (SEQ ID NO:202), GRPYII (SEQ ID NO:203), GRPYIV (SEQ ID NO:204), GRPYLL (SEQ ID NO:205), GRPYLI (SEQ ID NO:206), GRPYLV (SEQ ID NO:207), GRPYVL (SEQ ID NO:208), GRPYVI (SEQ ID NO:209), GRPYVV (SEQ ID NO:210), GRPWIL (SEQ ID NO:211), GRPWII (SEQ ID NO:212), GRPWIV (SEQ ID NO:213), GRPWLL (SEQ ID NO:214), GRPWLI (SEQ ID NO:215), GRPWLV (SEQ ID NO:216), GRPWVL (SEQ ID NO:217), GRPWVI (SEQ ID NO:218), and GRPWVV (SEQ ID NO:219).

In some specific embodiments, the first polypeptide of the invention consists of or comprises an amino acid sequence selected from the group consisting of SDKPYIL (SEQ ID NO:220), SDKPYII (SEQ ID NO:221), SDKPYIV (SEQ ID NO:222), SDKPYLL (SEQ ID NO:223), SDKPYLI (SEQ ID NO:224), SDKPYLV (SEQ ID NO:225), SDKPYVL (SEQ ID NO:226), SDKPYVI (SEQ ID NO:227), SDKPYVV (SEQ ID NO:228), SDKPWIL (SEQ ID NO:229), SDKPWII (SEQ ID NO:230), SDKPWIV (SEQ ID NO:231), SDKPWLL (SEQ ID NO:232), SDKPWLI (SEQ ID NO:233), SDKPWLV (SEQ ID NO:234), SDKPWVL (SEQ ID NO:235), SDKPWVI (SEQ ID NO:236), SDKPWVV (SEQ ID NO:237), SDRPYIL (SEQ ID NO:238), SDRPYII (SEQ ID NO:239), SDRPYIV (SEQ ID NO:240), SDRPYLL (SEQ ID NO:241), SDRPYLI (SEQ ID NO:242), SDRPYLV (SEQ ID NO:243), SDRPYVL (SEQ ID NO:244), SDRPYVI (SEQ ID NO:245), SDRPYVV (SEQ ID NO:246), SDRPWIL (SEQ ID NO:247), SDRPWII (SEQ ID NO:248), SDRPWIV (SEQ ID NO:249), SDRPWLL (SEQ ID NO:250), SDRPWLI (SEQ ID NO:251), SDRPWLV (SEQ ID NO:252), SDRPWVL (SEQ ID NO:253), SDRPWVI (SEQ ID NO:254), SDRPWVV (SEQ ID NO:255), SEKPYIL (SEQ ID NO:256), SEKPYII (SEQ ID NO:257), SEKPYIV (SEQ ID NO:258), SEKPYLL (SEQ ID NO:259), SEKPYLI (SEQ ID NO:260), SEKPYLV (SEQ ID NO:261), SEKPYVL (SEQ ID NO:262), SEKPYVI (SEQ ID NO:263), SEKPYVV (SEQ ID NO:264), SEKPWIL (SEQ ID NO:265), SEKPWII (SEQ ID NO:266), SEKPWIV (SEQ ID NO:267), SEKPWLL (SEQ ID NO:268), SEKPWLI (SEQ ID NO:269), SEKPWLV (SEQ ID NO:270), SEKPWVL (SEQ ID NO:271), SEKPWVI (SEQ ID NO:272), SEKPWVV (SEQ ID NO:273), SERPYIL (SEQ ID NO:274), SERPYII (SEQ ID NO:275), SERPYIV (SEQ ID NO:276), SERPYLL (SEQ ID NO:277), SERPYLI (SEQ ID NO:278), SERPYLV (SEQ ID NO:279), SERPYVL (SEQ ID NO:280), SERPYVI (SEQ ID NO:281), SERPYVV (SEQ ID NO:282), SERPWIL (SEQ ID NO:283), SERPWII (SEQ ID NO:284), SERPWIV (SEQ ID NO:285), SERPWLL (SEQ ID NO:286), SERPWLI (SEQ ID NO:287), SERPWLV (SEQ ID NO:288), SERPWVL (SEQ ID NO:289), SERPWVI (SEQ ID NO:290), SERPWVV (SEQ ID NO:291), TDKPYIL (SEQ ID NO:292), TDKPYII (SEQ ID NO:293), TDKPYIV (SEQ ID NO:294), TDKPYLL (SEQ ID NO:295), TDKPYLI (SEQ ID NO:296), TDKPYLV (SEQ ID NO:297), TDKPYVL (SEQ ID NO:298), TDKPYVI (SEQ ID NO:299), TDKPYVV (SEQ ID NO:300), TDKPWIL (SEQ ID NO:301), TDKPWII (SEQ ID NO:302), TDKPWIV (SEQ ID NO:303), TDKPWLL (SEQ ID NO:304), TDKPWLI (SEQ ID NO:305), TDKPWLV (SEQ ID NO:306), TDKPWVL (SEQ ID NO:307), TDKPWVI (SEQ ID NO:308), TDKPWVV (SEQ ID NO:309), TDRPYIL (SEQ ID NO:310), TDRPYII (SEQ ID NO:311), TDRPYIV (SEQ ID NO:312), TDRPYLL (SEQ ID NO:313), TDRPYLI (SEQ ID NO:314), TDRPYLV (SEQ ID NO:315), TDRPYVL (SEQ ID NO:316), TDRPYVI (SEQ ID NO:317), TDRPYVV (SEQ ID NO:318), TDRPWIL (SEQ ID NO:319), TDRPWII (SEQ ID NO:320), TDRPWIV (SEQ ID NO:321), TDRPWLL (SEQ ID NO:322), TDRPWLI (SEQ ID NO:323), TDRPWLV (SEQ ID NO:324), TDRPWVL (SEQ ID NO:325), TDRPWVI (SEQ ID NO:326), TDRPWVV (SEQ ID NO:327), TEKPYIL (SEQ ID NO:328), TEKPYII (SEQ ID NO:329), TEKPYIV (SEQ ID NO:330), TEKPYLL (SEQ ID NO:331), TEKPYLI (SEQ ID NO:332), TEKPYLV (SEQ ID NO:333), TEKPYVL (SEQ ID NO:334), TEKPYVI (SEQ ID NO:335), TEKPYVV (SEQ ID NO:336), TEKPWIL (SEQ ID NO:337), TEKPWII (SEQ ID NO:338), TEKPWIV (SEQ ID NO:339), TEKPWLL (SEQ ID NO:340), TEKPWLI (SEQ ID NO:341), TEKPWLV (SEQ ID NO:342), TEKPWVL (SEQ ID NO:343), TEKPWVI (SEQ ID NO:344), TEKPWVV (SEQ ID NO:345), TERPYIL (SEQ ID NO:346), TERPYII (SEQ ID NO:347), TERPYIV (SEQ ID NO:348), TERPYLL (SEQ ID NO:349), TERPYLI (SEQ ID NO:350), TERPYLV (SEQ ID NO:351), TERPYVL (SEQ ID NO:352), TERPYVI (SEQ ID NO:353), TERPYVV (SEQ ID NO:354), TERPWIL (SEQ ID NO:355), TERPWII (SEQ ID NO:356), TERPWIV (SEQ ID NO:357), TERPWLL (SEQ ID NO:358), TERPWLI (SEQ ID NO:359), TERPWLV (SEQ ID NO:360), TERPWVL (SEQ ID NO:361), TERPWVI (SEQ ID NO:362), and TERPWVV (SEQ ID NO:363).

In some specific embodiments, the first polypeptide of the invention consists of or comprises an amino acid sequence selected from the group consisting of ASDKPYII (SEQ ID NO:364), ASDKPYIV (SEQ ID NO:365), ASDKPYLL (SEQ ID NO:366), ASDKPYLI (SEQ ID NO:367), ASDKPYLV (SEQ ID NO:368), ASDKPYVL (SEQ ID NO:369), ASDKPYVI (SEQ ID NO:370), ASDKPYVV (SEQ ID NO:371), ASDKPWIL (SEQ ID NO:372), ASDKPWII (SEQ ID NO:373), ASDKPWIV (SEQ ID NO:374), ASDKPWLL (SEQ ID NO:375), ASDKPWLI (SEQ ID NO:376), ASDKPWLV (SEQ ID NO:377), ASDKPWVL (SEQ ID NO:378), ASDKPWVI (SEQ ID NO:379), ASDKPWVV (SEQ ID NO:380), ASDRPYIL (SEQ ID NO:381), ASDRPYII (SEQ ID NO:382), ASDRPYIV (SEQ ID NO:383), ASDRPYLL (SEQ ID NO:384), ASDRPYLI (SEQ ID NO:385), ASDRPYLV (SEQ ID NO:386), ASDRPYVL (SEQ ID NO:387), ASDRPYVI (SEQ ID NO:388), ASDRPYVV (SEQ ID NO:389), ASDRPWIL (SEQ ID NO:390), ASDRPWII (SEQ ID NO:391), ASDRPWIV (SEQ ID NO:392), ASDRPWLL (SEQ ID NO:393), ASDRPWLI (SEQ ID NO:394), ASDRPWLV (SEQ ID NO:395), ASDRPWVL (SEQ ID NO:396), ASDRPWVI (SEQ ID NO:397), ASDRPWVV (SEQ ID NO:398), ASEKPYIL (SEQ ID NO:399), ASEKPYII (SEQ ID NO:400), ASEKPYIV (SEQ ID NO:401), ASEKPYLL (SEQ ID NO:402), ASEKPYLI (SEQ ID NO:403), ASEKPYLV (SEQ ID NO:404), ASEKPYVL (SEQ ID NO:405), ASEKPYVI (SEQ ID NO:406), ASEKPYVV (SEQ ID NO:407), ASEKPWIL (SEQ ID NO:408), ASEKPWII (SEQ ID NO:409), ASEKPWIV (SEQ ID NO:410), ASEKPWLL (SEQ ID NO:411), ASEKPWLI (SEQ ID NO:412), ASEKPWLV (SEQ ID NO:413), ASEKPWVL (SEQ ID NO:414), ASEKPWVI (SEQ ID NO:415), ASEKPWVV (SEQ ID NO:416), ASERPYIL (SEQ ID NO:417), ASERPYII (SEQ ID NO:418), ASERPYIV (SEQ ID NO:419), ASERPYLL (SEQ ID NO:420), ASERPYLI (SEQ ID NO:421), ASERPYLV (SEQ ID NO:422), ASERPYVL (SEQ ID NO:423), ASERPYVI (SEQ ID NO:424), ASERPYVV (SEQ ID NO:425), ASERPWIL (SEQ ID NO:426), ASERPWII (SEQ ID NO:427), ASERPWIV (SEQ ID NO:428), ASERPWLL (SEQ ID NO:429), ASERPWLI (SEQ ID NO:430), ASERPWLV (SEQ ID NO:431), ASERPWVL (SEQ ID NO:432), ASERPWVI (SEQ ID NO:433), ASERPWVV (SEQ ID NO:434), ATDKPYIL (SEQ ID NO:435), ATDKPYII (SEQ ID NO:436), ATDKPYIV (SEQ ID NO:437), ATDKPYLL (SEQ ID NO:438), ATDKPYLI (SEQ ID NO:439), ATDKPYLV (SEQ ID NO:440), ATDKPYVL (SEQ ID NO:441), ATDKPYVI (SEQ ID NO:442), ATDKPYVV (SEQ ID NO:443), ATDKPWIL (SEQ ID NO:444), ATDKPWII (SEQ ID NO:445), ATDKPWIV (SEQ ID NO:446), ATDKPWLL (SEQ ID NO:447), ATDKPWLI (SEQ ID NO:448), ATDKPWLV (SEQ ID NO:449), ATDKPWVL (SEQ ID NO:450), ATDKPWVI (SEQ ID NO:451), ATDKPWVV (SEQ ID NO:452), ATDRPYIL (SEQ ID NO:453), ATDRPYII (SEQ ID NO:454), ATDRPYIV (SEQ ID NO:455), ATDRPYLL (SEQ ID NO:456), ATDRPYLI (SEQ ID NO:457), ATDRPYLV (SEQ ID NO:458), ATDRPYVL (SEQ ID NO:459), ATDRPYVI (SEQ ID NO:460), ATDRPYVV (SEQ ID NO:461), ATDRPWIL (SEQ ID NO:462), ATDRPWII (SEQ ID NO:463), ATDRPWIV (SEQ ID NO:464), ATDRPWLL (SEQ ID NO:465), ATDRPWLI (SEQ ID NO:466), ATDRPWLV (SEQ ID NO:467), ATDRPWVL (SEQ ID NO:468), ATDRPWVI (SEQ ID NO:469), ATDRPWVV (SEQ ID NO:470), ATEKPYIL (SEQ ID NO:471), ATEKPYII (SEQ ID NO:472), ATEKPYIV (SEQ ID NO:473), ATEKPYLL (SEQ ID NO:474), ATEKPYLI (SEQ ID NO:475), ATEKPYLV (SEQ ID NO:476), ATEKPYVL (SEQ ID NO:477), ATEKPYVI (SEQ ID NO:478), ATEKPYVV (SEQ ID NO:479), ATEKPWIL (SEQ ID NO:480), ATEKPWII (SEQ ID NO:481), ATEKPWIV (SEQ ID NO:482), ATEKPWLL (SEQ ID NO:483), ATEKPWLI (SEQ ID NO:484), ATEKPWLV (SEQ ID NO:485), ATEKPWVL (SEQ ID NO:486), ATEKPWVI (SEQ ID NO:487), ATEKPWVV (SEQ ID NO:488), ATERPYIL (SEQ ID NO:489), ATERPYII (SEQ ID NO:490), ATERPYIV (SEQ ID NO:491), ATERPYLL (SEQ ID NO:492), ATERPYLI (SEQ ID NO:493), ATERPYLV (SEQ ID NO:494), ATERPYVL (SEQ ID NO:495), ATERPYVI (SEQ ID NO:496), ATERPYVV (SEQ ID NO:497), ATERPWIL (SEQ ID NO:498), ATERPWII (SEQ ID NO:499), ATERPWIV (SEQ ID NO:500), ATERPWLL (SEQ ID NO:501), ATERPWLI (SEQ ID NO:502), ATERPWLV (SEQ ID NO:503), ATERPWVL (SEQ ID NO:504), ATERPWVI (SEQ ID NO:505), ATERPWVV (SEQ ID NO:506), LSDKPYIL (SEQ ID NO:507), LSDKPYII (SEQ ID NO:508), LSDKPYIV (SEQ ID NO:509), LSDKPYLL (SEQ ID NO:510), LSDKPYLI (SEQ ID NO:511), LSDKPYLV (SEQ ID NO:512), LSDKPYVL (SEQ ID NO:513), LSDKPYVI (SEQ ID NO:514), LSDKPYVV (SEQ ID NO:515), LSDKPWIL (SEQ ID NO:516), LSDKPWII (SEQ ID NO:517), LSDKPWIV (SEQ ID NO:518), LSDKPWLL (SEQ ID NO:519), LSDKPWLI (SEQ ID NO:520), LSDKPWLV (SEQ ID NO:521), LSDKPWVL (SEQ ID NO:522), LSDKPWVI (SEQ ID NO:523), LSDKPWVV (SEQ ID NO:524), LSDRPYIL (SEQ ID NO:525), LSDRPYII (SEQ ID NO:526), LSDRPYIV (SEQ ID NO:527), LSDRPYLL (SEQ ID NO:528), LSDRPYLI (SEQ ID NO:529), LSDRPYLV (SEQ ID NO:530), LSDRPYVL (SEQ ID NO:531), LSDRPYVI (SEQ ID NO:532), LSDRPYVV (SEQ ID NO:533), LSDRPWIL (SEQ ID NO:534), LSDRPWII (SEQ ID NO:535), LSDRPWIV (SEQ ID NO:536), LSDRPWLL (SEQ ID NO:537), LSDRPWLI (SEQ ID NO:538), LSDRPWLV (SEQ ID NO:539), LSDRPWVL (SEQ ID NO:540), LSDRPWVI (SEQ ID NO:541), LSDRPWVV (SEQ ID NO:542), LSEKPYIL (SEQ ID NO:543), LSEKPYII (SEQ ID NO:544), LSEKPYIV (SEQ ID NO:545), LSEKPYLL (SEQ ID NO:546), LSEKPYLI (SEQ ID NO:547), LSEKPYLV (SEQ ID NO:548), LSEKPYVL (SEQ ID NO:549), LSEKPYVI (SEQ ID NO:550), LSEKPYVV (SEQ ID NO:551), LSEKPWIL (SEQ ID NO:552), LSEKPWII (SEQ ID NO:553), LSEKPWIV (SEQ ID NO:554), LSEKPWLL (SEQ ID NO:555), LSEKPWLI (SEQ ID NO:556), LSEKPWLV (SEQ ID NO:557), LSEKPWVL (SEQ ID NO:558), LSEKPWVI (SEQ ID NO:559), LSEKPWVV (SEQ ID NO:560), LSERPYIL (SEQ ID NO:561), LSERPYII (SEQ ID NO:562), LSERPYIV (SEQ ID NO:563), LSERPYLL (SEQ ID NO:564), LSERPYLI (SEQ ID NO:565), LSERPYLV (SEQ ID NO:566), LSERPYVL (SEQ ID NO:567), LSERPYVI (SEQ ID NO:568), LSERPYVV (SEQ ID NO:569), LSERPWIL (SEQ ID NO:570), LSERPWII (SEQ ID NO:571), LSERPWIV (SEQ ID NO:572), LSERPWLL (SEQ ID NO:573), LSERPWLI (SEQ ID NO:574), LSERPWLV (SEQ ID NO:575), LSERPWVL (SEQ ID NO:576), LSERPWVI (SEQ ID NO:577), LSERPWVV (SEQ ID NO:578), LTDKPYIL (SEQ ID NO:579), LTDKPYII (SEQ ID NO:580), LTDKPYIV (SEQ ID NO:581), LTDKPYLL (SEQ ID NO:582), LTDKPYLI (SEQ ID NO:583), LTDKPYLV (SEQ ID NO:584), LTDKPYVL (SEQ ID NO:585), LTDKPYVI (SEQ ID NO:586), LTDKPYVV (SEQ ID NO:587), LTDKPWIL (SEQ ID NO:588), LTDKPWII (SEQ ID NO:589), LTDKPWIV (SEQ ID NO:590), LTDKPWLL (SEQ ID NO:591), LTDKPWLI (SEQ ID NO:592), LTDKPWLV (SEQ ID NO:593), LTDKPWVL (SEQ ID NO:594), LTDKPWVI (SEQ ID NO:595), LTDKPWVV (SEQ ID NO:596), LTDRPYIL (SEQ ID NO:597), LTDRPYII (SEQ ID NO:598), LTDRPYIV (SEQ ID NO:599), LTDRPYLL (SEQ ID NO:600), LTDRPYLI (SEQ ID NO:601), LTDRPYLV (SEQ ID NO:602), LTDRPYVL (SEQ ID NO:603), LTDRPYVI (SEQ ID NO:604), LTDRPYVV (SEQ ID NO:605), LTDRPWIL (SEQ ID NO:606), LTDRPWII (SEQ ID NO:607), LTDRPWIV (SEQ ID NO:608), LTDRPWLL (SEQ ID NO:609), LTDRPWLI (SEQ ID NO:610), LTDRPWLV (SEQ ID NO:611), LTDRPWVL (SEQ ID NO:612), LTDRPWVI (SEQ ID NO:613), LTDRPWVV (SEQ ID NO:614), LTEKPYIL (SEQ ID NO:615), LTEKPYII (SEQ ID NO:616), LTEKPYIV (SEQ ID NO:617), LTEKPYLL (SEQ ID NO:618), LTEKPYLI (SEQ ID NO:619), LTEKPYLV (SEQ ID NO:620), LTEKPYVL (SEQ ID NO:621), LTEKPYVI (SEQ ID NO:622), LTEKPYVV (SEQ ID NO:623), LTEKPWIL (SEQ ID NO:624), LTEKPWII (SEQ ID NO:625), LTEKPWIV (SEQ ID NO:626), LTEKPWLL (SEQ ID NO:627), LTEKPWLI (SEQ ID NO:628), LTEKPWLV (SEQ ID NO:629), LTEKPWVL (SEQ ID NO:630), LTEKPWVI (SEQ ID NO:631), LTEKPWVV (SEQ ID NO:632), LTERPYIL (SEQ ID NO:633), LTERPYII (SEQ ID NO:634), LTERPYIV (SEQ ID NO:635), LTERPYLL (SEQ ID NO:636), LTERPYLI (SEQ ID NO:637), LTERPYLV (SEQ ID NO:638), LTERPYVL (SEQ ID NO:639), LTERPYVI (SEQ ID NO:640), LTERPYVV (SEQ ID NO:641), LTERPWIL (SEQ ID NO:642), LTERPWII (SEQ ID NO:643), LTERPWIV (SEQ ID NO:644), LTERPWLL (SEQ ID NO:645), LTERPWLI (SEQ ID NO:646), LTERPWLV (SEQ ID NO:647), LTERPWVL (SEQ ID NO:648), LTERPWVI (SEQ ID NO:649), LTERPWVV (SEQ ID NO:650), ISDKPYIL (SEQ ID NO:651), ISDKPYII (SEQ ID NO:652), ISDKPYIV (SEQ ID NO:653), ISDKPYLL (SEQ ID NO:654), ISDKPYLI (SEQ ID NO:655), ISDKPYLV (SEQ ID NO:656), ISDKPYVL (SEQ ID NO:657), ISDKPYVI (SEQ ID NO:658), ISDKPYVV (SEQ ID NO:659), ISDKPWIL (SEQ ID NO:660), ISDKPWII (SEQ ID NO:661), ISDKPWIV (SEQ ID NO:662), ISDKPWLL (SEQ ID NO:663), ISDKPWLI (SEQ ID NO:664), ISDKPWLV (SEQ ID NO:665), ISDKPWVL (SEQ ID NO:666), ISDKPWVI (SEQ ID NO:667), ISDKPWVV (SEQ ID NO:668), ISDRPYIL (SEQ ID NO:669), ISDRPYII (SEQ ID NO:670), ISDRPYIV (SEQ ID NO:671), ISDRPYLL (SEQ ID NO:672), ISDRPYLI (SEQ ID NO:673), ISDRPYLV (SEQ ID NO:674), ISDRPYVL (SEQ ID NO:675), ISDRPYVI (SEQ ID NO:676), ISDRPYVV (SEQ ID NO:677), ISDRPWIL (SEQ ID NO:678), ISDRPWII (SEQ ID NO:679), ISDRPWIV (SEQ ID NO:680), ISDRPWLL (SEQ ID NO:681), ISDRPWLI (SEQ ID NO:682), ISDRPWLV (SEQ ID NO:683), ISDRPWVL (SEQ ID NO:684), ISDRPWVI (SEQ ID NO:685), ISDRPWVV (SEQ ID NO:686), ISEKPYIL (SEQ ID NO:687), ISEKPYII (SEQ ID NO:688), ISEKPYIV (SEQ ID NO:689), ISEKPYLL (SEQ ID NO:690), ISEKPYLI (SEQ ID NO:691), ISEKPYLV (SEQ ID NO:692), ISEKPYVL (SEQ ID NO:693), ISEKPYVI (SEQ ID NO:694), ISEKPYVV (SEQ ID NO:695), ISEKPWIL (SEQ ID NO:696), ISEKPWII (SEQ ID NO:697), ISEKPWIV (SEQ ID NO:698), ISEKPWLL (SEQ ID NO:699), ISEKPWLI (SEQ ID NO:700), ISEKPWLV (SEQ ID NO:701), ISEKPWVL (SEQ ID NO:702), ISEKPWVI (SEQ ID NO:703), ISEKPWVV (SEQ ID NO:704), ISERPYIL (SEQ ID NO:705), ISERPYII (SEQ ID NO:706), ISERPYIV (SEQ ID NO:707), ISERPYLL (SEQ ID NO:708), ISERPYLI (SEQ ID NO:709), ISERPYLV (SEQ ID NO:710), ISERPYVL (SEQ ID NO:711), ISERPYVI (SEQ ID NO:712), ISERPYVV (SEQ ID NO:713), ISERPWIL (SEQ ID NO:714), ISERPWII (SEQ ID NO:715), ISERPWIV (SEQ ID NO:716), ISERPWLL (SEQ ID NO:717), ISERPWLI (SEQ ID NO:718), ISERPWLV (SEQ ID NO:719), ISERPWVL (SEQ ID NO:720), ISERPWVI (SEQ ID NO:721), ISERPWVV (SEQ ID NO:722), ITDKPYIL (SEQ ID NO:723), ITDKPYII (SEQ ID NO:724), ITDKPYIV (SEQ ID NO:725), ITDKPYLL (SEQ ID NO:726), ITDKPYLI (SEQ ID NO:727), ITDKPYLV (SEQ ID NO:728), ITDKPYVL (SEQ ID NO:729), ITDKPYVI (SEQ ID NO:730), ITDKPYVV (SEQ ID NO:731), ITDKPWIL (SEQ ID NO:732), ITDKPWII (SEQ ID NO:733), ITDKPWIV (SEQ ID NO:734), ITDKPWLL (SEQ ID NO:735), ITDKPWLI (SEQ ID NO:736), ITDKPWLV (SEQ ID NO:737), ITDKPWVL (SEQ ID NO:738), ITDKPWVI (SEQ ID NO:739), ITDKPWVV (SEQ ID NO:740), ITDRPYIL (SEQ ID NO:741), ITDRPYII (SEQ ID NO:742), ITDRPYIV (SEQ ID NO:743), ITDRPYLL (SEQ ID NO:744), ITDRPYLI (SEQ ID NO:745), ITDRPYLV (SEQ ID NO:746), ITDRPYVL (SEQ ID NO:747), ITDRPYVI (SEQ ID NO:748), ITDRPYVV (SEQ ID NO:749), ITDRPWIL (SEQ ID NO:750), ITDRPWII (SEQ ID NO:751), ITDRPWIV (SEQ ID NO:752), ITDRPWLL (SEQ ID NO:753), ITDRPWLI (SEQ ID NO:754), ITDRPWLV (SEQ ID NO:755), ITDRPWVL (SEQ ID NO:756), ITDRPWVI (SEQ ID NO:757), ITDRPWVV (SEQ ID NO:758), ITEKPYIL (SEQ ID NO:759), ITEKPYII (SEQ ID NO:760), ITEKPYIV (SEQ ID NO:761), ITEKPYLL (SEQ ID NO:762), ITEKPYLI (SEQ ID NO:763), ITEKPYLV (SEQ ID NO:764), ITEKPYVL (SEQ ID NO:765), ITEKPYVI (SEQ ID NO:766), ITEKPYVV (SEQ ID NO:767), ITEKPWIL (SEQ ID NO:768), ITEKPWII (SEQ ID NO:769), ITEKPWIV (SEQ ID NO:770), ITEKPWLL (SEQ ID NO:771), ITEKPWLI (SEQ ID NO:772), ITEKPWLV (SEQ ID NO:773), ITEKPWVL (SEQ ID NO:774), ITEKPWVI (SEQ ID NO:775), ITEKPWVV (SEQ ID NO:776), ITERPYIL (SEQ ID NO:777), ITERPYII (SEQ ID NO:778), ITERPYIV (SEQ ID NO:779), ITERPYLL (SEQ ID NO:780), ITERPYLI (SEQ ID NO:781), ITERPYLV (SEQ ID NO:782), ITERPYVL (SEQ ID NO:783), ITERPYVI (SEQ ID NO:784), ITERPYVV (SEQ ID NO:785), ITERPWIL (SEQ ID NO:786), ITERPWII (SEQ ID NO:787), ITERPWIV (SEQ ID NO:788), ITERPWLL (SEQ ID NO:789), ITERPWLI (SEQ ID NO:790), ITERPWLV (SEQ ID NO:791), ITERPWVL (SEQ ID NO:792), ITERPWVI (SEQ ID NO:793), ITERPWVV (SEQ ID NO:794), VSDKPYIL (SEQ ID NO:795), VSDKPYII (SEQ ID NO:796), VSDKPYIV (SEQ ID NO:797), VSDKPYLL (SEQ ID NO:798), VSDKPYLI (SEQ ID NO:799), VSDKPYLV (SEQ ID NO:800), VSDKPYVL (SEQ ID NO:801), VSDKPYVI (SEQ ID NO:802), VSDKPYVV (SEQ ID NO:803), VSDKPWIL (SEQ ID NO:804), VSDKPWII (SEQ ID NO:805), VSDKPWIV (SEQ ID NO:806), VSDKPWLL (SEQ ID NO:807), VSDKPWLI (SEQ ID NO:808), VSDKPWLV (SEQ ID NO:809), VSDKPWVL (SEQ ID NO:810), VSDKPWVI (SEQ ID NO:811), VSDKPWVV (SEQ ID NO:812), VSDRPYIL (SEQ ID NO:813), VSDRPYII (SEQ ID NO:814), VSDRPYIV (SEQ ID NO:815), VSDRPYLL (SEQ ID NO:816), VSDRPYLI (SEQ ID NO:817), VSDRPYLV (SEQ ID NO:818), VSDRPYVL (SEQ ID NO:819), VSDRPYVI (SEQ ID NO:820), VSDRPYVV (SEQ ID NO:821), VSDRPWIL (SEQ ID NO:822), VSDRPWII (SEQ ID NO:823), VSDRPWIV (SEQ ID NO:824), VSDRPWLL (SEQ ID NO:825), VSDRPWLI (SEQ ID NO:826), VSDRPWLV (SEQ ID NO:827), VSDRPWVL (SEQ ID NO:828), VSDRPWVI (SEQ ID NO:829), VSDRPWVV (SEQ ID NO:830), VSEKPYIL (SEQ ID NO:831), VSEKPYII (SEQ ID NO:832), VSEKPYIV (SEQ ID NO:833), VSEKPYLL (SEQ ID NO:834), VSEKPYLI (SEQ ID NO:835), VSEKPYLV (SEQ ID NO:836), VSEKPYVL (SEQ ID NO:837), VSEKPYVI (SEQ ID NO:838), VSEKPYVV (SEQ ID NO:839), VSEKPWIL (SEQ ID NO:840), VSEKPWII (SEQ ID NO:841), VSEKPWIV (SEQ ID NO:842), VSEKPWLL (SEQ ID NO:843), VSEKPWLI (SEQ ID NO:844), VSEKPWLV (SEQ ID NO:845), VSEKPWVL (SEQ ID NO:846), VSEKPWVI (SEQ ID NO:847), VSEKPWVV (SEQ ID NO:848), VSERPYIL (SEQ ID NO:849), VSERPYII (SEQ ID NO:850), VSERPYIV (SEQ ID NO:851), VSERPYLL (SEQ ID NO:852), VSERPYLI (SEQ ID NO:853), VSERPYLV (SEQ ID NO:854), VSERPYVL (SEQ ID NO:855), VSERPYVI (SEQ ID NO:856), VSERPYVV (SEQ ID NO:857), VSERPWIL (SEQ ID NO:858), VSERPWII (SEQ ID NO:859), VSERPWIV (SEQ ID NO:860), VSERPWLL (SEQ ID NO:861), VSERPWLI (SEQ ID NO:862), VSERPWLV (SEQ ID NO:863), VSERPWVL (SEQ ID NO:864), VSERPWVI (SEQ ID NO:865), VSERPWVV (SEQ ID NO:866), VTDKPYIL (SEQ ID NO:867), VTDKPYII (SEQ ID NO:868), VTDKPYIV (SEQ ID NO:869), VTDKPYLL (SEQ ID NO:870), VTDKPYLI (SEQ ID NO:871), VTDKPYLV (SEQ ID NO:872), VTDKPYVL (SEQ ID NO:873), VTDKPYVI (SEQ ID NO:874), VTDKPYVV (SEQ ID NO:875), VTDKPWIL (SEQ ID NO:876), VTDKPWII (SEQ ID NO:877), VTDKPWIV (SEQ ID NO:878), VTDKPWLL (SEQ ID NO:879), VTDKPWLI (SEQ ID NO:880), VTDKPWLV (SEQ ID NO:881), VTDKPWVL (SEQ ID NO:882), VTDKPWVI (SEQ ID NO:883), VTDKPWVV (SEQ ID NO:884), VTDRPYIL (SEQ ID NO:885), VTDRPYII (SEQ ID NO:886), VTDRPYIV (SEQ ID NO:887), VTDRPYLL (SEQ ID NO:888), VTDRPYLI (SEQ ID NO:889), VTDRPYLV (SEQ ID NO:890), VTDRPYVL (SEQ ID NO:891), VTDRPYVI (SEQ ID NO:892), VTDRPYVV (SEQ ID NO:893), VTDRPWIL (SEQ ID NO:894), VTDRPWII (SEQ ID NO:895), VTDRPWIV (SEQ ID NO:896), VTDRPWLL (SEQ ID NO:897), VTDRPWLI (SEQ ID NO:898), VTDRPWLV (SEQ ID NO:899), VTDRPWVL (SEQ ID NO:900), VTDRPWVI (SEQ ID NO:901), VTDRPWVV (SEQ ID NO:902), VTEKPYIL (SEQ ID NO:903), VTEKPYII (SEQ ID NO:904), VTEKPYIV (SEQ ID NO:905), VTEKPYLL (SEQ ID NO:906), VTEKPYLI (SEQ ID NO:907), VTEKPYLV (SEQ ID NO:908), VTEKPYVL (SEQ ID NO:909), VTEKPYVI (SEQ ID NO:910), VTEKPYVV (SEQ ID NO:911), VTEKPWIL (SEQ ID NO:912), VTEKPWII (SEQ ID NO:913), VTEKPWIV (SEQ ID NO:914), VTEKPWLL (SEQ ID NO:915), VTEKPWLI (SEQ ID NO:916), VTEKPWLV (SEQ ID NO:917), VTEKPWVL (SEQ ID NO:918), VTEKPWVI (SEQ ID NO:919), VTEKPWVV (SEQ ID NO:920), VTERPYIL (SEQ ID NO:921), VTERPYII (SEQ ID NO:922), VTERPYIV (SEQ ID NO:923), VTERPYLL (SEQ ID NO:924), VTERPYLI (SEQ ID NO:925), VTERPYLV (SEQ ID NO:926), VTERPYVL (SEQ ID NO:927), VTERPYVI (SEQ ID NO:928), VTERPYVV (SEQ ID NO:929), VTERPWIL (SEQ ID NO:930), VTERPWII (SEQ ID NO:931), VTERPWIV (SEQ ID NO:932), VTERPWLL (SEQ ID NO:933), VTERPWLI (SEQ ID NO:934), VTERPWLV (SEQ ID NO:935), VTERPWVL (SEQ ID NO:936), VTERPWVI (SEQ ID NO:937), and VTERPWVV (SEQ ID NO:938).

In some specific embodiments, the first polypeptide of the invention consists of or comprises an amino acid sequence derived from Alpha-actinin-1, such as a sequence selected from ASDKPYIL, AGDKNYIL, AGDKNYIT, AGDKSYIT, ADGKPYIV, and AEDKDFIT.

In some specific embodiments, the first polypeptide of the invention consists of or comprises an amino acid sequence derived from Alpha-actinin-2, such as a sequence selected from ASDKPYIL, AADKPYIL, AGDKNYIT, ATDKPYIL, AGDKPYIT, ASEKPYIL, ADGKPYVT, AGDKPYIL, ASDKPNIL, ASDKPYIT, AADKPFIL, ASDKAYIT, AGDKAYIT, ANGKPFIT, and AGDKNFIT.

In some specific embodiments, the first polypeptide of the invention consists of or comprises an amino acid sequence derived from Alpha-actinin-3, such as a sequence selected from ASDKPYIL, AADKPYIL, ASDKAYIT, ASDKSYIT, ASDKTYIT, ASDKNYIT, AGDKNYIL, AGDKSYIT, AGDKNYIT, AGDKKYIT, and AGDKNYIS.

In some specific embodiments, the first polypeptide of the invention consists of or comprises an amino acid sequence derived from Alpha-actinin-4, such as a sequence selected from ASDKPYIL, AGDKPYIL, AADKNYIT, AGDKNYIM, AGDKNYIT, AADKNFIM, AADKNFIT, AGDKGIRS, and AGDKNFIT.

In some embodiments, the second peptide hormone of the invention is selected from the list consisting of Cholecystokinin (CCK), Gastrin, Secretin, Vasoactive Intestinal Peptide (VIP), Glucose-dependent insulinotropic peptide (GIP), Glucagon-like Peptide 1 and 2 (GLP-1 and -2), Bombesin, Chromogranin A, Glucagon, Insulin, Leptin, Neuropeptide Y, Neurotensin, Neuromedin, Pancreatic Polypeptide, PYY, Amylin, Oxyntomodulin, Xexin, Motilin, Grehlin, and Somatostatin, and bioactive analogues or variants of any one of these peptide hormones.

In some embodiments, the second peptide hormone of the invention is a polypeptide being a GLP-1 receptor agonist is selected from the group consisting of: native human GLP-1(7-37); native human GLP-1(7-36) amide; Exenatide; Exenatide LAR; Liraglutide; Semaglutide; Taspoglutide; Albiglutide; Lixisenatide; Dulaglutide, and Oxyntomodulin.

The present invention further relates to compositions comprising a combination of the first and a second peptide hormone of the invention. In some embodiments the compositions of the invention is capable of promoting satiety or for reducing feed intake in a subject upon consumption.

In some embodiments in the compositions of the invention the amount of the first polypeptide in the composition is less than about 10 g, such as less than 9 g, 8 g, 7 g, 6 g, 5 g, 4 g, 3 g, 2 g, 1 g, 900 mg, 800 mg, 700 mg, 600 mg, 500 mg, 400 mg, 300 mg, 200 mg, 150 mg, 100 mg, 90 mg, 80 mg, 70 mg, 60 mg, 50 mg, 40 mg, 30 mg, 25 mg, 20 mg, 15 mg, 10 mg, or 5 mg.

In some embodiments in the compositions of the invention the amount of the first polypeptide in the composition is at least about 5 mg, such as at least about 10 mg, 15 mg, 20 mg, 25 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 800 mg, 900 mg, 1 g, 2 g, 3 g, 4 g, 5 g, 6 g, 7 g, 8 g, 9 g, or 10 g.

In some embodiments in the compositions of the invention the energy content derived through the process of cellular respiration is less than 50 kilojoules (kJ), such as less than 40 kJ, such as less than 30 kJ, such as less than 20 kJ, such as less than 10 kJ, such as less than 5000 Joules (J), such as less than 1000 J, such as less than 900 J, such as less than 800 J, such as less than 700 J, such as less than 600 J, such as less than 500 J, such as less than 400 J, such as less than 300 J, such as less than 200 J, such as less than 100 J, such as less than 50 J.

In some embodiments the compositions of the invention is a food composition.

In some embodiments the compositions of the invention is a fermented composition.

In some embodiments the compositions of the invention is a dairy product.

In some embodiments the compositions of the invention is a pharmaceutical composition.

In some embodiments the compositions of the invention is a nutritional composition.

In some embodiments the compositions of the invention is an oral dosage form. In some embodiments the oral dosage form is selected from the group comprising tablets, capsules, caplets, slurries, sachets, suspensions, chewing gum, and powder formulation that may be dissolved in a liquid. In some embodiments the oral dosage form is a suspension. In some embodiments the oral dosage form is a powder formulation that may be dissolved in a liquid. In some embodiments the liquid is water, milk, juice, or yogurt.

### Example 1

Assays:

Ca²⁺ Flux Assay/Calcium Response Assay:

Elevation of intracellular calcium level was measured using the fluorescent calcium chelating dye Fluo-4 AM (ThermoFischer Scientific, Denmark). Briefly, cells were grown as a monolayer in 96-well tissue culture plates (Sarstedt, Germany) to near confluence in appropriate growth medium as described in the cell culture section. Prior to the start of the assay, the cells were incubated with 1.5 μM Fluo-4 AM in complete culture media mixed 1:1 with Hank's balanced salt solution (HBSS, ThermoFischer Scientific, Denmark) containing 25 mM HEPES (pH 7.4), 1% BSA (Sigma-Aldrich, Denmark), 2% ink (Soluro GMBH, Germany), 0.01% Pluronic F-127 (Sigma-Aldrich, Denmark) and 1 mM Probenecide (Sigma-Aldrich) for 60 minutes at 37° C.

All test compounds were dissolved in water, and then diluted in 1×HBSS containing 25 mM HEPES (pH 7.4), 1% BSA and 2% ink. Without any removal of excess Fluo-4 AM, test compounds were added directly into the wells and fluorescence were measured using instrument settings for excitation at 488 nm and emission at 525 nm in a microtiter plate reader (SpectraMax M5, Molecular Devices, USA).

Cell Culture:

Cell culture media, Dulbecco's phosphate-buffered saline, pH 7.4 (DPBS), glutamine, trypsin-EDTA and antibiotics were obtained from ThermoFischer Scientific (Denmark). Fetal bovine serum and all other chemicals were purchased from Sigma-Aldrich (Denmark), unless otherwise stated.

Murine intestinal enteroendocrine L-cell lines that expresses the proglucagon gene and secretes GLP-1 in vitro were used. Cells were grown in DMEM containing 1 g/L D-glucose, 10% fetal bovine serum, 2 mM glutamine, 1% penicillin/streptomycin/neomycin and cultured in a humidified incubator in 95% air and 5% CO2 at 37° C.

Other murine intestinal cell lines were cultured in Dulbecco's modified Eagle's medium (DMEM) supplemented with 2 mM glutamine, 2.5 g/L glucose, 20 mM HEPES, 60 nM sodium selenite, 5 μg/ml transferrin, 5 μg/ml insulin, 50 nM dexamethasone, 10 nM EGF, 1 nM triiodothyronine, 2% fetal bovine serum and 1% penicillin/streptomycin/neomycin at 37° C. in 5% CO2-95% air atmosphere.

Human intestinal cell lines were cultured in McCoy's modified 5A medium supplemented with 10% fetal bovine serum and 1% penicillin/streptomycin/neomycin at 37° C. and 5% CO2 in a humidified incubator.

Cells were routinely sub-cultivated 1:3 and given new media every second day.

Determination of GLP-1 Levels:

GLP-1 levels were determined using a sandwich enzyme-linked immunoabsorbant assay (ELISA). The primary antibody to GLP-1 [2.5 μg/ml mouse monoclonal (HYB 147-06) in 0.05M bicarbonate/carbonate buffer; BioPorto Diagnostics A/S, Gentofte, Denmark) was coated on a flat-bottom 96-well plate (Sarstedt, Nümbrecht, Germany) for at least 24 hours at 4° C. This primary antibody is specific for the amidated C-terminus of the peptide and reacts with GLP-1 (7-36), GLP-1 (9-36) and GLP-1 (1-36), but not with GLP-1 (7-37). After blocking the plate using a PBS buffer containing 4% w/v BSA (Sigma-Aldrich, Denmark) and 0.1% v/v Tween 20 (Sigma-Aldrich) for 1 hour at room temperature, the plate was washed four times with PBS buffer containing 0.1% v/v Tween 20. A standard curve with GLP-1 peptide [human GLP-1 (7-36), Sigma-Aldrich, Denmark) concentrations ranging from 0 pg/ml to 1000 pg/ml was prepared in PBS buffer containing 0.5% BSA and 0.05% Tween 20, and samples were diluted if necessary. Samples and standards were added to the microtiter plate and incubated with the primary antibody for two hours at room temperature. Subsequently, the plate was washed four times, and the wells were incubated with a secondary biotinylated antibody to GLP-1 [1 μg/ml; mouse monoclonal (ABS 033-01), BioPorto Diagnostics A/S, Gentofte, Denmark) for two hours at room temperature. After another washing step, samples were incubated with streptavidin-horseradish peroxidase (1:200, Dako A/S, Denmark) for 45 minutes followed by an incubation with TMB solution (containing 3,3′,5,′5-tetramethylbenzidine and H2O2, SMS-gruppen, Denmark). The reaction was stopped by adding H2SO4 (0.2M), and the absorbance of the yellow end product was measured at 450 nm on a microtiter plate spectrophotometer (Spectra Max M5, Molecular Devices, USA). The concentrations of the samples were determined by interpolation to the concentrations of the standard solutions.

Cells (˜5×10\hat{ }5 per sample) were incubated for up to 90 min in Dulbeccos Modified Eagle Medium (DMEM) containing 5.56 mM glucose in absence or presence of different amounts (weight/volume) of protein hydrolysate (pig heart). Supernatant was filtered through 0.45 micron filters and assayed for content of GLP-1 as described in ELISA protocol. Data are mean+SEM from quadruplicate samples.

Preparation of Bioactive Peptides by Enzymatic Digestion of Meat

Minced meat is diluted 1-10 times with distilled water, adjusted to pH 1-3 with hydrochloric acid, and incubated with 0.01-10% pepsin (w/w) at 4-40° C. for ½-12 h with adequate mixing. Insoluble material is removed by centrifugation at 100-1000 xg for 3-30 min, and supernatant is neutralized with NaOH. Using sterile conditions, low molecular weight peptides in supernatant are recovered by tangential ultrafiltration at 4-40° C. for ½-12 h, and excess water is evaporated at 25-50° C. for up to 12 h. The concentrated dialysate is tested for bioactivity with cells and used for further purification by HPLC.

Purification and Identification of Bioactive Peptide ASDKPYIL

Upconcentrated dialysates were fractionated on preparative C18 columns using buffer B: 20 mM phosphate buffer pH 8.25/10% ACN and a gradient of 0-40% in buffer A: 60% ACN in same buffer. Fractions were tested for bioactivity and further purified by isocratic elution using EVO C18 columns with 4.5% ACN in 0.1% FA isocratic for 30 min. Fractions were subject to MS characterization, where a dominating peak with m/z 453.75 (+2) was observed. Extracted ions chromatograms show this peak to be present in all active fractions. De novo sequencing of 453.75 peak gives [A]SDKPY[I,L][I,L]. N-terminal A is calculated from parent ion −A7. I and L are not resolved by MS because of equal molecular weights. Search of protein sequences gives only ASDKPYIL as match. ASDKPYIL is only found in alpha-actinin-2, a major muscle protein.

Stability of Peptides Ex Vivo.

Peptides are degraded by proteases in the gastrointestinal tract. However the speed of this degradation depends on the sequence of the peptide. In order to measure stability of the ASDKPYIL peptide series and to compare with e.g. RRKPYIL, 10 or 50 mg (wet weight) of mouse or rat intestinal tissue (distal ileum) was equilibrated in V-bottom 24 well plates in 800 μl HBSS, 25 mM HEPES, pH 7.4 at 37° C. with shaking at 350 rpm. Identical amounts of different peptides (final concentration of 1 μg/ml) were added to the intestinal pieces and incubation continued. At various time points, 100 μl aliquots were removed and diluted into whey protein hydrolysate (final concentration of 10 mg/ml) to non-specifically compete protease activity. Peptide solutions were then diluted and tested for bioactivity (FIG. 8). Peptides incubated under same conditions but in absence of intestine served as controls (no degradation). Determination of EC50 for stimulation of cells allowed calculation of recovered peptide (FIG. 9), assuming simple inactivation by the tissue.

### Example 2

1) Structure-activity relationship and stability (SAR)
a. Extended versions
b. Substituted versions

2) In vivo studies in mice
c. Acute effects on feed intake (satiety)
d. Long-term effects on weight may be determined

Based on structural modelling studies of DC7-2 and NTR-1 interactions, peptides being octapeptides, heptapeptides, hexapeptides, or pentapeptides to exhibit increased potency due to increased binding may be predicted.

Comparison with SAR studies using synthetic peptides, peptides with increased potency and stability may be both predicted and observed.

Assays:

Synthetic Peptides:

Based on the sequence of the natural hormone Neurotensin (QLYENKPRRPYIL), the bioactive Neurotensin fragment NT(8-13)(RRPYIL) and the identified bioactive octapeptide DC7-2 (ASDKPYIL), synthetic peptides with systematic substitutions of N-terminal amino acids of the octapeptide (X-SDKPYIL), the heptapeptide (X-DKPYIL), the hexapeptide (X-KPYIL) and the pentapeptide (X-PYIL) were synthesised using standard techniques (Schafer-N, Denmark). All peptides were dissolved in pure HPLC-grade water and stored at −20° C.

Stability of Peptides:

Concentration Determination:

Protein concentration of synthetic peptides (Schafer-N, Denmark), NT (Sigma-Aldrich, Denmark) and NT (8-13)(Sigma-Aldrich, Denmark) were determined by measuring absorbance at 280 nm in Costar® 96-well UV-transparent plates (Corning, Sigma-Aldrich, Denmark). Each peptide was measured in 4 different concentrations by dilution in Hank's balanced salt solution (HBSS, ThermoFischer Scientific, Denmark) containing 25 mM HEPES (pH 7.4) (Sigma-Aldrich, Denmark). For stability assays, all peptides were diluted to 3×10⁻⁵ M in HBSS; 25 mM HEPES (Ph 7.4) and stored at +4° C.

Intestine Homogenate:

Small intestines from 20 Swiss-Webster males were homogenized in 350 ml Dulbecco's phosphate-buffered saline (PBS) (pH 7.4) (ThermoFischer Scientific, Denmark) with a IKA® basic 18 Ultra-Turrax tissue homogenizer set a speed 5 followed by filtration using 100 μm nylon mesh filter. Protein concentration was 6 mg/ml using the bicinconinic acid assay (ThermoFischer Scientific, Denmark) and bovine serum albumin as standard. The intestine homogenate was diluted 10 times in HBSS containing 25 mM HEPES (pH 7.4), and further diluted 30×, 90×, 270×, 810× or 2430×before incubation with peptides. All solutions were prewarmed to 37° C. before mixing with peptide solutions.

Peptides were incubated at 10⁻⁵ M with dilutions of small intestine homogenate at 37° C. for 90 minutes with shaking. Reactions were stopped by addition of 1 M phosphoric acid (final 0.4 M, pH ˜1.2). Each peptide incubation mix was then neutralized with NaOH to pH 7.2-7.4 and immediately tested for activity in intestinal cells. Control for zero degradation, i.e. addition of 1 M phosphoric acid prior to addition of intestine homogenate, was included for each peptide.

Fetal Bovine Serum:

All peptides were incubated at 10⁻⁵ M with Fetal Bovine Serum (FBS; final concentration of 66.7%) (Sigma Aldrich, Denmark) at 37° C. for 3 hours. The peptide degradation was terminated using 1 M phosphoric acid (final 0.4 M, pH ˜1.2) and neutralized to pH 7.2-7.4 with NaOH before testing activity in intestinal cells. As for small intestine homogenates, a zero degradation control was included for each peptide.

Kinetic Studies of Selected Peptides:

DC7-2, NT, DKPYIL and NT-(8-13) (final concentration of 10⁻⁶ M) were incubated either with FBS or with 270× diluted intestinal homogenate at 37° C. for various time points with shaking. Degradation was stopped with 1 M phosphoric acid and the samples were subsequently neutralized and immediately tested with intestinal cells as described above. Control for zero degradation was included for each peptide as above.

Study of Hexapeptides:

The 20 hexapeptides with systematic N-terminal substitutions (X-KPYIL) (Schafer-N, Denmark) and NT (8-13) was incubated at 10⁻⁶ M in either FBS for 10 minutes or with 270× diluted intestinal homogenate for 30 minutes at 37° C. with shaking. The degradation was stopped with 1 M phosphoric acid. Peptide solutions were neutralized with NaOH (pH 7.2-7.4), diluted and immediately tested for bioactivity using murine intestinal cells. Determination of EC50 for stimulation of cells allowed calculation of recovered peptide. Systematic substitutions of N-terminal amino acids in octapeptide ASDKPYIL and their importance for activity and stability. Sequence, activity and stability of DC7-2 is italicized.

| | | | | | Stability in |
|---|---|---|---|---|---|
| | | Cell signaling activity | | Stability in serum | intestine |
| | | (EC50, nM) | | (activity | (activity |
| Peptide ID | Sequence | Mean | SEM | remaining)¹⁾ | remaining)²⁾ |
| 36055 | Y SDKPYIL (SEQ ID NO: 939) | 5.92E−09 | 5.53E−10 | 0.0491 | 3.0 |
| 36054 | W SDKPYIL (SEQ ID NO: 940) | 8.87E−09 | 7.94E−10 | 0.0426 | 3.6 |
| 36053 | V SDKPYIL (SEQ ID NO: 795) | 5.15E−09 | 5.32E−10 | 0.0488 | 5.6 |
| 36052 | T SDKPYIL (SEQ ID NO: 941) | 5.63E−09 | 5.67E−10 | 0.0535 | 8.4 |
| 36051 | S SDKPYIL (SEQ ID NO: 942) | 4.27E−09 | 3.92E−10 | 0.0609 | 19.5 |
| 36050 | R SDKPYIL (SEQ ID NO: 943) | 4.01E−09 | 4.43E−10 | 0.0735 | 2.6 |
| 36049 | Q SDKPYIL (SEQ ID NO: 944) | 4.01E−09 | 4.51E−10 | 0.0742 | 8.8 |
| 36048 | P SDKPYIL (SEQ ID NO: 945) | 4.14E−09 | 4.54E−10 | 0.0772 | 4.6 |
| 36047 | N SDKPYIL (SEQ ID NO: 946) | 4.41E−09 | 4.28E−10 | 0.0872 | 3.9 |
| 36046 | M SDKPYIL (SEQ ID NO: 947) | 4.88E−09 | 5.01E−10 | 0.0762 | 3.8 |
| 36045 | L SDKPYIL (SEQ ID NO: 507) | 8.06E−09 | 8.25E−10 | 0.0917 | 7.9 |
| 36044 | K SDKPYIL (SEQ ID NO: 948) | 1.07E−08 | 1.05E−09 | 0.0527 | 6.6 |
| 36043 | I SDKPYIL (SEQ ID NO: 651) | 6.87E−09 | 7.41E−10 | 0.0956 | 6.0 |
| 36042 | H SDKPYIL (SEQ ID NO: 949) | 3.63E−09 | 3.24E−10 | 0.0980 | 6.4 |
| 36041 | G SDKPYIL (SEQ ID NO: 950) | 4.07E−09 | 5.07E−10 | 0.0912 | 10.3 |
| 36040 | F SDKPYIL (SEQ ID NO: 951) | 3.85E−09 | 4.95E−10 | 0.0944 | 3.4 |
| 36039 | E SDKPYIL (SEQ ID NO: 952) | 4.82E−09 | 5.76E−10 | 0.0964 | 23.0 |
| 36038 | D SDKPYIL (SEQ ID NO: 953) | 6.15E−09 | 6.85E−10 | 0.0947 | 29.9 |
| 36037 | C SDKPYIL (SEQ ID NO: 954) | 5.44E−09 | 6.25E−10 | 0.0963 | 14.0 |
| 36036 | A SDKPYIL (SEQ ID NO: 6) | 3.51E−09 | 4.75E−10 | 0.0988 | 6.0 |

Notes for Tables
¹⁾Stability in serum is expressed as fraction of peptide activity left after 10 min of incubation in serum at 37° C. compared with undigested sample as described in Examples.
²⁾Stability in intestine is expressed as % activity left after 30 min incubation in intestine homogenate at 37° C. as described in Examples.

Systematic substitutions of N-terminal amino acid in heptapeptide SDKPYIL and their importance for activity and stability. Sequence, activity and stability of peptide contained in DC7-2 is italicized.

| | | Cell signaling activity | | Stability in | Stability in |
|---|---|---|---|---|---|
| | | (EC50, nM) | | serum | intestine |
| Peptide ID | Sequence | Mean | STD | (t½, min) | (t½, min) |
| 36035 | Y DKPYIL (SEQ ID NO: 955) | 6.83E−09 | 5.97E−10 | 0.0531 | 3.2 |
| 36034 | W DKPYIL (SEQ ID NO: 956) | 1.41E−08 | 1.18E−09 | 0.0427 | 6.3 |
| 36033 | V DKPYIL (SEQ ID NO: 957) | 4.94E−09 | 4.75E−10 | 0.0528 | 4.4 |
| 36032 | T DKPYIL (SEQ ID NO: 292) | 5.61E−09 | 5.33E−10 | 0.0593 | 13.3 |
| 36031 | S DKPYIL (SEQ ID NO: 7) | 5.00E−09 | 4.88E−10 | 0.0587 | 10.6 |
| 36030 | R DKPYIL (SEQ ID NO: 958) | 4.68E−09 | 4.77E−10 | 0.0597 | 6.9 |
| 36029 | Q DKPYIL (SEQ ID NO: 959) | 4.97E−09 | 4.86E−10 | 0.0620 | 11.5 |
| 36028 | P DKPYIL (SEQ ID NO: 960) | 4.67E−09 | 4.64E−10 | 0.0558 | 10.9 |
| 36027 | N DKPYIL (SEQ ID NO: 961) | 5.92E−09 | 5.21E−10 | 0.0580 | 40.5 |
| 36026 | M DKPYIL (SEQ ID NO: 962) | 6.08E−09 | 5.69E−10 | 0.0601 | 7.1 |
| 36025 | L DKPYIL (SEQ ID NO: 963) | 6.41E−09 | 9.98E−10 | 0.0969 | 3.9 |
| 36024 | K DKPYIL (SEQ ID NO: 964) | 1.12E−08 | 1.61E−09 | 0.0910 | 6.9 |
| 36023 | I DKPYIL (SEQ ID NO: 965) | 4.77E−09 | 8.27E−10 | 0.0928 | 2.8 |
| 36022 | H DKPYIL (SEQ ID NO: 966) | 2.65E−09 | 3.53E−10 | 0.0932 | 4.7 |
| 36021 | G DKPYIL (SEQ ID NO: 967) | 2.91E−09 | 3.86E−10 | 0.0920 | 4.5 |
| 36020 | F DKPYIL (SEQ ID NO: 968) | 9.52E−09 | 1.38E−09 | 0.0901 | 2.4 |
| 36019 | E DKPYIL (SEQ ID NO: 969) | 3.96E−09 | 6.89E−10 | 0.0846 | 17.2 |
| 36018 | D DKPYIL (SEQ ID NO: 970) | 1.05E−08 | 1.47E−09 | 0.0419 | 44.3 |
| 36017 | C DKPYIL (SEQ ID NO: 971) | 7.72E−09 | 1.17E−09 | 0.0407 | 9.4 |
| 36016 | A DKPYIL (SEQ ID NO: 972) | 3.52E−09 | 6.43E−10 | 0.0952 | 2.5 |

Systematic substitutions of N-terminal amino acid in hexapeptide DKPYIL and their importance for activity and stability. Sequence, activity and stability of peptide contained in DC7-2 is italicized.

| | | Cell signaling activity | | Stability in | Stability in |
|---|---|---|---|---|---|
| | | (EC50, nM) | | serum (t½, | intestine |
| Peptide ID | Sequence | Mean | STD | min) | (t½, min) |
| 35995 | Y KPYIL (SEQ ID NO: 973) | 3.34E−09 | 5.33E−10 | 0.0346 | 0.1 |
| 35994 | W KPYIL (SEQ ID NO: 974) | 5.58E−09 | 8.57E−10 | 0.0334 | 0.2 |
| 35993 | V KPYIL (SEQ ID NO: 975) | 1.17E−09 | 1.73E−10 | 0.0375 | 0.2 |
| 35992 | T KPYIL (SEQ ID NO: 976) | 1.16E−09 | 1.72E−10 | 0.0424 | 0.3 |
| 35991 | S KPYIL (SEQ ID NO: 977) | 1.15E−09 | 1.70E−10 | 0.0477 | 1.3 |
| 35990 | R KPYIL (SEQ ID NO: 150) | 4.40E−10 | 6.59E−11 | 0.0480 | 0.1 |
| 35989 | Q KPYIL (SEQ ID NO: 978) | 3.78E−10 | 5.67E−11 | 0.0495 | 0.8 |
| 35988 | P KPYIL (SEQ ID NO: 979) | 2.32E−10 | 3.53E−11 | 0.0550 | 0.2 |
| 35987 | N KPYIL (SEQ ID NO: 980) | 5.00E−10 | 7.42E−11 | 0.0709 | 1.0 |
| 35986 | M KPYIL (SEQ ID NO: 981) | 3.88E−10 | 5.82E−11 | 0.0504 | 0.1 |
| 35985 | L KPYIL (SEQ ID NO: 982) | 3.30E−10 | 4.60E−11 | 0.0310 | 0.1 |
| 35984 | K KPYIL (SEQ ID NO: 43) | 2.64E−10 | 3.72E−11 | 0.0403 | 0.1 |
| 35983 | I KPYIL (SEQ ID NO: 983) | 2.40E−10 | 3.37E−11 | 0.0315 | 0.0 |
| 35982 | H KPYIL (SEQ ID NO: 984) | 2.71E−10 | 3.82E−11 | 0.0363 | 0.1 |
| 35981 | G KPYIL (SEQ ID NO: 184) | 3.64E−10 | 5.05E−11 | 0.0353 | 0.1 |
| 35980 | F KPYIL (SEQ ID NO: 985) | 3.15E−10 | 4.39E−11 | 0.0365 | 0.1 |
| 35979 | E KPYIL (SEQ ID NO: 114) | 5.65E−10 | 7.80E−11 | 0.0478 | 0.6 |
| 35978 | D KPYIL (SEQ ID NO: 8) | 8.03E−10 | 1.11E−10 | 0.0623 | 2.2 |
| 35977 | C KPYIL (SEQ ID NO: 986) | 1.11E−09 | 1.53E−10 | 0.0477 | 2.6 |
| 35976 | A KPYIL (SEQ ID NO: 987) | 2.51E−10 | 3.50E−11 | 0.0454 | 0.1 |

Systematic substitutions of N-terminal amino acid in pentapeptide KPYIL and their importance for activity and stability. Sequence, activity and stability of peptide contained in DC7-2 is italicized.

| | | Cell signaling activity | | Stability in | Stability in |
|---|---|---|---|---|---|
| | | (EC50, nM) | | serum (t½, | intestine (t½, |
| Peptide ID | Sequence | Mean | STD | min) | min) |
| 36015 | Y PYIL (SEQ ID NO: 988) | 1.40E−07 | 1.01E−08 | 0.0091 | 2.0 |
| 36014 | W PYIL (SEQ ID NO: 989) | 1.33E−07 | 1.32E−08 | 0.0066 | 0.2 |
| 36013 | V PYIL (SEQ ID NO: 990) | 3.78E−08 | 2.71E−09 | 0.0094 | 0.3 |
| 36012 | T PYIL (SEQ ID NO: 991) | 4.36E−08 | 3.13E−09 | 0.0163 | 2.9 |
| 36011 | S PYIL (SEQ ID NO: 992) | 2.25E−08 | 1.62E−09 | 0.0241 | 0.0 |
| 36010 | R PYIL (SEQ ID NO: 40) | 1.18E−09 | 1.00E−10 | 0.0176 | 1.1 |
| 36009 | Q PYIL (SEQ ID NO: 993) | 2.05E−08 | 1.47E−09 | 0.0372 | 0.2 |
| 36008 | P PYIL (SEQ ID NO: 994) | 9.61E−09 | 6.98E−10 | 0.0197 | 2.6 |
| 36007 | N PYIL (SEQ ID NO: 995) | 3.93E−08 | 2.81E−09 | 0.0148 | 0.4 |
| 36006 | M PYIL (SEQ ID NO: 996) | 1.62E−08 | 1.17E−09 | 0.0034 | 0.2 |
| 36005 | L PYIL (SEQ ID NO: 997) | 4.10E−08 | 4.52E−09 | 0.0373 | 0.1 |
| 36004 | K PYIL (SEQ ID NO: 9) | 7.71E−09 | 8.77E−10 | 0.0124 | 0.2 |
| 36003 | I PYIL (SEQ ID NO: 998) | 2.38E−08 | 2.63E−09 | 0.0132 | 0.2 |
| 36002 | H PYIL (SEQ ID NO: 999) | 3.56E−08 | 3.94E−09 | 0.0366 | 0.1 |
| 36001 | G PYIL (SEQ ID NO: 1000) | 1.74E−08 | 1.94E−09 | 0.0125 | 0.3 |
| 36000 | F PYIL (SEQ ID NO: 1001) | 1.95E−08 | 2.17E−09 | 0.0343 | 2.4 |
| 35999 | E PYIL (SEQ ID NO: 1002) | 1.02E−07 | 1.12E−08 | 0.0405 | 3.6 |
| 35998 | D PYIL (SEQ ID NO: 1003) | 1.58E−07 | 1.74E−08 | 0.0352 | 12.5 |
| 35997 | C PYIL (SEQ ID NO: 1004) | 6.99E−08 | 7.73E−09 | 0.0349 | 0.2 |
| 35996 | A PYIL (SEQ ID NO: 1005) | 1.18E−08 | 1.31E−09 | 0.0036 | 0.4 |

In conclusion, the results demonstrates that octa- and heptapeptides are more stable, and that the N-terminal aa in the hexapeptide has a significant implication on the stability.

As compared to the hexapeptide of a natural homone, neurotensin (8-13) (NT with the sequence RRPYIL), one specific peptide of the present invention DKPYIL is nearly 100 times more stable in serum and around 100-1000× more stable in intestine homogenate.

### Example 3

Synergistic Effect of Dietary Peptides and GLP-1 Analogue on Gastric Emptying.

Both DC7-2 (0.43 mg/kg; 4.3 mg/kg; 43 mg/kg; 128 mg/kg i.p.) and liraglutide (0.023 mg/kg; 0.22 mg/kg; 3.23 mg/kg; 8.66 mg/kg; 35 mg/kg s.c.) potently inhibited gastric emptying rates in a dose-dependent manner, as shown in FIGS. 17A and C, with an EC₅₀ value of 3 mg/kg and 60 μg/kg, respectively. Liraglutide reached its maximum inhibition of gastric emptying at doses higher than 3 mg/kg retaining approximately 30% of the phenol red marker solution in the stomach. In contrast, DC7-2 was able to inhibit gastric emptying more extensively than liraglutide showing almost a 50% inhibition using doses at 128 mg/kg or higher. A combination test was designed in which DC7-2 was given at EC₅₀ (3 mg/kg) together with different doses of liraglutide (same as used in dose-response test) (FIG. 17 B). Clearly, the combination of DC7-2 with liraglutide was 2-fold more potent than liraglutide alone with an EC₅₀ value of 30 μg/kg. Furthermore, the DC7-2+liraglutide combination was also more efficient in delaying gastric emptying showing an almost 70% inhibition, i.e. more than a 2-fold improvement compared to liraglutide alone and a 1.5-fold improvement compared to maximum of DC7-2 alone. To display if the effect of liraglutide in combination with DC7-2 was additive or synergistic, we calculated and plotted the sum of liraglutide alone and DC7-2 (3 mg/kg) alone shown as the grey curve in FIG. 17 B. Since the combination data from the experiment (black curve) was above the grey curve at all data points, the combination of liraglutide and DC7-2 clearly suggested a synergistic mode-of-action in inhibiting gastric emptying indicating the use of two different pathways. This was further supported by the different maximum values observed for liraglutide and DC7-2 alone, respectively.

Chronic and Synergistic Effect of DC7-2, DPPIV Inhibitor and GLP-1 Analogue on Feed Intake.

The effect of DC7-2 alone or in combination with liraglutide on feed intake and body weight was investigated in outbred Swiss-Webster females. For liraglutide, a dose of 100 μg/kg (0.003 mg per mouse) was used as this has been shown to be effective for food intake in mice and close to our EC50 value in gastric emptying determination. On the basis of the acute feed intake data, 30 mg/kg corresponding to 1 mg/mouse dose of DC7-2 was chosen as it led to a marked reduction in feed intake.

Both DC7-2 alone and liraglutide alone significantly reduced feed intake after seven days of daily dosing in comparison to vehicle. However, the combination of liraglutide and DC7-2 was not different from liraglutide or DC7-2 alone (FIG. 18). Whereas the effects of liraglutide on feed intake lasted throughout the experiment (3 weeks), the effects of DC7-2 almost vanished after the first week, and no effect on feed intake was evident after week 1. Actually, DC7-2 treated mice had an increased feed intake compared to vehicle in week 3 indicating that mice were over-compensating which might be explained by a desensitization effect when the NT receptors are exposed to high concentrations of agonist of a longer time period. The combinational treatment of liraglutide with DC7-2 showed reduced feed intake to the same extent as liraglutide alone, suggesting that liraglutide may suppress the over-compensating effect on feed intake observed in the DC7-2 group or that the dose of liraglutide was simply too high to observe any effect of DC7-2 in this setup. In agreement with the observations in chronic feed intake, a decrease in body weight was observed for the liraglutide-treated groups only, while no effect was observed for the DC7-2-treated group.

The mechanism behind the anorexigenic effect of DC7-2 might be more than only inhibition of gastric emptying. The co-expression and co-secretion with other anorectic hormones or metabolites, a possible synergistic effect with other gut hormones, or a possible effect on glucose homeostasis might also enhance the appetite control.

Although the appetite regulating receptors work in different pathways: NT receptor is coupled to G_{q/11}, PYY receptor is coupled to Gᵢ and GLP-1 receptor to Gₛ, NT was reported to co-express and co-release with PYY and GLP-1 in the GI tract. Our result showed the synergistic effect of the GLP-1 analogue, liraglutide, and DC7-2 (FIG. 17), suggesting the co-action of NT-like peptides with other hormones in vivo. Thus, our hypothesis was that DC7-2 reduced feed intake due to the delayed gastric emptying, directly mediated by NTR, and at the same time increased the secretion of several anorexigenic hormones such as NT, CCK, GLP-1 and PYY, which also contributed to a further delay of gastric emptying and finally, reduced feed intake.

The chronic test of DC7-2 and liraglutide on feed intake suggested a long-term anorexigenic effect in vivo as well as an effect of desensitization (FIG. 18). G protein systems were shown to exhibit memories from previous activations, therefore previous exposure to low concentration of activators induce high activation (sensitization) whereas by subsequent exposure to high concentration of activators lead to lower sensitivity and signalling (desensitization). In details, GPCRs become phosphorylated by GPCR kinases (GRKs) and then bound to arrestins to inhibit further stimulation from G proteins. For example, preincubation of exendin-4 (a GLP-1 analogue) for 5-120 min (acute) or 24-72 hours (chronic) both significantly reduced the sensitivity of GLP-1R in INS-1 cells, however, 1 week of daily exposure with exendin-4 in mice showed a tendency but not significant difference in desensitization of glucose homeostasis. In our test, anorexigenic effects of DC7-2 and GLP-1 were reduced after 3-week administration, which requires further investigation.

In summary, we established both the acute and chronic anorexigenic effect of DC7-2 in vivo, and revealed that the mechanism at least partially is mediated by delayed gastric emptying.

### Example 4

DC7-2 Reduces Blood Sugar Excursion after an Oral Glucose Tolerance Test in Mice

We hypothesized that the effect of DC7-2 in delaying gastric emptying would lead to a slower appearance of glucose in the blood following an oral load. This would be a desirable effect in Type 2 diabetes.

Surprisingly, the blood glucose peak and AUC were both significantly lower following administration of DC7-2. Blood glucose immediately prior to oral glucose challenge was also significantly reduced by DC7-2.

Subsequently, we decided to fast mice o/n instead of only 4 h—this caused the basal blood glucose to drop from ˜7 mM (FIG. 19) to ˜3 mM (FIG. 20), and we prepared plasma from the blood samples in order to also determine insulin levels. In addition, we decided to include Liraglutide (4 ug per mouse) administered s.c. at t=−40 min, either alone or in combination with DC7-2 as was previously done for measuring gastric emptying.

As expected, Liraglutide reduced the blood sugar excursion, but the combination of DC7-2 and Liraglutide showed an even better blood sugar profile with additive effects of DC7-2 and the GLP-1 analog. DC7-2 clearly delays glucose appearance in the plasma and reduces peak levels comparable to that observed for Liraglutide alone, but peak level of plasma glucose for the combination treatment is even more dramatically reduced and reaches only half of that obtained for the control.

Plasma samples from four independent experiments each with four mice per group and per time point were then pooled and used for determination of both glucose and insulin in the same samples. This gives n=16 mice per group and per time point.

Insulin levels are increased for Liraglutide as expected, but DC7-2 does not appear to increase insulin levels (no significant effect on peak level or AUC). Rather, DC7-2 causes a delay in insulin response and follows appearance of blood glucose as in FIG. 20. The same is observed for the combination of Liraglutide and DC7-2.

We next did a dose-response, combining results from the fixed dose used for FIGS. 20 and 21 with a single experiment with varying doses of either Liraglutide or DC7-2 (FIG. 22).

### Example 5

Lean-control and Diet-Induced Obese mice (C57Bl/6J) were randomly assigned into four groups after 5-hour fasting. Groups were subcutaneously treated with liraglutide or vehicle (controls and DC7-2 group) at −40 min. Mice were then treated intraperitoneally with DC7-2 or vehicle at time 0 and immediately gavaged with 20% glucose solution. Plasma glucose was determined at the indicated time points for up to 180 min. Results are mean+/−SEM (n=8 mice per group). Areas under the curve (AUC) were calculated for each group. AUC with different superscripts are significantly different (P<0.01).

1. A combination of
i) a first polypeptide comprising the amino acid sequence
(formula I; SEQ ID NO: 1)
AA1-AA2-AA3-K-AA5-AA6-AA7-AA8
or
AA2-AA3-K-AA5-AA6-AA7-AA8
or
AA3-K-AA5-AA6-AA7-AA8
or
K-AA5-AA6-AA7-AA8,
wherein AA1 when present is an amino acid selected from A, L, I, and V; AA2 when present is an amino acid selected from S, T, G, A, N, E and D; AA3 when present is an amino acid selected from D, E, and G; AA5 is selected from P, N, S, D, A, T, K, and G; AA6 is selected from Y, N, I, W, and F; AA7 is selected from I, L, R, and V; AA8 is selected from L, I, V, S, M, and T; which polypeptide is not more than 50 amino acids in length; and
ii) a second peptide hormone, which is a gut hormone.

2.-4. (canceled)

5. The combination according to claim 1, wherein said first polypeptide is extended from A.A8 in the C-terminal by 2-10 additional amino acids and/or wherein said first polypeptide has its N-terminal amino acid selected from AA1, AA2, AA3, or the K in position 4 of SEQ ID NO:1.

6. (canceled)

7. The combination according claim 1, wherein said first polypeptide is a polypeptide consisting of the amino acid sequence
(formula II, SEQ ID NO: 2)
R1-AA1-AA2-AA3-K-AA5-AA6-AA7-AA8-R2,
wherein R1 defines the N-term (—NH2) or a protection group; and R2 defines the C-term (—COOH).

8.-13. (canceled)

14. The combination according to claim 1, wherein said first polypeptide is a polypeptide, wherein AA1 is A and/or AA2 is S and/or AA3 is D and/or AA5 is P and/or AA6 is Y and/or AA7 is I and/or AA8 is L.

15.-22. (canceled)

23. The combination according to claim 1, wherein said first polypeptide is a polypeptide, which amino acid sequence only contains naturally occurring amino acids.

24. The combination according to claim 1, wherein said first polypeptide is a polypeptide, which is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

25.-26. (canceled)

27. The combination according to claim 1, wherein said first polypeptide is a polypeptide having or comprising a sequence selected from ASDKPYIL, AGDKNYIL, AGDKNYIT, AGDKSYIT, ADGKPYIV, AEDKDFIT, AADKPYIL, ATDKPYIL, AGDKPYIT, ASEKPYIL, ADGKPYVT, AG DKPYIL, ASDKPNIL, ASDKPYIT, AADKPFIL, ASDKAYIT, AGDKAYIT, ANGKPFIT, AGDKNFIT, ASDKSYIT, ASDKTYIT, ASDKNYIT, AGDKKYIT, AGDKNYIS, AADKNYIT, AGDKNYIM, AADKNFIM, AADKNFIT, AGDKGIRS, DKPYIL, and KPYIL.

28.-31. (canceled)

32. The combination according to claim 1, wherein said first polypeptide has been modified by N terminal acylation or other protection groups.

33. The combination according to claim 1, wherein said second peptide hormone is selected from the group consisting of Cholecystokinin (CCK), Gastrin, Secretin, Vasoactive Intestinal Peptide (VIP), Glucose-dependent insulinotropic peptide (GIP), Glucagon-like Peptide 1 and 2 (GLP-1 and -2), Bombesin, Chromogranin A, Glucagon, Insulin, Leptin, Neuropeptide Y, Neurotensin, Neuromedin, Pancreatic Polypeptide, PYY, Amylin, Oxyntomodulin, Xexin, Motilin, Grehlin, and Somatostatin, and bioactive analogues or variants of any one of these peptide hormones, such as a polypeptide being a GLP-1 receptor agonist is selected from the group consisting of: GLP-1(7-37); GLP-1(7-36) amide; Oxyntomodulin; Exenatide; Exenatide LAR; Liraglutide; Semaglutide; Taspoglutide; Albiglutide; Lixisenatide; and Dulaglutide.

34. A composition comprising a combination according to claim 1, wherein the composition is an oral dosage form.

35.-37. (canceled)

38. The composition according to claim 34, wherein the energy content derived through the process of cellular respiration is less than 50 kilojoules (kJ), less than 40 kJ, less than 30 kJ, less than 20 kJ, less than 10 kJ, less than 5000 Joules (J), less than 1000 J, less than 900 J, less than 800 J, less than 700 J, less than 600 J, less than 500 J, less than 400 J, less than 300 J, less than 200 J, less than 100 J, or less than 50 J.

39. The composition according to claim 34, wherein the composition is
a food composition, or
a fermented composition, or
a pharmaceutical composition, or
a nutritional composition.

40.-44. (canceled)

45. The composition according to claim 34, which is an oral dosage form selected from the group consisting of tablets, capsules, caplets, slurries, sachets, suspensions, chewing gum, and powder formulation that may be dissolved in a liquid, comprising water, milk, juice, or yogurt.

46.-51. (canceled)

52. A method of promoting satiety or
for reducing feed intake or
for preventing or reducing the incidence of obesity or
for reducing or treating cardiovascular diseases, atherosclerosis, hypertension, hepatosteatosis, cancer and/or diabetes
in a subject, comprising enteral administering to a subject in need thereof a combination of
i) a first polypeptide comprising or consisting of the amino acid sequence
(formula III, SEQ ID NO: 3)
AA1-AA2-AA3-AA4-AA5-AA6-AA7-AA8,
wherein AA1 when present is an amino acid selected from A, L, I, and V; AA2 when present is an amino acid selected from S, T, G, A, N, E and D; AA3 when present is an amino acid selected from D, R, K, E, and G; AA4 is an amino acid selected from K and R; AA5 is selected from P, N, S, D, A, T, and G; AA6 is selected from Y, N, I, W, and F; AA7 is selected from I, L, R, and V; AA8 is selected from L, I, V, S, M, and T; which polypeptide is not more than 50 amino acids in length; or a variant thereof with a sequence identity of at least 80%; and
ii) a second peptide hormone, such as a polypeptide being a GLP-1 receptor agonist.

53.-62. (canceled)


SEQUENCE LISTING (1005 sequences)

SEQ ID NO: 1 - protein, 8 aa, Artificial Sequence
  Artificial peptide sequence
  MISC_FEATURE (1)..(1): Xaa in position 1 is A, L, I, or V
  MISC_FEATURE (2)..(2): Xaa in position 2 is S, T, G, A, N, E or D
  MISC_FEATURE (3)..(3): Xaa in position 3 is D, E, or G
  MISC_FEATURE (5)..(5): Xaa in position 5 is P, N, S, D, A, T, K, or G
  MISC_FEATURE (6)..(6): Xaa in position 6 is Y, N, I, W, or F
  MISC_FEATURE (7)..(7): Xaa in position 7 is I, L, R, or V
  MISC_FEATURE (8)..(8): Xaa in position 8 is L, I, V, S, M, or T
XXXKXXXX

SEQ ID NO: 2 - protein, 8 aa, Artificial Sequence
  Artificial peptide sequence
  MISC_FEATURE (1)..(1): Xaa in position 1 is N-term (-NH2) A, L, I, or V
  MISC_FEATURE (2)..(2): Xaa in position 2 is S, T, G, A, N, E or D
  MISC_FEATURE (3)..(3): Xaa in position 3 is D, E, or G
  MISC_FEATURE (5)..(5): Xaa in position 5 is P, N, S, D, A, T, K, or G
  MISC_FEATURE (6)..(6): Xaa in position 6 is Y, N, I, W, or F
  MISC_FEATURE (7)..(7): Xaa in position 7 is I, L, R, or V
  MISC_FEATURE (8)..(8): Xaa in position 8 is a C-term L, I, V, S, M, or T
XXXKXXXX

SEQ ID NO: 3 - protein, 8 aa, Artificial Sequence
  Artificial peptide sequence
  MISC_FEATURE (1)..(1): Xaa in position 1 is A, L, I, or V
  MISC_FEATURE (2)..(2): Xaa in position 2 is S, T, G, A, N, E or D
  MISC_FEATURE (3)..(3): Xaa in position 3 is D, R, K, E, or G
  MISC_FEATURE (4)..(4): Xaa in position 4 is K or R
  MISC_FEATURE (5)..(5): Xaa in position 5 is P, N, S, D, A, T, K, or G
  MISC_FEATURE (6)..(6): Xaa in position 6 is Y, N, I, W, or F
  MISC_FEATURE (7)..(7): Xaa in position 7 is I, L, R, or V
  MISC_FEATURE (8)..(8): Xaa in position 8 is L, I, V, S, M, or T
XXXXXXXX

SEQ ID NO: 4 - protein, 4 aa, Artificial Sequence
  Artificial peptide sequence
PYIL

SEQ ID NO: 5 - protein, 16 aa, Artificial Sequence
  Artificial peptide sequence
AVTEKKYILYDFSVTS

SEQ ID NO: 6 - protein, 8 aa, Artificial Sequence
  Artificial peptide sequence
ASDKPYIL

SEQ ID NO: 7 - protein, 7 aa, Artificial Sequence
  Artificial peptide sequence
SDKPYIL

SEQ ID NO: 8 - protein, 6 aa, Artificial Sequence
  Artificial peptide sequence
DKPYIL

SEQ ID NO: 9 - protein, 5 aa, Artificial Sequence
  Artificial peptide sequence
KPYIL

SEQ ID NO: 10 - protein, 8 aa, Artificial Sequence
  Artificial peptide sequence
AGDKNYIL

SEQ ID NO: 11 - protein, 8 aa, Artificial Sequence
  Artificial peptide sequence
AGDKNYIT

SEQ ID NO: 12 - protein, 8 aa, Artificial Sequence
  Artificial peptide sequence
AGDKSYIT

SEQ ID NO: 13 - protein, 8 aa, Artificial Sequence
  Artificial peptide sequence
ADGKPYIV

SEQ ID NO: 14 - protein, 8 aa, Artificial Sequence
  Artificial peptide sequence
AEDKDFIT

SEQ ID NO: 15 - protein, 8 aa, Artificial Sequence
  Artificial peptide sequence
AADKPYIL

SEQ ID NO: 16 - protein, 8 aa, Artificial Sequence
  Artificial peptide sequence
ATDKPYIL

SEQ ID NO: 17 - protein, 8 aa, Artificial Sequence
  Artificial peptide sequence
AGDKPYIT

SEQ ID NO: 18 - protein, 8 aa, Artificial Sequence
  Artificial peptide sequence
ASEKPYIL

SEQ ID NO: 19 - protein, 8 aa, Artificial Sequence
  Artificial peptide sequence
ADGKPYVT

SEQ ID NO: 20 - protein, 8 aa, Artificial Sequence
  Artificial peptide sequence
AGDKPYIL

SEQ ID NO: 21 - protein, 8 aa, Artificial Sequence
  Artificial peptide sequence
ASDKPNIL

SEQ ID NO: 22 - protein, 8 aa, Artificial Sequence
  Artificial peptide sequence
ASDKPYIT

SEQ ID NO: 23 - protein, 8 aa, Artificial Sequence
  Artificial peptide sequence
AADKPFIL

SEQ ID NO: 24 - protein, 8 aa, Artificial Sequence
  Artificial peptide sequence
ASDKAYIT

SEQ ID NO: 25 - protein, 8 aa, Artificial Sequence
  Artificial peptide sequence
AGDKAYIT

SEQ ID NO: 26 - protein, 8 aa, Artificial Sequence
  Artificial peptide sequence
ANGKPFIT

SEQ ID NO: 27 - protein, 8 aa, Artificial Sequence
  Artificial peptide sequence
AGDKNFIT

SEQ ID NO: 28 - protein, 8 aa, Artificial Sequence
  Artificial peptide sequence
ASDKSYIT

SEQ ID NO: 29 - protein, 8 aa, Artificial Sequence
  Artificial peptide sequence
ASDKTYIT

SEQ ID NO: 30 - protein, 8 aa, Artificial Sequence
  Artificial peptide sequence
ASDKNYIT

SEQ ID NO: 31 - protein, 8 aa, Artificial Sequence
  Artificial peptide sequence
AGDKKYIT

SEQ ID NO: 32 - protein, 8 aa, Artificial Sequence
  Artificial peptide sequence
AGDKNYIS

SEQ ID NO: 33 - protein, 8 aa, Artificial Sequence
  Artificial peptide sequence
AADKNYIT

SEQ ID NO: 34 - protein, 8 aa, Artificial Sequence
  Artificial peptide sequence
AGDKNYIM

SEQ ID NO: 35 - protein, 8 aa, Artificial Sequence
  Artificial peptide sequence
AADKNFIM

SEQ ID NO: 36 - protein, 8 aa, Artificial Sequence
  Artificial peptide sequence
AADKNFIT

SEQ ID NO: 37 - protein, 8 aa, Artificial Sequence
  Artificial peptide sequence
AGDKGIRS

SEQ ID NO: 38 - protein, 7 aa, Artificial Sequence
  Artificial peptide sequence
PRRPYIL

SEQ ID NO: 39 - protein, 6 aa, Artificial Sequence
  Artificial peptide sequence
RRPYIL

SEQ ID NO: 40 - protein, 5 aa, Artificial Sequence
  Artificial peptide sequence
RPYIL

SEQ ID NO: 41 - protein, 6 aa, Artificial Sequence
  Artificial peptide sequence
RRPWIL

SEQ ID NO: 42 - protein, 6 aa, Artificial Sequence
  Artificial peptide sequence
KRPYIL

SEQ ID NO: 43 - protein, 6 aa, Artificial Sequence
  Artificial peptide sequence
KKPYIL

SEQ ID NO: 44 - protein, 6 aa, Artificial Sequence
  Artificial peptide sequence
  misc_feature (2)..(2): Xaa can be any naturally occurring amino acid
KXPYIL

SEQ ID NO: 45 - protein, 5 aa, Artificial Sequence
  Artificial peptide sequence
KPYII

SEQ ID NO: 46 - protein, 5 aa, Artificial Sequence
  Artificial peptide sequence
KPYIV

SEQ ID NO: 47 - protein, 5 aa, Artificial Sequence
  Artificial peptide sequence
KPYLL

SEQ ID NO: 48 - protein, 5 aa, Artificial Sequence
  Artificial peptide sequence
KPYLI

SEQ ID NO: 49 - protein, 5 aa, Artificial Sequence
  Artificial peptide sequence
KPYLV

SEQ ID NO: 50 - protein, 5 aa, Artificial Sequence
  Artificial peptide sequence
KPYVL

SEQ ID NO: 51 - protein, 5 aa, Artificial Sequence
  Artificial peptide sequence
KPYVI

SEQ ID NO: 52 - protein, 5 aa, Artificial Sequence
  Artificial peptide sequence
KPYVV

SEQ ID NO: 53 - protein, 5 aa, Artificial Sequence
  Artificial peptide sequence
KPWIL

SEQ ID NO: 54 - protein, 5 aa, Artificial Sequence
  Artificial peptide sequence
KPWII

SEQ ID NO: 55 - protein, 5 aa, Artificial Sequence
  Artificial peptide sequence
KPWIV

SEQ ID NO: 56 - protein, 5 aa, Artificial Sequence
  Artificial peptide sequence
KPWLL

SEQ ID NO: 57 - protein, 5 aa, Artificial Sequence
  Artificial peptide sequence
KPWLI

SEQ ID NO: 58 - protein, 5 aa, Artificial Sequence
  Artificial peptide sequence
KPWLV

SEQ ID NO: 59 - protein, 5 aa, Artificial Sequence
  Artificial peptide sequence
KPWVL

SEQ ID NO: 60 - protein, 5 aa, Artificial Sequence
  Artificial peptide sequence
KPWVI

SEQ ID NO: 61 - protein, 5 aa, Artificial Sequence
  Artificial peptide sequence
KPWVV

SEQ ID NO: 62 - protein, 5 aa, Artificial Sequence
  Artificial peptide sequence
RPYII

SEQ ID NO: 63 - protein, 5 aa, Artificial Sequence
  Artificial peptide sequence
RPYIV

SEQ ID NO: 64 - protein, 5 aa, Artificial Sequence
  Artificial peptide sequence
RPYLL

SEQ ID NO: 65 - protein, 5 aa, Artificial Sequence
  Artificial peptide sequence
RPYLI

SEQ ID NO: 66 - protein, 5 aa, Artificial Sequence
  Artificial peptide sequence
RPYLV

SEQ ID NO: 67 - protein, 5 aa, Artificial Sequence
  Artificial peptide sequence
RPYVL

SEQ ID NO: 68 - protein, 5 aa, Artificial Sequence
  Artificial peptide sequence
RPYVI

SEQ ID NO: 69 - protein, 5 aa, Artificial Sequence
  Artificial peptide sequence
RPYVV

SEQ ID NO: 70 - protein, 5 aa, Artificial Sequence
  Artificial peptide sequence
RPWIL

SEQ ID NO: 71 - protein, 5 aa, Artificial Sequence
  Artificial peptide sequence
RPWII

SEQ ID NO: 72 - protein, 5 aa, Artificial Sequence
  Artificial peptide sequence
RPWIV

SEQ ID NO: 73 - protein, 5 aa, Artificial Sequence
  Artificial peptide sequence
RPWLL

SEQ ID NO: 74 - protein, 5 aa, Artificial Sequence
  Artificial peptide sequence
RPWLI

SEQ ID NO: 75 - protein, 5 aa, Artificial Sequence
  Artificial peptide sequence
RPWLV

SEQ ID NO: 76 - protein, 5 aa, Artificial Sequence
  Artificial peptide sequence
RPWVL

SEQ ID NO: 77 - protein, 5 aa, Artificial Sequence
  Artificial peptide sequence
RPWVI

SEQ ID NO: 78 - protein, 5 aa, Artificial Sequence
  Artificial peptide sequence
RPWVV

SEQ ID NO: 79 - protein, 6 aa, Artificial Sequence
  Artificial peptide sequence
DKPYII

SEQ ID NO: 80 - protein, 6 aa, Artificial Sequence
  Artificial peptide sequence
DKPYIV

SEQ ID NO: 81 - protein, 6 aa, Artificial Sequence
  Artificial peptide sequence
DKPYLL

SEQ ID NO: 82 - protein, 6 aa, Artificial Sequence
  Artificial peptide sequence
DKPYLI

SEQ ID NO: 83 - protein, 6 aa, Artificial Sequence
  Artificial peptide sequence
DKPYLV

SEQ ID NO: 84 - protein, 6 aa, Artificial Sequence
  Artificial peptide sequence
DKPYVL

SEQ ID NO: 85 - protein, 6 aa, Artificial Sequence
  Artificial peptide sequence
DKPYVI

SEQ ID NO: 86 - protein, 6 aa, Artificial Sequence
  Artificial peptide sequence
DKPYVV

SEQ ID NO: 87 - protein, 6 aa, Artificial Sequence
  Artificial peptide sequence
DKPWIL

SEQ ID NO: 88 - protein, 6 aa, Artificial Sequence
  Artificial peptide sequence
DKPWII

SEQ ID NO: 89 - protein, 6 aa, Artificial Sequence
  Artificial peptide sequence
DKPWIV

SEQ ID NO: 90 - protein, 6 aa, Artificial Sequence
  Artificial peptide sequence
DKPWLL

SEQ ID NO: 91 - protein, 6 aa, Artificial Sequence
  Artificial peptide sequence
DKPWLI

SEQ ID NO: 92 - protein, 6 aa, Artificial Sequence
  Artificial peptide sequence
DKPWLV

SEQ ID NO: 93 - protein, 6 aa, Artificial Sequence
  Artificial peptide sequence
DKPWVL

SEQ ID NO: 94 - protein, 6 aa, Artificial Sequence
  Artificial peptide sequence
DKPWVI

SEQ ID NO: 95 - protein, 6 aa, Artificial Sequence
  Artificial peptide sequence
DKPWVV

SEQ ID NO: 96 - protein, 6 aa, Artificial Sequence
  Artificial peptide sequence
DRPYIL

SEQ ID NO: 97 - protein, 6 aa, Artificial Sequence
  Artificial peptide sequence
DRPYII

SEQ ID NO: 98 - protein, 6 aa, Artificial Sequence
  Artificial peptide sequence
DRPYIV

SEQ ID NO: 99 - protein, 6 aa, Artificial Sequence
  Artificial peptide sequence
DRPYLL

SEQ ID NO: 100 - protein, 6 aa, Artificial Sequence
  Artificial peptide sequence
DRPYLI

SEQ ID NO: 101 - protein, 6 aa, Artificial Sequence
  Artificial peptide sequence
DRPYLV

SEQ ID NO: 102 - protein, 6 aa, Artificial Sequence
  Artificial peptide sequence
DRPYVL

SEQ ID NO: 103 - protein, 6 aa, Artificial Sequence
  Artificial peptide sequence
DRPYVI

SEQ ID NO: 104 - protein, 6 aa, Artificial Sequence
  Artificial peptide sequence
DRPYVV

SEQ ID NO: 105 - protein, 6 aa, Artificial Sequence
  Artificial peptide sequence
DRPWIL

SEQ ID NO: 106 - protein, 6 aa, Artificial Sequence
  Artificial peptide sequence
DRPWII

SEQ ID NO: 107 - protein, 6 aa, Artificial Sequence
  Artificial peptide sequence
DRPWIV

SEQ ID NO: 108 - protein, 6 aa, Artificial Sequence
  Artificial peptide sequence
DRPWLL

SEQ ID NO: 109 - protein, 6 aa, Artificial Sequence
  Artificial peptide sequence
DRPWLI

SEQ ID NO: 110 - protein, 6 aa, Artificial Sequence
  Artificial peptide sequence
DRPWLV

SEQ ID NO: 111 - protein, 6 aa, Artificial Sequence
  Artificial peptide sequence
DRPWVL

SEQ ID NO: 112 - protein, 6 aa, Artificial Sequence
  Artificial peptide sequence
DRPWVI

SEQ ID NO: 113 - protein, 6 aa, Artificial Sequence
  Artificial peptide sequence
DRPWVV

SEQ ID NO: 114 - protein, 6 aa, Artificial Sequence
  Artificial peptide sequence
EKPYIL

SEQ ID NO: 115 - protein, 6 aa, Artificial Sequence
  Artificial peptide sequence
EKPYII

SEQ ID NO: 116 - protein, 6 aa, Artificial Sequence
  Artificial peptide sequence
EKPYIV

SEQ ID NO: 117 - protein, 6 aa, Artificial Sequence
  Artificial peptide sequence
EKPYLL

SEQ ID NO: 118 - protein, 6 aa, Artificial Sequence
  Artificial peptide sequence
EKPYLI

SEQ ID NO: 119 - protein, 6 aa, Artificial Sequence
  Artificial peptide sequence
EKPYLV

SEQ ID NO: 120 - protein, 6 aa, Artificial Sequence
  Artificial peptide sequence
EKPYVL

SEQ ID NO: 121 - protein, 6 aa, Artificial Sequence
  Artificial peptide sequence
EKPYVI

SEQ ID NO: 122 - protein, 6 aa, Artificial Sequence
  Artificial peptide sequence
EKPYVV

SEQ ID NO: 123 - protein, 6 aa, Artificial Sequence
  Artificial peptide sequence
EKPWIL

SEQ ID NO: 124 - protein, 6 aa, Artificial Sequence
  Artificial peptide sequence
EKPWII

SEQ ID NO: 125 - protein, 6 aa, Artificial Sequence
  Artificial peptide sequence
EKPWIV

SEQ ID NO: 126 - protein, 6 aa, Artificial Sequence
  Artificial peptide sequence
EKPWLL

SEQ ID NO: 127 - protein, 6 aa, Artificial Sequence
  Artificial peptide sequence
EKPWLI

SEQ ID NO: 128 - protein, 6 aa, Artificial Sequence
  Artificial peptide sequence
EKPWLV

SEQ ID NO: 129 - protein, 6 aa, Artificial Sequence
  Artificial peptide sequence
EKPWVL

SEQ ID NO: 130 - protein, 6 aa, Artificial Sequence
  Artificial peptide sequence
EKPWVI

SEQ ID NO: 131 - protein, 6 aa, Artificial Sequence
  Artificial peptide sequence
EKPWVV

SEQ ID NO: 132 - protein, 6 aa, Artificial Sequence
  Artificial peptide sequence
ERPYIL

SEQ ID NO: 133 - protein, 6 aa, Artificial Sequence
  Artificial peptide sequence
ERPYII

SEQ ID NO: 134 - protein, 6 aa, Artificial Sequence
  Artificial peptide sequence
ERPYIV

SEQ ID NO: 135 - protein, 6 aa, Artificial Sequence
  Artificial peptide sequence
ERPYLL

SEQ ID NO: 136 - protein, 6 aa, Artificial Sequence
  Artificial peptide sequence
ERPYLI

SEQ ID NO: 137 - protein, 6 aa, Artificial Sequence
  Artificial peptide sequence
ERPYLV

SEQ ID NO: 138 - protein, 6 aa, Artificial Sequence
  Artificial peptide sequence
ERPYVL

SEQ ID NO: 139 - protein, 6 aa, Artificial Sequence
  Artificial peptide sequence
ERPYVI

SEQ ID NO: 140 - protein, 6 aa, Artificial Sequence
  Artificial peptide sequence
ERPYVV

SEQ ID NO: 141 - protein, 6 aa, Artificial Sequence
  Artificial peptide sequence
ERPWIL

SEQ ID NO: 142 - protein, 6 aa, Artificial Sequence
  Artificial peptide sequence
ERPWII

SEQ ID NO: 143 - protein, 6 aa, Artificial Sequence
  Artificial peptide sequence
ERPWIV

SEQ ID NO: 144 - protein, 6 aa, Artificial Sequence
  Artificial peptide sequence
ERPWLL

SEQ ID NO: 145 - protein, 6 aa, Artificial Sequence
  Artificial peptide sequence
ERPWLI

SEQ ID NO: 146 - protein, 6 aa, Artificial Sequence
  Artificial peptide sequence
ERPWLV

SEQ ID NO: 147 - protein, 6 aa, Artificial Sequence
  Artificial peptide sequence
ERPWVL

SEQ ID NO: 148 - protein, 6 aa, Artificial Sequence
  Artificial peptide sequence
ERPWVI

SEQ ID NO: 149 - protein, 6 aa, Artificial Sequence
  Artificial peptide sequence
ERPWVV

SEQ ID NO: 150 - protein, 6 aa, Artificial Sequence
  Artificial peptide sequence
RKPYIL

SEQ ID NO: 151 - protein, 6 aa, Artificial Sequence
  Artificial peptide sequence
RKPYII

SEQ ID NO: 152 - protein, 6 aa, Artificial Sequence
  Artificial peptide sequence
RKPYIV

SEQ ID NO: 153 - protein, 6 aa, Artificial Sequence
  Artificial peptide sequence
RKPYLL

SEQ ID NO: 154 - protein, 6 aa, Artificial Sequence
  Artificial peptide sequence
RKPYLI

SEQ ID NO: 155 - protein, 6 aa, Artificial Sequence
  Artificial peptide sequence
RKPYLV

SEQ ID NO: 156 - protein, 6 aa, Artificial Sequence
  Artificial peptide sequence
RKPYVL

SEQ ID NO: 157 - protein, 6 aa, Artificial Sequence
  Artificial peptide sequence
RKPYVI

SEQ ID NO: 158 - protein, 6 aa, Artificial Sequence
  Artificial peptide sequence
RKPYVV

SEQ ID NO: 159 - protein, 6 aa, Artificial Sequence
  Artificial peptide sequence
RKPWIL

SEQ ID NO: 160 - protein, 6 aa, Artificial Sequence
  Artificial peptide sequence
RKPWII

SEQ ID NO: 161 - protein, 6 aa, Artificial Sequence
  Artificial peptide sequence
RKPWIV

SEQ ID NO: 162 - protein, 6 aa, Artificial Sequence
  Artificial peptide sequence
RKPWLL

SEQ ID NO: 163 - protein, 6 aa, Artificial Sequence
  Artificial peptide sequence
RKPWLI

SEQ ID NO: 164 - protein, 6 aa, Artificial Sequence
  Artificial peptide sequence
RKPWLV

SEQ ID NO: 165 - protein, 6 aa, Artificial Sequence
  Artificial peptide sequence
RKPWVL

SEQ ID NO: 166 - protein, 6 aa, Artificial Sequence
  Artificial peptide sequence
RKPWVI

SEQ ID NO: 167 - protein, 6 aa, Artificial Sequence
  Artificial peptide sequence
RKPWVV

SEQ ID NO: 168 - protein, 6 aa, Artificial Sequence
  Artificial peptide sequence
RRPYII

SEQ ID NO: 169 - protein, 6 aa, Artificial Sequence
  Artificial peptide sequence
RRPYIV

SEQ ID NO: 170 - protein, 6 aa, Artificial Sequence
  Artificial peptide sequence
RRPYLL

SEQ ID NO: 171 - protein, 6 aa, Artificial Sequence
  Artificial peptide sequence
RRPYLI

SEQ ID NO: 172 - protein, 6 aa, Artificial Sequence
  Artificial peptide sequence
RRPYLV

SEQ ID NO: 173 - protein, 6 aa, Artificial Sequence
  Artificial peptide sequence
RRPYVL

SEQ ID NO: 174 - protein, 6 aa, Artificial Sequence
  Artificial peptide sequence
RRPYVI

SEQ ID NO: 175 - protein, 6 aa, Artificial Sequence
  Artificial peptide sequence
RRPYVV

SEQ ID NO: 176 - protein, 6 aa, Artificial Sequence
  Artificial peptide sequence
RRPWII

SEQ ID NO: 177 - protein, 6 aa, Artificial Sequence
  Artificial peptide sequence
RRPWIV

SEQ ID NO: 178 - protein, 6 aa, Artificial Sequence
  Artificial peptide sequence
RRPWLL

SEQ ID NO: 179 - protein, 6 aa, Artificial Sequence
  Artificial peptide sequence
RRPWLI

SEQ ID NO: 180 - protein, 6 aa, Artificial Sequence
  Artificial peptide sequence
RRPWLV

SEQ ID NO: 181 - protein, 6 aa, Artificial Sequence
  Artificial peptide sequence
RRPWVL

SEQ ID NO: 182 - protein, 6 aa, Artificial Sequence
  Artificial peptide sequence
RRPWVI

SEQ ID NO: 183 - protein, 6 aa, Artificial Sequence
  Artificial peptide sequence
RRPWVV

SEQ ID NO: 184 - protein, 6 aa, Artificial Sequence
  Artificial peptide sequence
GKPYIL

SEQ ID NO: 185 - protein, 6 aa, Artificial Sequence
  Artificial peptide sequence
GKPYII

SEQ ID NO: 186 - protein, 6 aa, Artificial Sequence
  Artificial peptide sequence
GKPYIV

SEQ ID NO: 187 - protein, 6 aa, Artificial Sequence
  Artificial peptide sequence
GKPYLL

SEQ ID NO: 188 - protein, 6 aa, Artificial Sequence
  Artificial peptide sequence
GKPYLI

SEQ ID NO: 189 - protein, 6 aa, Artificial Sequence
  Artificial peptide sequence
GKPYLV

SEQ ID NO: 190 - protein, 6 aa, Artificial Sequence
  Artificial peptide sequence
GKPYVL

SEQ ID NO: 191 - protein, 6 aa, Artificial Sequence
  Artificial peptide sequence
GKPYVI

SEQ ID NO: 192 - protein, 6 aa, Artificial Sequence
  Artificial peptide sequence
GKPYVV

SEQ ID NO: 193 - protein, 6 aa, Artificial Sequence
  Artificial peptide sequence
GKPWIL

SEQ ID NO: 194 - protein, 6 aa, Artificial Sequence
  Artificial peptide sequence
GKPWII

SEQ ID NO: 195 - protein, 6 aa, Artificial Sequence
  Artificial peptide sequence
GKPWIV

SEQ ID NO: 196 - protein, 6 aa, Artificial Sequence
  Artificial peptide sequence
GKPWLL

SEQ ID NO: 197 - protein, 6 aa, Artificial Sequence
  Artificial peptide sequence
GKPWLI

SEQ ID NO: 198 - protein, 6 aa, Artificial Sequence
  Artificial peptide sequence
GKPWLV

SEQ ID NO: 199 - protein, 6 aa, Artificial Sequence
  Artificial peptide sequence
GKPWVL

SEQ ID NO: 200 - protein, 6 aa, Artificial Sequence
  Artificial peptide sequence
GKPWVI

SEQ ID NO: 201 - protein, 6 aa, Artificial Sequence
  Artificial peptide sequence
GKPWVV

SEQ ID NO: 202 - protein, 6 aa, Artificial Sequence
  Artificial peptide sequence
GRPYIL

SEQ ID NO: 203 - protein, 6 aa, Artificial Sequence
  Artificial peptide sequence
GRPYII

SEQ ID NO: 204 - protein, 6 aa, Artificial Sequence
  Artificial peptide sequence
GRPYIV

SEQ ID NO: 205 - protein, 6 aa, Artificial Sequence
  Artificial peptide sequence
GRPYLL

SEQ ID NO: 206 - protein, 6 aa, Artificial Sequence
  Artificial peptide sequence
GRPYLI

SEQ ID NO: 207 - protein, 6 aa, Artificial Sequence
  Artificial peptide sequence
GRPYLV

SEQ ID NO: 208 - protein, 6 aa, Artificial Sequence
  Artificial peptide sequence
GRPYVL

SEQ ID NO: 209 - protein, 6 aa, Artificial Sequence
  Artificial peptide sequence
GRPYVI

SEQ ID NO: 210 - protein, 6 aa, Artificial Sequence
  Artificial peptide sequence
GRPYVV

SEQ ID NO: 211 - protein, 6 aa, Artificial Sequence
  Artificial peptide sequence
GRPWIL

SEQ ID NO: 212 - protein, 6 aa, Artificial Sequence
  Artificial peptide sequence
GRPWII

SEQ ID NO: 213 - protein, 6 aa, Artificial Sequence
  Artificial peptide sequence
GRPWIV

SEQ ID NO: 214 - protein, 6 aa, Artificial Sequence
  Artificial peptide sequence
GRPWLL

SEQ ID NO: 215 - protein, 6 aa, Artificial Sequence
  Artificial peptide sequence
GRPWLI

SEQ ID NO: 216 - protein, 6 aa, Artificial Sequence
  Artificial peptide sequence
GRPWLV

SEQ ID NO: 217 - protein, 6 aa, Artificial Sequence
  Artificial peptide sequence
GRPWVL

SEQ ID NO: 218 - protein, 6 aa, Artificial Sequence
  Artificial peptide sequence
GRPWVI

SEQ ID NO: 219 - protein, 6 aa, Artificial Sequence
  Artificial peptide sequence
GRPWVV

SEQ ID NO: 220 - protein, 7 aa, Artificial Sequence
  Artificial peptide sequence
SDKPYIL

SEQ ID NO: 221 - protein, 7 aa, Artificial Sequence
  Artificial peptide sequence
SDKPYII

SEQ ID NO: 222 - protein, 7 aa, Artificial Sequence
  Artificial peptide sequence
SDKPYIV

SEQ ID NO: 223 - protein, 7 aa, Artificial Sequence
  Artificial peptide sequence
SDKPYLL

SEQ ID NO: 224 - protein, 7 aa, Artificial Sequence
  Artificial peptide sequence
SDKPYLI

SEQ ID NO: 225 - protein, 7 aa, Artificial Sequence
  Artificial peptide sequence
SDKPYLV

SEQ ID NO: 226 - protein, 7 aa, Artificial Sequence
  Artificial peptide sequence
SDKPYVL

SEQ ID NO: 227 - protein, 7 aa, Artificial Sequence
  Artificial peptide sequence
SDKPYVI

SEQ ID NO: 228 - protein, 7 aa, Artificial Sequence
  Artificial peptide sequence
SDKPYVV

SEQ ID NO: 229 - protein, 7 aa, Artificial Sequence
  Artificial peptide sequence
SDKPWIL

SEQ ID NO: 230 - protein, 7 aa, Artificial Sequence
  Artificial peptide sequence
SDKPWII

SEQ ID NO: 231 - protein, 7 aa, Artificial Sequence
  Artificial peptide sequence
SDKPWIV

SEQ ID NO: 232 - protein, 7 aa, Artificial Sequence
  Artificial peptide sequence
SDKPWLL

SEQ ID NO: 233 - protein, 7 aa, Artificial Sequence
  Artificial peptide sequence
SDKPWLI

SEQ ID NO: 234 - protein, 7 aa, Artificial Sequence
  Artificial peptide sequence
SDKPWLV

SEQ ID NO: 235 - protein, 7 aa, Artificial Sequence
  Artificial peptide sequence
SDKPWVL

SEQ ID NO: 236 - protein, 7 aa, Artificial Sequence
  Artificial peptide sequence
SDKPWVI

SEQ ID NO: 237 - protein, 7 aa, Artificial Sequence
  Artificial peptide sequence
SDKPWVV

SEQ ID NO: 238 - protein, 7 aa, Artificial Sequence
  Artificial peptide sequence
SDRPYIL

SEQ ID NO: 239 - protein, 7 aa, Artificial Sequence
  Artificial peptide sequence
SDRPYII

SEQ ID NO: 240 - protein, 7 aa, Artificial Sequence
  Artificial peptide sequence
SDRPYIV

SEQ ID NO: 241 - protein, 7 aa, Artificial Sequence
  Artificial peptide sequence
SDRPYLL

SEQ ID NO: 242 - protein, 7 aa, Artificial Sequence
  Artificial peptide sequence
SDRPYLI

SEQ ID NO: 243 - protein, 7 aa, Artificial Sequence
  Artificial peptide sequence
SDRPYLV

SEQ ID NO: 244 - protein, 7 aa, Artificial Sequence
  Artificial peptide sequence
SDRPYVL

SEQ ID NO: 245 - protein, 7 aa, Artificial Sequence
  Artificial peptide sequence
SDRPYVI

SEQ ID NO: 246 - protein, 7 aa, Artificial Sequence
  Artificial peptide sequence
SDRPYVV

SEQ ID NO: 247 - protein, 7 aa, Artificial Sequence
  Artificial peptide sequence
SDRPWIL

SEQ ID NO: 248 - protein, 7 aa, Artificial Sequence
  Artificial peptide sequence
SDRPWII

SEQ ID NO: 249 - protein, 7 aa, Artificial Sequence
  Artificial peptide sequence
SDRPWIV

SEQ ID NO: 250 - protein, 7 aa, Artificial Sequence
  Artificial peptide sequence
SDRPWLL

SEQ ID NO: 251 - protein, 7 aa, Artificial Sequence
  Artificial peptide sequence
SDRPWLI

SEQ ID NO: 252 - protein, 7 aa, Artificial Sequence
  Artificial peptide sequence
SDRPWLV

SEQ ID NO: 253 - protein, 7 aa, Artificial Sequence
  Artificial peptide sequence
SDRPWVL

SEQ ID NO: 254 - protein, 7 aa, Artificial Sequence
  Artificial peptide sequence
SDRPWVI

SEQ ID NO: 255 - protein, 7 aa, Artificial Sequence
  Artificial peptide sequence
SDRPWVV

SEQ ID NO: 256 - protein, 7 aa, Artificial Sequence
  Artificial peptide sequence
SEKPYIL

SEQ ID NO: 257 - protein, 7 aa, Artificial Sequence
  Artificial peptide sequence
SEKPYII

SEQ ID NO: 258 - protein, 7 aa, Artificial Sequence
  Artificial peptide sequence
SEKPYIV

SEQ ID NO: 259 - protein, 7 aa, Artificial Sequence
  Artificial peptide sequence
SEKPYLL

SEQ ID NO: 260 - protein, 7 aa, Artificial Sequence
  Artificial peptide sequence
SEKPYLI

SEQ ID NO: 261 - protein, 7 aa, Artificial Sequence
  Artificial peptide sequence
SEKPYLV

SEQ ID NO: 262 - protein, 7 aa, Artificial Sequence
  Artificial peptide sequence
SEKPYVL

SEQ ID NO: 263 - protein, 7 aa, Artificial Sequence
  Artificial peptide sequence
SEKPYVI

SEQ ID NO: 264 - protein, 7 aa, Artificial Sequence
  Artificial peptide sequence
SEKPYVV

SEQ ID NO: 265 - protein, 7 aa, Artificial Sequence
  Artificial peptide sequence
SEKPWIL

SEQ ID NO: 266 - protein, 7 aa, Artificial Sequence
  Artificial peptide sequence
SEKPWII

SEQ ID NO: 267 - protein, 7 aa, Artificial Sequence
  Artificial peptide sequence
SEKPWIV

SEQ ID NO: 268 - protein, 7 aa, Artificial Sequence
  Artificial peptide sequence
SEKPWLL

SEQ ID NO: 269 - protein, 7 aa, Artificial Sequence
  Artificial peptide sequence
SEKPWLI

SEQ ID NO: 270 - protein, 7 aa, Artificial Sequence
  Artificial peptide sequence
SEKPWLV

SEQ ID NO: 271 - protein, 7 aa, Artificial Sequence
  Artificial peptide sequence
SEKPWVL

SEQ ID NO: 272 - protein, 7 aa, Artificial Sequence
  Artificial peptide sequence
SEKPWVI

SEQ ID NO: 273 - protein, 7 aa, Artificial Sequence
  Artificial peptide sequence
SEKPWVV

SEQ ID NO: 274 - protein, 7 aa, Artificial Sequence
  Artificial peptide sequence
SERPYIL

SEQ ID NO: 275 - protein, 7 aa, Artificial Sequence
  Artificial peptide sequence
SERPYII

SEQ ID NO: 276 - protein, 7 aa, Artificial Sequence
  Artificial peptide sequence
SERPYIV

SEQ ID NO: 277 - protein, 7 aa, Artificial Sequence
  Artificial peptide sequence
SERPYLL

SEQ ID NO: 278 - protein, 7 aa, Artificial Sequence
  Artificial peptide sequence
SERPYLI

SEQ ID NO: 279 - protein, 7 aa, Artificial Sequence
  Artificial peptide sequence
SERPYLV

SEQ ID NO: 280 - protein, 7 aa, Artificial Sequence
  Artificial peptide sequence
SERPYVL

SEQ ID NO: 281 - protein, 7 aa, Artificial Sequence
  Artificial peptide sequence
SERPYVI

SEQ ID NO: 282 - protein, 7 aa, Artificial Sequence
  Artificial peptide sequence
SERPYVV

SEQ ID NO: 283 - protein, 7 aa, Artificial Sequence
  Artificial peptide sequence
SERPWIL

SEQ ID NO: 284 - protein, 7 aa, Artificial Sequence
  Artificial peptide sequence
SERPWII

SEQ ID NO: 285 - protein, 7 aa, Artificial Sequence
  Artificial peptide sequence
SERPWIV

SEQ ID NO: 286 - protein, 7 aa, Artificial Sequence
  Artificial peptide sequence
SERPWLL

SEQ ID NO: 287 - protein, 7 aa, Artificial Sequence
  Artificial peptide sequence
SERPWLI

SEQ ID NO: 288 - protein, 7 aa, Artificial Sequence
  Artificial peptide sequence
SERPWLV

SEQ ID NO: 289 - protein, 7 aa, Artificial Sequence
  Artificial peptide sequence
SERPWVL

SEQ ID NO: 290 - protein, 7 aa, Artificial Sequence
  Artificial peptide sequence
SERPWVI

SEQ ID NO: 291 - protein, 7 aa, Artificial Sequence
  Artificial peptide sequence
SERPWVV

SEQ ID NO: 292 - protein, 7 aa, Artificial Sequence
  Artificial peptide sequence
TDKPYIL

SEQ ID NO: 293 - protein, 7 aa, Artificial Sequence
  Artificial peptide sequence
TDKPYII

SEQ ID NO: 294 - protein, 7 aa, Artificial Sequence
  Artificial peptide sequence
TDKPYIV

SEQ ID NO: 295 - protein, 7 aa, Artificial Sequence
  Artificial peptide sequence
TDKPYLL

SEQ ID NO: 296 - protein, 7 aa, Artificial Sequence
  Artificial peptide sequence
TDKPYLI

SEQ ID NO: 297 - protein, 7 aa, Artificial Sequence
  Artificial peptide sequence
TDKPYLV

SEQ ID NO: 298 - protein, 7 aa, Artificial Sequence
  Artificial peptide sequence
TDKPYVL

SEQ ID NO: 299 - protein, 7 aa, Artificial Sequence
  Artificial peptide sequence
TDKPYVI

SEQ ID NO: 300 - protein, 7 aa, Artificial Sequence
  Artificial peptide sequence
TDKPYVV

SEQ ID NO: 301 - protein, 7 aa, Artificial Sequence
  Artificial peptide sequence
TDKPWIL

SEQ ID NO: 302 - protein, 7 aa, Artificial Sequence
  Artificial peptide sequence
TDKPWII

SEQ ID NO: 303 - protein, 7 aa, Artificial Sequence
  Artificial peptide sequence
TDKPWIV

SEQ ID NO: 304 - protein, 7 aa, Artificial Sequence
  Artificial peptide sequence
TDKPWLL

SEQ ID NO: 305 - protein, 7 aa, Artificial Sequence
  Artificial peptide sequence
TDKPWLI

SEQ ID NO: 306 - protein, 7 aa, Artificial Sequence
  Artificial peptide sequence
TDKPWLV

SEQ ID NO: 307 - protein, 7 aa, Artificial Sequence
  Artificial peptide sequence
TDKPWVL

SEQ ID NO: 308 - protein, 7 aa, Artificial Sequence
  Artificial peptide sequence
TDKPWVI

SEQ ID NO: 309 - protein, 7 aa, Artificial Sequence
  Artificial peptide sequence
TDKPWVV

SEQ ID NO: 310 - protein, 7 aa, Artificial Sequence
  Artificial peptide sequence
TDRPYIL

SEQ ID NO: 311 - protein, 7 aa, Artificial Sequence
  Artificial peptide sequence
TDRPYII

SEQ ID NO: 312 - protein, 7 aa, Artificial Sequence
  Artificial peptide sequence
TDRPYIV

SEQ ID NO: 313 - protein, 7 aa, Artificial Sequence
  Artificial peptide sequence
TDRPYLL

SEQ ID NO: 314 - protein, 7 aa, Artificial Sequence
  Artificial peptide sequence
TDRPYLI

SEQ ID NO: 315 - protein, 7 aa, Artificial Sequence
  Artificial peptide sequence
TDRPYLV

SEQ ID NO: 316 - protein, 7 aa, Artificial Sequence
  Artificial peptide sequence
TDRPYVL

SEQ ID NO: 317 - protein, 7 aa, Artificial Sequence
  Artificial peptide sequence
TDRPYVI

SEQ ID NO: 318 - protein, 7 aa, Artificial Sequence
  Artificial peptide sequence
TDRPYVV

SEQ ID NO: 319 - protein, 7 aa, Artificial Sequence
  Artificial peptide sequence
TDRPWIL

SEQ ID NO: 320 - protein, 7 aa, Artificial Sequence
  Artificial peptide sequence
TDRPWII

SEQ ID NO: 321 - protein, 7 aa, Artificial Sequence
  Artificial peptide sequence
TDRPWIV

SEQ ID NO: 322 - protein, 7 aa, Artificial Sequence
  Artificial peptide sequence
TDRPWLL

SEQ ID NO: 323 - protein, 7 aa, Artificial Sequence
  Artificial peptide sequence
TDRPWLI

SEQ ID NO: 324 - protein, 7 aa, Artificial Sequence
  Artificial peptide sequence
TDRPWLV

SEQ ID NO: 325 - protein, 7 aa, Artificial Sequence
  Artificial peptide sequence
TDRPWVL

SEQ ID NO: 326 - protein, 7 aa, Artificial Sequence
  Artificial peptide sequence
TDRPWVI

SEQ ID NO: 327 - protein, 7 aa, Artificial Sequence
  Artificial peptide sequence
TDRPWVV

SEQ ID NO: 328 - protein, 7 aa, Artificial Sequence
  Artificial peptide sequence
TEKPYIL

SEQ ID NO: 329 - protein, 7 aa, Artificial Sequence
  Artificial peptide sequence
TEKPYII

SEQ ID NO: 330 - protein, 7 aa, Artificial Sequence
  Artificial peptide sequence
TEKPYIV

SEQ ID NO: 331 - protein, 7 aa, Artificial Sequence
  Artificial peptide sequence
TEKPYLL

SEQ ID NO: 332 - protein, 7 aa, Artificial Sequence
  Artificial peptide sequence
TEKPYLI

SEQ ID NO: 333 - protein, 7 aa, Artificial Sequence
  Artificial peptide sequence
TEKPYLV

SEQ ID NO: 334 - protein, 7 aa, Artificial Sequence
  Artificial peptide sequence
TEKPYVL

SEQ ID NO: 335 - protein, 7 aa, Artificial Sequence
  Artificial peptide sequence
TEKPYVI

SEQ ID NO: 336 - protein, 7 aa, Artificial Sequence
  Artificial peptide sequence
TEKPYVV

SEQ ID NO: 337 - protein, 7 aa, Artificial Sequence
  Artificial peptide sequence
TEKPWIL

SEQ ID NO: 338 - protein, 7 aa, Artificial Sequence
  Artificial peptide sequence
TEKPWII

SEQ ID NO: 339 - protein, 7 aa, Artificial Sequence
  Artificial peptide sequence
TEKPWIV

SEQ ID NO: 340 - protein, 7 aa, Artificial Sequence
  Artificial peptide sequence
TEKPWLL

SEQ ID NO: 341 - protein, 7 aa, Artificial Sequence
  Artificial peptide sequence
TEKPWLI

SEQ ID NO: 342 - protein, 7 aa, Artificial Sequence
  Artificial peptide sequence
TEKPWLV

SEQ ID NO: 343 - protein, 7 aa, Artificial Sequence
  Artificial peptide sequence
TEKPWVL

SEQ ID NO: 344 - protein, 7 aa, Artificial Sequence
  Artificial peptide sequence
TEKPWVI

SEQ ID NO: 345 - protein, 7 aa, Artificial Sequence
  Artificial peptide sequence
TEKPWVV

SEQ ID NO: 346 - protein, 7 aa, Artificial Sequence
  Artificial peptide sequence
TERPYIL

SEQ ID NO: 347 - protein, 7 aa, Artificial Sequence
  Artificial peptide sequence
TERPYII

SEQ ID NO: 348 - protein, 7 aa, Artificial Sequence
  Artificial peptide sequence
TERPYIV

SEQ ID NO: 349 - protein, 7 aa, Artificial Sequence
  Artificial peptide sequence
TERPYLL

SEQ ID NO: 350 - protein, 7 aa, Artificial Sequence
  Artificial peptide sequence
TERPYLI

SEQ ID NO: 351 - protein, 7 aa, Artificial Sequence
  Artificial peptide sequence
TERPYLV

SEQ ID NO: 352 - protein, 7 aa, Artificial Sequence
  Artificial peptide sequence
TERPYVL

SEQ ID NO: 353 - protein, 7 aa, Artificial Sequence
  Artificial peptide sequence
TERPYVI

SEQ ID NO: 354 - protein, 7 aa, Artificial Sequence
  Artificial peptide sequence
TERPYVV

SEQ ID NO: 355 - protein, 7 aa, Artificial Sequence
  Artificial peptide sequence
TERPWIL

SEQ ID NO: 356 - protein, 7 aa, Artificial Sequence
  Artificial peptide sequence
TERPWII

SEQ ID NO: 357 - protein, 7 aa, Artificial Sequence
  Artificial peptide sequence
TERPWIV

SEQ ID NO: 358 - protein, 7 aa, Artificial Sequence
  Artificial peptide sequence
TERPWLL

SEQ ID NO: 359 - protein, 7 aa, Artificial Sequence
  Artificial peptide sequence
TERPWLI

SEQ ID NO: 360 - protein, 7 aa, Artificial Sequence
  Artificial peptide sequence
TERPWLV

SEQ ID NO: 361 - protein, 7 aa, Artificial Sequence
  Artificial peptide sequence
TERPWVL

SEQ ID NO: 362 - protein, 7 aa, Artificial Sequence
  Artificial peptide sequence
TERPWVI

SEQ ID NO: 363 - protein, 7 aa, Artificial Sequence
  Artificial peptide sequence
TERPWVV

SEQ ID NO: 364 - protein, 8 aa, Artificial Sequence
  Artificial peptide sequence
ASDKPYII

SEQ ID NO: 365 - protein, 8 aa, Artificial Sequence
  Artificial peptide sequence
ASDKPYIV

SEQ ID NO: 366 - protein, 8 aa, Artificial Sequence
  Artificial peptide sequence
ASDKPYLL

SEQ ID NO: 367 - protein, 8 aa, Artificial Sequence
  Artificial peptide sequence
ASDKPYLI

SEQ ID NO: 368 - protein, 8 aa, Artificial Sequence
  Artificial peptide sequence
ASDKPYLV

SEQ ID NO: 369 - protein, 8 aa, Artificial Sequence
  Artificial peptide sequence
ASDKPYVL

SEQ ID NO: 370 - protein, 8 aa, Artificial Sequence
  Artificial peptide sequence
ASDKPYVI

SEQ ID NO: 371 - protein, 8 aa, Artificial Sequence
  Artificial peptide sequence
ASDKPYVV

SEQ ID NO: 372 - protein, 8 aa, Artificial Sequence
  Artificial peptide sequence
ASDKPWIL

SEQ ID NO: 373 - protein, 8 aa, Artificial Sequence
  Artificial peptide sequence
ASDKPWII

SEQ ID NO: 374 - protein, 8 aa, Artificial Sequence
  Artificial peptide sequence
ASDKPWIV

SEQ ID NO: 375 - protein, 8 aa, Artificial Sequence
  Artificial peptide sequence
ASDKPWLL

SEQ ID NO: 376 - protein, 8 aa, Artificial Sequence
  Artificial peptide sequence
ASDKPWLI

SEQ ID NO: 377 - protein, 8 aa, Artificial Sequence
  Artificial peptide sequence
ASDKPWLV

SEQ ID NO: 378 - protein, 8 aa, Artificial Sequence
  Artificial peptide sequence
ASDKPWVL

SEQ ID NO: 379 - protein, 8 aa, Artificial Sequence
  Artificial peptide sequence
ASDKPWVI

SEQ ID NO: 380 - protein, 8 aa, Artificial Sequence
  Artificial peptide sequence
ASDKPWVV

SEQ ID NO: 381 - protein, 8 aa, Artificial Sequence
  Artificial peptide sequence
ASDRPYIL

SEQ ID NO: 382 - protein, 8 aa, Artificial Sequence
  Artificial peptide sequence
ASDRPYII

SEQ ID NO: 383 - protein, 8 aa, Artificial Sequence
  Artificial peptide sequence
ASDRPYIV

SEQ ID NO: 384 - protein, 8 aa, Artificial Sequence
  Artificial peptide sequence
ASDRPYLL

SEQ ID NO: 385 - protein, 8 aa, Artificial Sequence
  Artificial peptide sequence
ASDRPYLI

SEQ ID NO: 386 - protein, 8 aa, Artificial Sequence
  Artificial peptide sequence
ASDRPYLV

SEQ ID NO: 387 - protein, 8 aa, Artificial Sequence
  Artificial peptide sequence
ASDRPYVL

SEQ ID NO: 388 - protein, 8 aa, Artificial Sequence
  Artificial peptide sequence
ASDRPYVI

SEQ ID NO: 389 - protein, 8 aa, Artificial Sequence
  Artificial peptide sequence
ASDRPYVV

SEQ ID NO: 390 - protein, 8 aa, Artificial Sequence
  Artificial peptide sequence
ASDRPWIL

SEQ ID NO: 391 - protein, 8 aa, Artificial Sequence
  Artificial peptide sequence
ASDRPWII

SEQ ID NO: 392 - protein, 8 aa, Artificial Sequence
  Artificial peptide sequence
ASDRPWIV

SEQ ID NO: 393 - protein, 8 aa, Artificial Sequence
  Artificial peptide sequence
ASDRPWLL

SEQ ID NO: 394 - protein, 8 aa, Artificial Sequence
  Artificial peptide sequence
ASDRPWLI

SEQ ID NO: 395 - protein, 8 aa, Artificial Sequence
  Artificial peptide sequence
ASDRPWLV

SEQ ID NO: 396 - protein, 8 aa, Artificial Sequence
  Artificial peptide sequence
ASDRPWVL

SEQ ID NO: 397 - protein, 8 aa, Artificial Sequence
  Artificial peptide sequence
ASDRPWVI

SEQ ID NO: 398 - protein, 8 aa, Artificial Sequence
  Artificial peptide sequence
ASDRPWVV

SEQ ID NO: 399 - protein, 8 aa, Artificial Sequence
  Artificial peptide sequence
ASEKPYIL

SEQ ID NO: 400 - protein, 8 aa, Artificial Sequence
  Artificial peptide sequence
ASEKPYII

SEQ ID NO: 401 - protein, 8 aa, Artificial Sequence
  Artificial peptide sequence
ASEKPYIV

SEQ ID NO: 402 - protein, 8 aa, Artificial Sequence
  Artificial peptide sequence
ASEKPYLL

SEQ ID NO: 403 - protein, 8 aa, Artificial Sequence
  Artificial peptide sequence
ASEKPYLI

SEQ ID NO: 404 - protein, 8 aa, Artificial Sequence
  Artificial peptide sequence
ASEKPYLV

SEQ ID NO: 405 - protein, 8 aa, Artificial Sequence
  Artificial peptide sequence
ASEKPYVL

SEQ ID NO: 406 - protein, 8 aa, Artificial Sequence
  Artificial peptide sequence
ASEKPYVI

SEQ ID NO: 407 - protein, 8 aa, Artificial Sequence
  Artificial peptide sequence
ASEKPYVV

SEQ ID NO: 408 - protein, 8 aa, Artificial Sequence
  Artificial peptide sequence
ASEKPWIL

SEQ ID NO: 409 - protein, 8 aa, Artificial Sequence
  Artificial peptide sequence
ASEKPWII

SEQ ID NO: 410 - protein, 8 aa, Artificial Sequence
  Artificial peptide sequence
ASEKPWIV

SEQ ID NO: 411 - protein, 8 aa, Artificial Sequence
  Artificial peptide sequence
ASEKPWLL

SEQ ID NO: 412 - protein, 8 aa, Artificial Sequence
  Artificial peptide sequence
ASEKPWLI

SEQ ID NO: 413 - protein, 8 aa, Artificial Sequence
  Artificial peptide sequence
ASEKPWLV

SEQ ID NO: 414 - protein, 8 aa, Artificial Sequence
  Artificial peptide sequence
ASEKPWVL

SEQ ID NO: 415 - protein, 8 aa, Artificial Sequence
  Artificial peptide sequence
ASEKPWVI

SEQ ID NO: 416 - protein, 8 aa, Artificial Sequence
  Artificial peptide sequence
ASEKPWVV

SEQ ID NO: 417 - protein, 8 aa, Artificial Sequence
  Artificial peptide sequence
ASERPYIL

SEQ ID NO: 418 - protein, 8 aa, Artificial Sequence
  Artificial peptide sequence
ASERPYII

SEQ ID NO: 419 - protein, 8 aa, Artificial Sequence
  Artificial peptide sequence
ASERPYIV

SEQ ID NO: 420 - protein, 8 aa, Artificial Sequence
  Artificial peptide sequence
ASERPYLL

SEQ ID NO: 421 - protein, 8 aa, Artificial Sequence
  Artificial peptide sequence
ASERPYLI

SEQ ID NO: 422 - protein, 8 aa, Artificial Sequence
  Artificial peptide sequence
ASERPYLV

SEQ ID NO: 423 - protein, 8 aa, Artificial Sequence
  Artificial peptide sequence
ASERPYVL

SEQ ID NO: 424 - protein, 8 aa, Artificial Sequence
  Artificial peptide sequence
ASERPYVI

SEQ ID NO: 425 - protein, 8 aa, Artificial Sequence
  Artificial peptide sequence
ASERPYVV

SEQ ID NO: 426 - protein, 8 aa, Artificial Sequence
  Artificial peptide sequence
ASERPWIL

SEQ ID NO: 427 - protein, 8 aa, Artificial Sequence
  Artificial peptide sequence
ASERPWII

SEQ ID NO: 428 - protein, 8 aa, Artificial Sequence
  Artificial peptide sequence
ASERPWIV

SEQ ID NO: 429 - protein, 8 aa, Artificial Sequence
  Artificial peptide sequence
ASERPWLL

SEQ ID NO: 430 - protein, 8 aa, Artificial Sequence
  Artificial peptide sequence
ASERPWLI

SEQ ID NO: 431 - protein, 8 aa, Artificial Sequence
  Artificial peptide sequence
ASERPWLV

SEQ ID NO: 432 - protein, 8 aa, Artificial Sequence
  Artificial peptide sequence
ASERPWVL

SEQ ID NO: 433 - protein, 8 aa, Artificial Sequence
  Artificial peptide sequence
ASERPWVI

SEQ ID NO: 434 - protein, 8 aa, Artificial Sequence
  Artificial peptide sequence
ASERPWVV

SEQ ID NO: 435 - protein, 8 aa, Artificial Sequence
  Artificial peptide sequence
ATDKPYIL

SEQ ID NO: 436 - protein, 8 aa, Artificial Sequence
  Artificial peptide sequence
ATDKPYII

SEQ ID NO: 437 - protein, 8 aa, Artificial Sequence
  Artificial peptide sequence
ATDKPYIV

SEQ ID NO: 438 - protein, 8 aa, Artificial Sequence
  Artificial peptide sequence
ATDKPYLL

SEQ ID NO: 439 - protein, 8 aa, Artificial Sequence
  Artificial peptide sequence
ATDKPYLI

SEQ ID NO: 440 - protein, 8 aa, Artificial Sequence
  Artificial peptide sequence
ATDKPYLV

SEQ ID NO: 441 - protein, 8 aa, Artificial Sequence
  Artificial peptide sequence
ATDKPYVL

SEQ ID NO: 442 - protein, 8 aa, Artificial Sequence
  Artificial peptide sequence
ATDKPYVI

SEQ ID NO: 443 - protein, 8 aa, Artificial Sequence
  Artificial peptide sequence
ATDKPYVV

SEQ ID NO: 444 - protein, 8 aa, Artificial Sequence
  Artificial peptide sequence
ATDKPWIL

SEQ ID NO: 445 - protein, 8 aa, Artificial Sequence
  Artificial peptide sequence
ATDKPWII

SEQ ID NO: 446 - protein, 8 aa, Artificial Sequence
  Artificial peptide sequence
ATDKPWIV

SEQ ID NO: 447 - protein, 8 aa, Artificial Sequence
  Artificial peptide sequence
ATDKPWLL

SEQ ID NO: 448 - protein, 8 aa, Artificial Sequence
  Artificial peptide sequence
ATDKPWLI

SEQ ID NO: 449 - protein, 8 aa, Artificial Sequence
  Artificial peptide sequence
ATDKPWLV

SEQ ID NO: 450 - protein, 8 aa, Artificial Sequence
  Artificial peptide sequence
ATDKPWVL

SEQ ID NO: 451 - protein, 8 aa, Artificial Sequence
  Artificial peptide sequence
ATDKPWVI

SEQ ID NO: 452 - protein, 8 aa, Artificial Sequence
  Artificial peptide sequence
ATDKPWVV

SEQ ID NO: 453 - protein, 8 aa, Artificial Sequence
  Artificial peptide sequence
ATDRPYIL

SEQ ID NO: 454 - protein, 8 aa, Artificial Sequence
  Artificial peptide sequence
ATDRPYII

SEQ ID NO: 455 - protein, 8 aa, Artificial Sequence
  Artificial peptide sequence
ATDRPYIV

SEQ ID NO: 456 - protein, 8 aa, Artificial Sequence
  Artificial peptide sequence
ATDRPYLL

SEQ ID NO: 457 - protein, 8 aa, Artificial Sequence
  Artificial peptide sequence
ATDRPYLI

SEQ ID NO: 458 - protein, 8 aa, Artificial Sequence
  Artificial peptide sequence
ATDRPYLV

SEQ ID NO: 459 - protein, 8 aa, Artificial Sequence
  Artificial peptide sequence
ATDRPYVL

SEQ ID NO: 460 - protein, 8 aa, Artificial Sequence
  Artificial peptide sequence
ATDRPYVI

SEQ ID NO: 461 - protein, 8 aa, Artificial Sequence
  Artificial peptide sequence
ATDRPYVV

SEQ ID NO: 462 - protein, 8 aa, Artificial Sequence
  Artificial peptide sequence
ATDRPWIL

SEQ ID NO: 463 - protein, 8 aa, Artificial Sequence
  Artificial peptide sequence
ATDRPWII

SEQ ID NO: 464 - protein, 8 aa, Artificial Sequence
  Artificial peptide sequence
ATDRPWIV

SEQ ID NO: 465 - protein, 8 aa, Artificial Sequence
  Artificial peptide sequence
ATDRPWLL

SEQ ID NO: 466 - protein, 8 aa, Artificial Sequence
  Artificial peptide sequence
ATDRPWLI

SEQ ID NO: 467 - protein, 8 aa, Artificial Sequence
  Artificial peptide sequence
ATDRPWLV

SEQ ID NO: 468 - protein, 8 aa, Artificial Sequence
  Artificial peptide sequence
ATDRPWVL

SEQ ID NO: 469 - protein, 8 aa, Artificial Sequence
  Artificial peptide sequence
ATDRPWVI

SEQ ID NO: 470 - protein, 8 aa, Artificial Sequence
  Artificial peptide sequence
ATDRPWVV

SEQ ID NO: 471 - protein, 8 aa, Artificial Sequence
  Artificial peptide sequence
ATEKPYIL

SEQ ID NO: 472 - protein, 8 aa, Artificial Sequence
  Artificial peptide sequence
ATEKPYII

SEQ ID NO: 473 - protein, 8 aa, Artificial Sequence
  Artificial peptide sequence
ATEKPYIV

SEQ ID NO: 474 - protein, 8 aa, Artificial Sequence
  Artificial peptide sequence
ATEKPYLL

SEQ ID NO: 475 - protein, 8 aa, Artificial Sequence
  Artificial peptide sequence
ATEKPYLI

SEQ ID NO: 476 - protein, 8 aa, Artificial Sequence
  Artificial peptide sequence
ATEKPYLV

SEQ ID NO: 477 - protein, 8 aa, Artificial Sequence
  Artificial peptide sequence
ATEKPYVL

SEQ ID NO: 478 - protein, 8 aa, Artificial Sequence
  Artificial peptide sequence
ATEKPYVI

SEQ ID NO: 479 - protein, 8 aa, Artificial Sequence
  Artificial peptide sequence
ATEKPYVV

SEQ ID NO: 480 - protein, 8 aa, Artificial Sequence
  Artificial peptide sequence
ATEKPWIL

SEQ ID NO: 481 - protein, 8 aa, Artificial Sequence
  Artificial peptide sequence
ATEKPWII

SEQ ID NO: 482 - protein, 8 aa, Artificial Sequence
  Artificial peptide sequence
ATEKPWIV

SEQ ID NO: 483 - protein, 8 aa, Artificial Sequence
  Artificial peptide sequence
ATEKPWLL

SEQ ID NO: 484 - protein, 8 aa, Artificial Sequence
  Artificial peptide sequence
ATEKPWLI

SEQ ID NO: 485 - protein, 8 aa, Artificial Sequence
  Artificial peptide sequence
ATEKPWLV

SEQ ID NO: 486 - protein, 8 aa, Artificial Sequence
  Artificial peptide sequence
ATEKPWVL

SEQ ID NO: 487 - protein, 8 aa, Artificial Sequence
  Artificial peptide sequence
ATEKPWVI

SEQ ID NO: 488 - protein, 8 aa, Artificial Sequence
  Artificial peptide sequence
ATEKPWVV

SEQ ID NO: 489 - protein, 8 aa, Artificial Sequence
  Artificial peptide sequence
ATERPYIL

SEQ ID NO: 490 - protein, 8 aa, Artificial Sequence
  Artificial peptide sequence
ATERPYII

SEQ ID NO: 491 - protein, 8 aa, Artificial Sequence
  Artificial peptide sequence
ATERPYIV

SEQ ID NO: 492 - protein, 8 aa, Artificial Sequence
  Artificial peptide sequence
ATERPYLL

SEQ ID NO: 493 - protein, 8 aa, Artificial Sequence
  Artificial peptide sequence
ATERPYLI

SEQ ID NO: 494 - protein, 8 aa, Artificial Sequence
  Artificial peptide sequence
ATERPYLV

SEQ ID NO: 495 - protein, 8 aa, Artificial Sequence
  Artificial peptide sequence
ATERPYVL

SEQ ID NO: 496 - protein, 8 aa, Artificial Sequence
  Artificial peptide sequence
ATERPYVI

SEQ ID NO: 497 - protein, 8 aa, Artificial Sequence
  Artificial peptide sequence
ATERPYVV

SEQ ID NO: 498 - protein, 8 aa, Artificial Sequence
  Artificial peptide sequence
ATERPWIL

SEQ ID NO: 499 - protein, 8 aa, Artificial Sequence
  Artificial peptide sequence
ATERPWII

SEQ ID NO: 500 - protein, 8 aa, Artificial Sequence
  Artificial peptide sequence
ATERPWIV

SEQ ID NO: 501 - protein, 8 aa, Artificial Sequence
  Artificial peptide sequence
ATERPWLL

SEQ ID NO: 502 - protein, 8 aa, Artificial Sequence
  Artificial peptide sequence
ATERPWLI

SEQ ID NO: 503 - protein, 8 aa, Artificial Sequence
  Artificial peptide sequence
ATERPWLV

SEQ ID NO: 504 - protein, 8 aa, Artificial Sequence
  Artificial peptide sequence
ATERPWVL

SEQ ID NO: 505 - protein, 8 aa, Artificial Sequence
  Artificial peptide sequence
ATERPWVI

SEQ ID NO: 506 - protein, 8 aa, Artificial Sequence
  Artificial peptide sequence
ATERPWVV

SEQ ID NO: 507 - protein, 8 aa, Artificial Sequence
  Artificial peptide sequence
LSDKPYIL

SEQ ID NO: 508 - protein, 8 aa, Artificial Sequence
  Artificial peptide sequence
LSDKPYII

SEQ ID NO: 509 - protein, 8 aa, Artificial Sequence
  Artificial peptide sequence
LSDKPYIV

SEQ ID NO: 510 - protein, 8 aa, Artificial Sequence
  Artificial peptide sequence
LSDKPYLL

SEQ ID NO: 511 - protein, 8 aa, Artificial Sequence
  Artificial peptide sequence
LSDKPYLI

SEQ ID NO: 512 - protein, 8 aa, Artificial Sequence
  Artificial peptide sequence
LSDKPYLV

SEQ ID NO: 513 - protein, 8 aa, Artificial Sequence
  Artificial peptide sequence
LSDKPYVL

SEQ ID NO: 514 - protein, 8 aa, Artificial Sequence
  Artificial peptide sequence
LSDKPYVI

SEQ ID NO: 515 - protein, 8 aa, Artificial Sequence
  Artificial peptide sequence
LSDKPYVV

SEQ ID NO: 516 - protein, 8 aa, Artificial Sequence
  Artificial peptide sequence
LSDKPWIL

SEQ ID NO: 517 - protein, 8 aa, Artificial Sequence
  Artificial peptide sequence
LSDKPWII

SEQ ID NO: 518 - protein, 8 aa, Artificial Sequence
  Artificial peptide sequence
LSDKPWIV

SEQ ID NO: 519 - protein, 8 aa, Artificial Sequence
  Artificial peptide sequence
LSDKPWLL

SEQ ID NO: 520 - protein, 8 aa, Artificial Sequence
  Artificial peptide sequence
LSDKPWLI

SEQ ID NO: 521 - protein, 8 aa, Artificial Sequence
  Artificial peptide sequence
LSDKPWLV

SEQ ID NO: 522 - protein, 8 aa, Artificial Sequence
  Artificial peptide sequence
LSDKPWVL

SEQ ID NO: 523 - protein, 8 aa, Artificial Sequence
  Artificial peptide sequence
LSDKPWVI

SEQ ID NO: 524 - protein, 8 aa, Artificial Sequence
  Artificial peptide sequence
LSDKPWVV

SEQ ID NO: 525 - protein, 8 aa, Artificial Sequence
  Artificial peptide sequence
LSDRPYIL

SEQ ID NO: 526 - protein, 8 aa, Artificial Sequence
  Artificial peptide sequence
LSDRPYII

SEQ ID NO: 527 - protein, 8 aa, Artificial Sequence
  Artificial peptide sequence
LSDRPYIV

SEQ ID NO: 528 - protein, 8 aa, Artificial Sequence
  Artificial peptide sequence
LSDRPYLL

SEQ ID NO: 529 - protein, 8 aa, Artificial Sequence
  Artificial peptide sequence
LSDRPYLI

SEQ ID NO: 530 - protein, 8 aa, Artificial Sequence
  Artificial peptide sequence
LSDRPYLV

SEQ ID NO: 531 - protein, 8 aa, Artificial Sequence
  Artificial peptide sequence
LSDRPYVL

SEQ ID NO: 532 - protein, 8 aa, Artificial Sequence
  Artificial peptide sequence
LSDRPYVI

SEQ ID NO: 533 - protein, 8 aa, Artificial Sequence
  Artificial peptide sequence
LSDRPYVV

SEQ ID NO: 534 - protein, 8 aa, Artificial Sequence
  Artificial peptide sequence
LSDRPWIL

SEQ ID NO: 535 - protein, 8 aa, Artificial Sequence
  Artificial peptide sequence
LSDRPWII

SEQ ID NO: 536 - protein, 8 aa, Artificial Sequence
  Artificial peptide sequence
LSDRPWIV

SEQ ID NO: 537 - protein, 8 aa, Artificial Sequence
  Artificial peptide sequence
LSDRPWLL

SEQ ID NO: 538 - protein, 8 aa, Artificial Sequence
  Artificial peptide sequence
LSDRPWLI

SEQ ID NO: 539 - protein, 8 aa, Artificial Sequence
  Artificial peptide sequence
LSDRPWLV

SEQ ID NO: 540 - protein, 8 aa, Artificial Sequence
  Artificial peptide sequence
LSDRPWVL

SEQ ID NO: 541 - protein, 8 aa, Artificial Sequence
  Artificial peptide sequence
LSDRPWVI

SEQ ID NO: 542 - protein, 8 aa, Artificial Sequence
  Artificial peptide sequence
LSDRPWVV

SEQ ID NO: 543 - protein, 8 aa, Artificial Sequence
  Artificial peptide sequence
LSEKPYIL

SEQ ID NO: 544 - protein, 8 aa, Artificial Sequence
  Artificial peptide sequence
LSEKPYII

SEQ ID NO: 545 - protein, 8 aa, Artificial Sequence
  Artificial peptide sequence
LSEKPYIV

SEQ ID NO: 546 - protein, 8 aa, Artificial Sequence
  Artificial peptide sequence
LSEKPYLL

SEQ ID NO: 547 - protein, 8 aa, Artificial Sequence
  Artificial peptide sequence
LSEKPYLI

SEQ ID NO: 548 - protein, 8 aa, Artificial Sequence
  Artificial peptide sequence
LSEKPYLV

SEQ ID NO: 549 - protein, 8 aa, Artificial Sequence
  Artificial peptide sequence
LSEKPYVL

SEQ ID NO: 550 - protein, 8 aa, Artificial Sequence
  Artificial peptide sequence
LSEKPYVI

SEQ ID NO: 551 - protein, 8 aa, Artificial Sequence
  Artificial peptide sequence
LSEKPYVV

SEQ ID NO: 552 - protein, 8 aa, Artificial Sequence
  Artificial peptide sequence
LSEKPWIL

SEQ ID NO: 553 - protein, 8 aa, Artificial Sequence
  Artificial peptide sequence
LSEKPWII

SEQ ID NO: 554 - protein, 8 aa, Artificial Sequence
  Artificial peptide sequence
LSEKPWIV

SEQ ID NO: 555 - protein, 8 aa, Artificial Sequence
  Artificial peptide sequence
LSEKPWLL

SEQ ID NO: 556 - protein, 8 aa, Artificial Sequence
  Artificial peptide sequence
LSEKPWLI

SEQ ID NO: 557 - protein, 8 aa, Artificial Sequence
  Artificial peptide sequence
LSEKPWLV

SEQ ID NO: 558 - protein, 8 aa, Artificial Sequence
  Artificial peptide sequence
LSEKPWVL

SEQ ID NO: 559 - protein, 8 aa, Artificial Sequence
  Artificial peptide sequence
LSEKPWVI

SEQ ID NO: 560 - protein, 8 aa, Artificial Sequence
  Artificial peptide sequence
LSEKPWVV

SEQ ID NO: 561 - protein, 8 aa, Artificial Sequence
  Artificial peptide sequence
LSERPYIL

SEQ ID NO: 562 - protein, 8 aa, Artificial Sequence
  Artificial peptide sequence
LSERPYII

SEQ ID NO: 563 - protein, 8 aa, Artificial Sequence
  Artificial peptide sequence
LSERPYIV

SEQ ID NO: 564 - protein, 8 aa, Artificial Sequence
  Artificial peptide sequence
LSERPYLL

SEQ ID NO: 565 - protein, 8 aa, Artificial Sequence
  Artificial peptide sequence
LSERPYLI

SEQ ID NO: 566 - protein, 8 aa, Artificial Sequence
  Artificial peptide sequence
LSERPYLV

SEQ ID NO: 567 - protein, 8 aa, Artificial Sequence
  Artificial peptide sequence
LSERPYVL

SEQ ID NO: 568 - protein, 8 aa, Artificial Sequence
  Artificial peptide sequence
LSERPYVI

SEQ ID NO: 569 - protein, 8 aa, Artificial Sequence
  Artificial peptide sequence
LSERPYVV

SEQ ID NO: 570 - protein, 8 aa, Artificial Sequence
  Artificial peptide sequence
LSERPWIL

SEQ ID NO: 571 - protein, 8 aa, Artificial Sequence
  Artificial peptide sequence
LSERPWII

SEQ ID NO: 572 - protein, 8 aa, Artificial Sequence
  Artificial peptide sequence
LSERPWIV

SEQ ID NO: 573 - protein, 8 aa, Artificial Sequence
  Artificial peptide sequence
LSERPWLL

SEQ ID NO: 574 - protein, 8 aa, Artificial Sequence
  Artificial peptide sequence
LSERPWLI

SEQ ID NO: 575 - protein, 8 aa, Artificial Sequence
  Artificial peptide sequence
LSERPWLV

SEQ ID NO: 576 - protein, 8 aa, Artificial Sequence
  Artificial peptide sequence
LSERPWVL

SEQ ID NO: 577 - protein, 8 aa, Artificial Sequence
  Artificial peptide sequence
LSERPWVI

SEQ ID NO: 578 - protein, 8 aa, Artificial Sequence
  Artificial peptide sequence
LSERPWVV

SEQ ID NO: 579 - protein, 8 aa, Artificial Sequence
  Artificial peptide sequence
LTDKPYIL

SEQ ID NO: 580 - protein, 8 aa, Artificial Sequence
  Artificial peptide sequence
LTDKPYII

SEQ ID NO: 581 - protein, 8 aa, Artificial Sequence
  Artificial peptide sequence
LTDKPYIV

SEQ ID NO: 582 - protein, 8 aa, Artificial Sequence
  Artificial peptide sequence
LTDKPYLL

SEQ ID NO: 583 - protein, 8 aa, Artificial Sequence
  Artificial peptide sequence
LTDKPYLI

SEQ ID NO: 584 - protein, 8 aa, Artificial Sequence
  Artificial peptide sequence
LTDKPYLV

SEQ ID NO: 585 - protein, 8 aa, Artificial Sequence
  Artificial peptide sequence
LTDKPYVL

SEQ ID NO: 586 - protein, 8 aa, Artificial Sequence
  Artificial peptide sequence
LTDKPYVI

SEQ ID NO: 587 - protein, 8 aa, Artificial Sequence
  Artificial peptide sequence
LTDKPYVV

SEQ ID NO: 588 - protein, 8 aa, Artificial Sequence
  Artificial peptide sequence
LTDKPWIL

SEQ ID NO: 589 - protein, 8 aa, Artificial Sequence
  Artificial peptide sequence
LTDKPWII

SEQ ID NO: 590 - protein, 8 aa, Artificial Sequence
  Artificial peptide sequence
LTDKPWIV

SEQ ID NO: 591 - protein, 8 aa, Artificial Sequence
  Artificial peptide sequence
LTDKPWLL

SEQ ID NO: 592 - protein, 8 aa, Artificial Sequence
  Artificial peptide sequence
LTDKPWLI

SEQ ID NO: 593 - protein, 8 aa, Artificial Sequence
  Artificial peptide sequence
LTDKPWLV

SEQ ID NO: 594 - protein, 8 aa, Artificial Sequence
  Artificial peptide sequence
LTDKPWVL

SEQ ID NO: 595 - protein, 8 aa, Artificial Sequence
  Artificial peptide sequence
LTDKPWVI

SEQ ID NO: 596 - protein, 8 aa, Artificial Sequence
  Artificial peptide sequence
LTDKPWVV

SEQ ID NO: 597 - protein, 8 aa, Artificial Sequence
  Artificial peptide sequence
LTDRPYIL

SEQ ID NO: 598 - protein, 8 aa, Artificial Sequence
  Artificial peptide sequence
LTDRPYII

SEQ ID NO: 599 - protein, 8 aa, Artificial Sequence
  Artificial peptide sequence
LTDRPYIV

SEQ ID NO: 600 - protein, 8 aa, Artificial Sequence
  Artificial peptide sequence
LTDRPYLL

SEQ ID NO: 601 - protein, 8 aa, Artificial Sequence
  Artificial peptide sequence
LTDRPYLI

SEQ ID NO: 602 - protein, 8 aa, Artificial Sequence
  Artificial peptide sequence
LTDRPYLV

SEQ ID NO: 603 - protein, 8 aa, Artificial Sequence
  Artificial peptide sequence
LTDRPYVL

SEQ ID NO: 604 - protein, 8 aa, Artificial Sequence
  Artificial peptide sequence
LTDRPYVI

SEQ ID NO: 605 - protein, 8 aa, Artificial Sequence
  Artificial peptide sequence
LTDRPYVV

SEQ ID NO: 606 - protein, 8 aa, Artificial Sequence
  Artificial peptide sequence
LTDRPWIL

SEQ ID NO: 607 - protein, 8 aa, Artificial Sequence
  Artificial peptide sequence
LTDRPWII

SEQ ID NO: 608 - protein, 8 aa, Artificial Sequence
  Artificial peptide sequence
LTDRPWIV

SEQ ID NO: 609 - protein, 8 aa, Artificial Sequence
  Artificial peptide sequence
LTDRPWLL

SEQ ID NO: 610 - protein, 8 aa, Artificial Sequence
  Artificial peptide sequence
LTDRPWLI

SEQ ID NO: 611 - protein, 8 aa, Artificial Sequence
  Artificial peptide sequence
LTDRPWLV

SEQ ID NO: 612 - protein, 8 aa, Artificial Sequence
  Artificial peptide sequence
LTDRPWVL

SEQ ID NO: 613 - protein, 8 aa, Artificial Sequence
  Artificial peptide sequence
LTDRPWVI

SEQ ID NO: 614 - protein, 8 aa, Artificial Sequence
  Artificial peptide sequence
LTDRPWVV

SEQ ID NO: 615 - protein, 8 aa, Artificial Sequence
  Artificial peptide sequence
LTEKPYIL

SEQ ID NO: 616 - protein, 8 aa, Artificial Sequence
  Artificial peptide sequence
LTEKPYII

SEQ ID NO: 617 - protein, 8 aa, Artificial Sequence
  Artificial peptide sequence
LTEKPYIV

SEQ ID NO: 618 - protein, 8 aa, Artificial Sequence
  Artificial peptide sequence
LTEKPYLL

SEQ ID NO: 619 - protein, 8 aa, Artificial Sequence
  Artificial peptide sequence
LTEKPYLI

SEQ ID NO: 620 - protein, 8 aa, Artificial Sequence
  Artificial peptide sequence
LTEKPYLV

SEQ ID NO: 621 - protein, 8 aa, Artificial Sequence
  Artificial peptide sequence
LTEKPYVL

SEQ ID NO: 622 - protein, 8 aa, Artificial Sequence
  Artificial peptide sequence
LTEKPYVI

SEQ ID NO: 623 - protein, 8 aa, Artificial Sequence
  Artificial peptide sequence
LTEKPYVV

SEQ ID NO: 624 - protein, 8 aa, Artificial Sequence
  Artificial peptide sequence
LTEKPWIL

SEQ ID NO: 625 - protein, 8 aa, Artificial Sequence
  Artificial peptide sequence
LTEKPWII

SEQ ID NO: 626 - protein, 8 aa, Artificial Sequence
  Artificial peptide sequence
LTEKPWIV

SEQ ID NO: 627 - protein, 8 aa, Artificial Sequence
  Artificial peptide sequence
LTEKPWLL

SEQ ID NO: 628 - protein, 8 aa, Artificial Sequence
  Artificial peptide sequence
LTEKPWLI

SEQ ID NO: 629 - protein, 8 aa, Artificial Sequence
  Artificial peptide sequence
LTEKPWLV

SEQ ID NO: 630 - protein, 8 aa, Artificial Sequence
  Artificial peptide sequence
LTEKPWVL

SEQ ID NO: 631 - protein, 8 aa, Artificial Sequence
  Artificial peptide sequence
LTEKPWVI

SEQ ID NO: 632 - protein, 8 aa, Artificial Sequence
  Artificial peptide sequence
LTEKPWVV

SEQ ID NO: 633 - protein, 8 aa, Artificial Sequence
  Artificial peptide sequence
LTERPYIL

SEQ ID NO: 634 - protein, 8 aa, Artificial Sequence
  Artificial peptide sequence
LTERPYII

SEQ ID NO: 635 - protein, 8 aa, Artificial Sequence
  Artificial peptide sequence
LTERPYIV

SEQ ID NO: 636 - protein, 8 aa, Artificial Sequence
  Artificial peptide sequence
LTERPYLL

SEQ ID NO: 637 - protein, 8 aa, Artificial Sequence
  Artificial peptide sequence
LTERPYLI

SEQ ID NO: 638 - protein, 8 aa, Artificial Sequence
  Artificial peptide sequence
LTERPYLV

SEQ ID NO: 639 - protein, 8 aa, Artificial Sequence
  Artificial peptide sequence
LTERPYVL

SEQ ID NO: 640 - protein, 8 aa, Artificial Sequence
  Artificial peptide sequence
LTERPYVI

SEQ ID NO: 641 - protein, 8 aa, Artificial Sequence
  Artificial peptide sequence
LTERPYVV

SEQ ID NO: 642 - protein, 8 aa, Artificial Sequence
  Artificial peptide sequence
LTERPWIL

SEQ ID NO: 643 - protein, 8 aa, Artificial Sequence
  Artificial peptide sequence
LTERPWII

SEQ ID NO: 644 - protein, 8 aa, Artificial Sequence
  Artificial peptide sequence
LTERPWIV

SEQ ID NO: 645 - protein, 8 aa, Artificial Sequence
  Artificial peptide sequence
LTERPWLL

SEQ ID NO: 646 - protein, 8 aa, Artificial Sequence
  Artificial peptide sequence
LTERPWLI

SEQ ID NO: 647 - protein, 8 aa, Artificial Sequence
  Artificial peptide sequence
LTERPWLV

SEQ ID NO: 648 - protein, 8 aa, Artificial Sequence
  Artificial peptide sequence
LTERPWVL

SEQ ID NO: 649 - protein, 8 aa, Artificial Sequence
  Artificial peptide sequence
LTERPWVI

SEQ ID NO: 650 - protein, 8 aa, Artificial Sequence
  Artificial peptide sequence
LTERPWVV

SEQ ID NO: 651 - protein, 8 aa, Artificial Sequence
  Artificial peptide sequence
ISDKPYIL

SEQ ID NO: 652 - protein, 8 aa, Artificial Sequence
  Artificial peptide sequence
ISDKPYII

SEQ ID NO: 653 - protein, 8 aa, Artificial Sequence
  Artificial peptide sequence
ISDKPYIV

SEQ ID NO: 654 - protein, 8 aa, Artificial Sequence
  Artificial peptide sequence
ISDKPYLL

SEQ ID NO: 655 - protein, 8 aa, Artificial Sequence
  Artificial peptide sequence
ISDKPYLI

SEQ ID NO: 656 - protein, 8 aa, Artificial Sequence
  Artificial peptide sequence
ISDKPYLV

SEQ ID NO: 657 - protein, 8 aa, Artificial Sequence
  Artificial peptide sequence
ISDKPYVL

SEQ ID NO: 658 - protein, 8 aa, Artificial Sequence
  Artificial peptide sequence
ISDKPYVI

SEQ ID NO: 659 - protein, 8 aa, Artificial Sequence
  Artificial peptide sequence
ISDKPYVV

SEQ ID NO: 660 - protein, 8 aa, Artificial Sequence
  Artificial peptide sequence
ISDKPWIL

SEQ ID NO: 661 - protein, 8 aa, Artificial Sequence
  Artificial peptide sequence
ISDKPWII

SEQ ID NO: 662 - protein, 8 aa, Artificial Sequence
  Artificial peptide sequence
ISDKPWIV

SEQ ID NO: 663 - protein, 8 aa, Artificial Sequence
  Artificial peptide sequence
ISDKPWLL

SEQ ID NO: 664 - protein, 8 aa, Artificial Sequence
  Artificial peptide sequence
ISDKPWLI

SEQ ID NO: 665 - protein, 8 aa, Artificial Sequence
  Artificial peptide sequence
ISDKPWLV

SEQ ID NO: 666 - protein, 8 aa, Artificial Sequence
  Artificial peptide sequence
ISDKPWVL

SEQ ID NO: 667 - protein, 8 aa, Artificial Sequence
  Artificial peptide sequence
ISDKPWVI

SEQ ID NO: 668 - protein, 8 aa, Artificial Sequence
  Artificial peptide sequence
ISDKPWVV

SEQ ID NO: 669 - protein, 8 aa, Artificial Sequence
  Artificial peptide sequence
ISDRPYIL

SEQ ID NO: 670 - protein, 8 aa, Artificial Sequence
  Artificial peptide sequence
ISDRPYII

SEQ ID NO: 671 - protein, 8 aa, Artificial Sequence
  Artificial peptide sequence
ISDRPYIV

SEQ ID NO: 672 - protein, 8 aa, Artificial Sequence
  Artificial peptide sequence
ISDRPYLL

SEQ ID NO: 673 - protein, 8 aa, Artificial Sequence
  Artificial peptide sequence
ISDRPYLI

SEQ ID NO: 674 - protein, 8 aa, Artificial Sequence
  Artificial peptide sequence
ISDRPYLV

SEQ ID NO: 675 - protein, 8 aa, Artificial Sequence
  Artificial peptide sequence
ISDRPYVL

SEQ ID NO: 676 - protein, 8 aa, Artificial Sequence
  Artificial peptide sequence
ISDRPYVI

SEQ ID NO: 677 - protein, 8 aa, Artificial Sequence
  Artificial peptide sequence
ISDRPYVV

SEQ ID NO: 678 - protein, 8 aa, Artificial Sequence
  Artificial peptide sequence
ISDRPWIL

SEQ ID NO: 679 - protein, 8 aa, Artificial Sequence
  Artificial peptide sequence
ISDRPWII

SEQ ID NO: 680 - protein, 8 aa, Artificial Sequence
  Artificial peptide sequence
ISDRPWIV

SEQ ID NO: 681 - protein, 8 aa, Artificial Sequence
  Artificial peptide sequence
ISDRPWLL

SEQ ID NO: 682 - protein, 8 aa, Artificial Sequence
  Artificial peptide sequence
ISDRPWLI

SEQ ID NO: 683 - protein, 8 aa, Artificial Sequence
  Artificial peptide sequence
ISDRPWLV

SEQ ID NO: 684 - protein, 8 aa, Artificial Sequence
  Artificial peptide sequence
ISDRPWVL

SEQ ID NO: 685 - protein, 8 aa, Artificial Sequence
  Artificial peptide sequence
ISDRPWVI

SEQ ID NO: 686 - protein, 8 aa, Artificial Sequence
  Artificial peptide sequence
ISDRPWVV

SEQ ID NO: 687 - protein, 8 aa, Artificial Sequence
  Artificial peptide sequence
ISEKPYIL

SEQ ID NO: 688 - protein, 8 aa, Artificial Sequence
  Artificial peptide sequence
ISEKPYII

SEQ ID NO: 689 - protein, 8 aa, Artificial Sequence
  Artificial peptide sequence
ISEKPYIV

SEQ ID NO: 690 - protein, 8 aa, Artificial Sequence
  Artificial peptide sequence
ISEKPYLL

SEQ ID NO: 691 - protein, 8 aa, Artificial Sequence
  Artificial peptide sequence
ISEKPYLI

SEQ ID NO: 692 - protein, 8 aa, Artificial Sequence
  Artificial peptide sequence
ISEKPYLV

SEQ ID NO: 693 - protein, 8 aa, Artificial Sequence
  Artificial peptide sequence
ISEKPYVL

SEQ ID NO: 694 - protein, 8 aa, Artificial Sequence
  Artificial peptide sequence
ISEKPYVI

SEQ ID NO: 695 - protein, 8 aa, Artificial Sequence
  Artificial peptide sequence
ISEKPYVV

SEQ ID NO: 696 - protein, 8 aa, Artificial Sequence
  Artificial peptide sequence
ISEKPWIL

SEQ ID NO: 697 - protein, 8 aa, Artificial Sequence
  Artificial peptide sequence
ISEKPWII

SEQ ID NO: 698 - protein, 8 aa, Artificial Sequence
  Artificial peptide sequence
ISEKPWIV

SEQ ID NO: 699 - protein, 8 aa, Artificial Sequence
  Artificial peptide sequence
ISEKPWLL

SEQ ID NO: 700 - protein, 8 aa, Artificial Sequence
  Artificial peptide sequence
ISEKPWLI

SEQ ID NO: 701 - protein, 8 aa, Artificial Sequence
  Artificial peptide sequence
ISEKPWLV

SEQ ID NO: 702 - protein, 8 aa, Artificial Sequence
  Artificial peptide sequence
ISEKPWVL

SEQ ID NO: 703 - protein, 8 aa, Artificial Sequence
  Artificial peptide sequence
ISEKPWVI

SEQ ID NO: 704 - protein, 8 aa, Artificial Sequence
  Artificial peptide sequence
ISEKPWVV

SEQ ID NO: 705 - protein, 8 aa, Artificial Sequence
  Artificial peptide sequence
ISERPYIL

SEQ ID NO: 706 - protein, 8 aa, Artificial Sequence
  Artificial peptide sequence
ISERPYII

SEQ ID NO: 707 - protein, 8 aa, Artificial Sequence
  Artificial peptide sequence
ISERPYIV

SEQ ID NO: 708 - protein, 8 aa, Artificial Sequence
  Artificial peptide sequence
ISERPYLL

SEQ ID NO: 709 - protein, 8 aa, Artificial Sequence
  Artificial peptide sequence
ISERPYLI

SEQ ID NO: 710 - protein, 8 aa, Artificial Sequence
  Artificial peptide sequence
ISERPYLV

SEQ ID NO: 711 - protein, 8 aa, Artificial Sequence
  Artificial peptide sequence
ISERPYVL

SEQ ID NO: 712 - protein, 8 aa, Artificial Sequence
  Artificial peptide sequence
ISERPYVI

SEQ ID NO: 713 - protein, 8 aa, Artificial Sequence
  Artificial peptide sequence
ISERPYVV

SEQ ID NO: 714 - protein, 8 aa, Artificial Sequence
  Artificial peptide sequence
ISERPWIL

SEQ ID NO: 715 - protein, 8 aa, Artificial Sequence
  Artificial peptide sequence
ISERPWII

SEQ ID NO: 716 - protein, 8 aa, Artificial Sequence
  Artificial peptide sequence
ISERPWIV

SEQ ID NO: 717 - protein, 8 aa, Artificial Sequence
  Artificial peptide sequence
ISERPWLL

SEQ ID NO: 718 - protein, 8 aa, Artificial Sequence
  Artificial peptide sequence
ISERPWLI

SEQ ID NO: 719 - protein, 8 aa, Artificial Sequence
  Artificial peptide sequence
ISERPWLV

SEQ ID NO: 720 - protein, 8 aa, Artificial Sequence
  Artificial peptide sequence
ISERPWVL

SEQ ID NO: 721 - protein, 8 aa, Artificial Sequence
  Artificial peptide sequence
ISERPWVI

SEQ ID NO: 722 - protein, 8 aa, Artificial Sequence
  Artificial peptide sequence
ISERPWVV

SEQ ID NO: 723 - protein, 8 aa, Artificial Sequence
  Artificial peptide sequence
ITDKPYIL

SEQ ID NO: 724 - protein, 8 aa, Artificial Sequence
  Artificial peptide sequence
ITDKPYII

SEQ ID NO: 725 - protein, 8 aa, Artificial Sequence
  Artificial peptide sequence
ITDKPYIV

SEQ ID NO: 726 - protein, 8 aa, Artificial Sequence
  Artificial peptide sequence
ITDKPYLL

SEQ ID NO: 727 - protein, 8 aa, Artificial Sequence
  Artificial peptide sequence
ITDKPYLI

SEQ ID NO: 728 - protein, 8 aa, Artificial Sequence
  Artificial peptide sequence
ITDKPYLV

SEQ ID NO: 729 - protein, 8 aa, Artificial Sequence
  Artificial peptide sequence
ITDKPYVL

SEQ ID NO: 730 - protein, 8 aa, Artificial Sequence
  Artificial peptide sequence
ITDKPYVI

SEQ ID NO: 731 - protein, 8 aa, Artificial Sequence
  Artificial peptide sequence
ITDKPYVV

SEQ ID NO: 732 - protein, 8 aa, Artificial Sequence
  Artificial peptide sequence
ITDKPWIL

SEQ ID NO: 733 - protein, 8 aa, Artificial Sequence
  Artificial peptide sequence
ITDKPWII

SEQ ID NO: 734 - protein, 8 aa, Artificial Sequence
  Artificial peptide sequence
ITDKPWIV

SEQ ID NO: 735 - protein, 8 aa, Artificial Sequence
  Artificial peptide sequence
ITDKPWLL

SEQ ID NO: 736 - protein, 8 aa, Artificial Sequence
  Artificial peptide sequence
ITDKPWLI

SEQ ID NO: 737 - protein, 8 aa, Artificial Sequence
  Artificial peptide sequence
ITDKPWLV

SEQ ID NO: 738 - protein, 8 aa, Artificial Sequence
  Artificial peptide sequence
ITDKPWVL

SEQ ID NO: 739 - protein, 8 aa, Artificial Sequence
  Artificial peptide sequence
ITDKPWVI

SEQ ID NO: 740 - protein, 8 aa, Artificial Sequence
  Artificial peptide sequence
ITDKPWVV

SEQ ID NO: 741 - protein, 8 aa, Artificial Sequence
  Artificial peptide sequence
ITDRPYIL

SEQ ID NO: 742 - protein, 8 aa, Artificial Sequence
  Artificial peptide sequence
ITDRPYII

SEQ ID NO: 743 - protein, 8 aa, Artificial Sequence
  Artificial peptide sequence
ITDRPYIV

SEQ ID NO: 744 - protein, 8 aa, Artificial Sequence
  Artificial peptide sequence
ITDRPYLL

SEQ ID NO: 745 - protein, 8 aa, Artificial Sequence
  Artificial peptide sequence
ITDRPYLI

SEQ ID NO: 746 - protein, 8 aa, Artificial Sequence
  Artificial peptide sequence
ITDRPYLV

SEQ ID NO: 747 - protein, 8 aa, Artificial Sequence
  Artificial peptide sequence
ITDRPYVL

SEQ ID NO: 748 - protein, 8 aa, Artificial Sequence
  Artificial peptide sequence
ITDRPYVI

SEQ ID NO: 749 - protein, 8 aa, Artificial Sequence
  Artificial peptide sequence
ITDRPYVV

SEQ ID NO: 750 - protein, 8 aa, Artificial Sequence
  Artificial peptide sequence
ITDRPWIL

SEQ ID NO: 751 - protein, 8 aa, Artificial Sequence
  Artificial peptide sequence
ITDRPWII

SEQ ID NO: 752 - protein, 8 aa, Artificial Sequence
  Artificial peptide sequence
ITDRPWIV

SEQ ID NO: 753 - protein, 8 aa, Artificial Sequence
  Artificial peptide sequence
ITDRPWLL

SEQ ID NO: 754 - protein, 8 aa, Artificial Sequence
  Artificial peptide sequence
ITDRPWLI

SEQ ID NO: 755 - protein, 8 aa, Artificial Sequence
  Artificial peptide sequence
ITDRPWLV

SEQ ID NO: 756 - protein, 8 aa, Artificial Sequence
  Artificial peptide sequence
ITDRPWVL

SEQ ID NO: 757 - protein, 8 aa, Artificial Sequence
  Artificial peptide sequence
ITDRPWVI

SEQ ID NO: 758 - protein, 8 aa, Artificial Sequence
  Artificial peptide sequence
ITDRPWVV

SEQ ID NO: 759 - protein, 8 aa, Artificial Sequence
  Artificial peptide sequence
ITEKPYIL

SEQ ID NO: 760 - protein, 8 aa, Artificial Sequence
  Artificial peptide sequence
ITEKPYII

SEQ ID NO: 761 - protein, 8 aa, Artificial Sequence
  Artificial peptide sequence
ITEKPYIV

SEQ ID NO: 762 - protein, 8 aa, Artificial Sequence
  Artificial peptide sequence
ITEKPYLL

SEQ ID NO: 763 - protein, 8 aa, Artificial Sequence
  Artificial peptide sequence
ITEKPYLI

SEQ ID NO: 764 - protein, 8 aa, Artificial Sequence
  Artificial peptide sequence
ITEKPYLV

SEQ ID NO: 765 - protein, 8 aa, Artificial Sequence
  Artificial peptide sequence
ITEKPYVL

SEQ ID NO: 766 - protein, 8 aa, Artificial Sequence
  Artificial peptide sequence
ITEKPYVI

SEQ ID NO: 767 - protein, 8 aa, Artificial Sequence
  Artificial peptide sequence
ITEKPYVV

SEQ ID NO: 768 - protein, 8 aa, Artificial Sequence
  Artificial peptide sequence
ITEKPWIL

SEQ ID NO: 769 - protein, 8 aa, Artificial Sequence
  Artificial peptide sequence
ITEKPWII

SEQ ID NO: 770 - protein, 8 aa, Artificial Sequence
  Artificial peptide sequence
ITEKPWIV

SEQ ID NO: 771 - protein, 8 aa, Artificial Sequence
  Artificial peptide sequence
ITEKPWLL

SEQ ID NO: 772 - protein, 8 aa, Artificial Sequence
  Artificial peptide sequence
ITEKPWLI

SEQ ID NO: 773 - protein, 8 aa, Artificial Sequence
  Artificial peptide sequence
ITEKPWLV

SEQ ID NO: 774 - protein, 8 aa, Artificial Sequence
  Artificial peptide sequence
ITEKPWVL

SEQ ID NO: 775 - protein, 8 aa, Artificial Sequence
  Artificial peptide sequence
ITEKPWVI

SEQ ID NO: 776 - protein, 8 aa, Artificial Sequence
  Artificial peptide sequence
ITEKPWVV

SEQ ID NO: 777 - protein, 8 aa, Artificial Sequence
  Artificial peptide sequence
ITERPYIL

SEQ ID NO: 778 - protein, 8 aa, Artificial Sequence
  Artificial peptide sequence
ITERPYII

SEQ ID NO: 779 - protein, 8 aa, Artificial Sequence
  Artificial peptide sequence
ITERPYIV

SEQ ID NO: 780 - protein, 8 aa, Artificial Sequence
  Artificial peptide sequence
ITERPYLL

SEQ ID NO: 781 - protein, 8 aa, Artificial Sequence
  Artificial peptide sequence
ITERPYLI

SEQ ID NO: 782 - protein, 8 aa, Artificial Sequence
  Artificial peptide sequence
ITERPYLV

SEQ ID NO: 783 - protein, 8 aa, Artificial Sequence
  Artificial peptide sequence
ITERPYVL

SEQ ID NO: 784 - protein, 8 aa, Artificial Sequence
  Artificial peptide sequence
ITERPYVI

SEQ ID NO: 785 - protein, 8 aa, Artificial Sequence
  Artificial peptide sequence
ITERPYVV

SEQ ID NO: 786 - protein, 8 aa, Artificial Sequence
  Artificial peptide sequence
ITERPWIL

SEQ ID NO: 787 - protein, 8 aa, Artificial Sequence
  Artificial peptide sequence
ITERPWII

SEQ ID NO: 788 - protein, 8 aa, Artificial Sequence
  Artificial peptide sequence
ITERPWIV

SEQ ID NO: 789 - protein, 8 aa, Artificial Sequence
  Artificial peptide sequence
ITERPWLL

SEQ ID NO: 790 - protein, 8 aa, Artificial Sequence
  Artificial peptide sequence
ITERPWLI

SEQ ID NO: 791 - protein, 8 aa, Artificial Sequence
  Artificial peptide sequence
ITERPWLV

SEQ ID NO: 792 - protein, 8 aa, Artificial Sequence
  Artificial peptide sequence
ITERPWVL

SEQ ID NO: 793 - protein, 8 aa, Artificial Sequence
  Artificial peptide sequence
ITERPWVI

SEQ ID NO: 794 - protein, 8 aa, Artificial Sequence
  Artificial peptide sequence
ITERPWVV

SEQ ID NO: 795 - protein, 8 aa, Artificial Sequence
  Artificial peptide sequence
VSDKPYIL

SEQ ID NO: 796 - protein, 8 aa, Artificial Sequence
  Artificial peptide sequence
VSDKPYII

SEQ ID NO: 797 - protein, 8 aa, Artificial Sequence
  Artificial peptide sequence
VSDKPYIV

SEQ ID NO: 798 - protein, 8 aa, Artificial Sequence
  Artificial peptide sequence
VSDKPYLL

SEQ ID NO: 799 - protein, 8 aa, Artificial Sequence
  Artificial peptide sequence
VSDKPYLI

SEQ ID NO: 800 - protein, 8 aa, Artificial Sequence
  Artificial peptide sequence
VSDKPYLV

SEQ ID NO: 801 - protein, 8 aa, Artificial Sequence
  Artificial peptide sequence
VSDKPYVL

SEQ ID NO: 802 - protein, 8 aa, Artificial Sequence
  Artificial peptide sequence
VSDKPYVI

SEQ ID NO: 803 - protein, 8 aa, Artificial Sequence
  Artificial peptide sequence
VSDKPYVV

SEQ ID NO: 804 - protein, 8 aa, Artificial Sequence
  Artificial peptide sequence
VSDKPWIL

SEQ ID NO: 805 - protein, 8 aa, Artificial Sequence
  Artificial peptide sequence
VSDKPWII

SEQ ID NO: 806 - protein, 8 aa, Artificial Sequence
  Artificial peptide sequence
VSDKPWIV

SEQ ID NO: 807 - protein, 8 aa, Artificial Sequence
  Artificial peptide sequence
VSDKPWLL

SEQ ID NO: 808 - protein, 8 aa, Artificial Sequence
  Artificial peptide sequence
VSDKPWLI

SEQ ID NO: 809 - protein, 8 aa, Artificial Sequence
  Artificial peptide sequence
VSDKPWLV

SEQ ID NO: 810 - protein, 8 aa, Artificial Sequence
  Artificial peptide sequence
VSDKPWVL

SEQ ID NO: 811 - protein, 8 aa, Artificial Sequence
  Artificial peptide sequence
VSDKPWVI

SEQ ID NO: 812 - protein, 8 aa, Artificial Sequence
  Artificial peptide sequence
VSDKPWVV

SEQ ID NO: 813 - protein, 8 aa, Artificial Sequence
  Artificial peptide sequence
VSDRPYIL

SEQ ID NO: 814 - protein, 8 aa, Artificial Sequence
  Artificial peptide sequence
VSDRPYII

SEQ ID NO: 815 - protein, 8 aa, Artificial Sequence
  Artificial peptide sequence
VSDRPYIV

SEQ ID NO: 816 - protein, 8 aa, Artificial Sequence
  Artificial peptide sequence
VSDRPYLL

SEQ ID NO: 817 - protein, 8 aa, Artificial Sequence
  Artificial peptide sequence
VSDRPYLI

SEQ ID NO: 818 - protein, 8 aa, Artificial Sequence
  Artificial peptide sequence
VSDRPYLV

SEQ ID NO: 819 - protein, 8 aa, Artificial Sequence
  Artificial peptide sequence
VSDRPYVL

SEQ ID NO: 820 - protein, 8 aa, Artificial Sequence
  Artificial peptide sequence
VSDRPYVI

SEQ ID NO: 821 - protein, 8 aa, Artificial Sequence
  Artificial peptide sequence
VSDRPYVV

SEQ ID NO: 822 - protein, 8 aa, Artificial Sequence
  Artificial peptide sequence
VSDRPWIL

SEQ ID NO: 823 - protein, 8 aa, Artificial Sequence
  Artificial peptide sequence
VSDRPWII

SEQ ID NO: 824 - protein, 8 aa, Artificial Sequence
  Artificial peptide sequence
VSDRPWIV

SEQ ID NO: 825 - protein, 8 aa, Artificial Sequence
  Artificial peptide sequence
VSDRPWLL

SEQ ID NO: 826 - protein, 8 aa, Artificial Sequence
  Artificial peptide sequence
VSDRPWLI

SEQ ID NO: 827 - protein, 8 aa, Artificial Sequence
  Artificial peptide sequence
VSDRPWLV

SEQ ID NO: 828 - protein, 8 aa, Artificial Sequence
  Artificial peptide sequence
VSDRPWVL

SEQ ID NO: 829 - protein, 8 aa, Artificial Sequence
  Artificial peptide sequence
VSDRPWVI

SEQ ID NO: 830 - protein, 8 aa, Artificial Sequence
  Artificial peptide sequence
VSDRPWVV

SEQ ID NO: 831 - protein, 8 aa, Artificial Sequence
  Artificial peptide sequence
VSEKPYIL

SEQ ID NO: 832 - protein, 8 aa, Artificial Sequence
  Artificial peptide sequence
VSEKPYII

SEQ ID NO: 833 - protein, 8 aa, Artificial Sequence
  Artificial peptide sequence
VSEKPYIV

SEQ ID NO: 834 - protein, 8 aa, Artificial Sequence
  Artificial peptide sequence
VSEKPYLL

SEQ ID NO: 835 - protein, 8 aa, Artificial Sequence
  Artificial peptide sequence
VSEKPYLI

SEQ ID NO: 836 - protein, 8 aa, Artificial Sequence
  Artificial peptide sequence
VSEKPYLV

SEQ ID NO: 837 - protein, 8 aa, Artificial Sequence
  Artificial peptide sequence
VSEKPYVL

SEQ ID NO: 838 - protein, 8 aa, Artificial Sequence
  Artificial peptide sequence
VSEKPYVI

SEQ ID NO: 839 - protein, 8 aa, Artificial Sequence
  Artificial peptide sequence
VSEKPYVV

SEQ ID NO: 840 - protein, 8 aa, Artificial Sequence
  Artificial peptide sequence
VSEKPWIL

SEQ ID NO: 841 - protein, 8 aa, Artificial Sequence
  Artificial peptide sequence
VSEKPWII

SEQ ID NO: 842 - protein, 8 aa, Artificial Sequence
  Artificial peptide sequence
VSEKPWIV

SEQ ID NO: 843 - protein, 8 aa, Artificial Sequence
  Artificial peptide sequence
VSEKPWLL

SEQ ID NO: 844 - protein, 8 aa, Artificial Sequence
  Artificial peptide sequence
VSEKPWLI

SEQ ID NO: 845 - protein, 8 aa, Artificial Sequence
  Artificial peptide sequence
VSEKPWLV

SEQ ID NO: 846 - protein, 8 aa, Artificial Sequence
  Artificial peptide sequence
VSEKPWVL

SEQ ID NO: 847 - protein, 8 aa, Artificial Sequence
  Artificial peptide sequence
VSEKPWVI

SEQ ID NO: 848 - protein, 8 aa, Artificial Sequence
  Artificial peptide sequence
VSEKPWVV

SEQ ID NO: 849 - protein, 8 aa, Artificial Sequence
  Artificial peptide sequence
VSERPYIL

SEQ ID NO: 850 - protein, 8 aa, Artificial Sequence
  Artificial peptide sequence
VSERPYII

SEQ ID NO: 851 - protein, 8 aa, Artificial Sequence
  Artificial peptide sequence
VSERPYIV

SEQ ID NO: 852 - protein, 8 aa, Artificial Sequence
  Artificial peptide sequence
VSERPYLL

SEQ ID NO: 853 - protein, 8 aa, Artificial Sequence
  Artificial peptide sequence
VSERPYLI

SEQ ID NO: 854 - protein, 8 aa, Artificial Sequence
  Artificial peptide sequence
VSERPYLV

SEQ ID NO: 855 - protein, 8 aa, Artificial Sequence
  Artificial peptide sequence
VSERPYVL

SEQ ID NO: 856 - protein, 8 aa, Artificial Sequence
  Artificial peptide sequence
VSERPYVI

SEQ ID NO: 857 - protein, 8 aa, Artificial Sequence
  Artificial peptide sequence
VSERPYVV

SEQ ID NO: 858 - protein, 8 aa, Artificial Sequence
  Artificial peptide sequence
VSERPWIL

SEQ ID NO: 859 - protein, 8 aa, Artificial Sequence
  Artificial peptide sequence
VSERPWII

SEQ ID NO: 860 - protein, 8 aa, Artificial Sequence
  Artificial peptide sequence
VSERPWIV

SEQ ID NO: 861 - protein, 8 aa, Artificial Sequence
  Artificial peptide sequence
VSERPWLL

SEQ ID NO: 862 - protein, 8 aa, Artificial Sequence
  Artificial peptide sequence
VSERPWLI

SEQ ID NO: 863 - protein, 8 aa, Artificial Sequence
  Artificial peptide sequence
VSERPWLV

SEQ ID NO: 864 - protein, 8 aa, Artificial Sequence
  Artificial peptide sequence
VSERPWVL

SEQ ID NO: 865 - protein, 8 aa, Artificial Sequence
  Artificial peptide sequence
VSERPWVI

SEQ ID NO: 866 - protein, 8 aa, Artificial Sequence
  Artificial peptide sequence
VSERPWVV

SEQ ID NO: 867 - protein, 8 aa, Artificial Sequence
  Artificial peptide sequence
VTDKPYIL

SEQ ID NO: 868 - protein, 8 aa, Artificial Sequence
  Artificial peptide sequence
VTDKPYII

SEQ ID NO: 869 - protein, 8 aa, Artificial Sequence
  Artificial peptide sequence
VTDKPYIV

SEQ ID NO: 870 - protein, 8 aa, Artificial Sequence
  Artificial peptide sequence
VTDKPYLL

SEQ ID NO: 871 - protein, 8 aa, Artificial Sequence
  Artificial peptide sequence
VTDKPYLI

SEQ ID NO: 872 - protein, 8 aa, Artificial Sequence
  Artificial peptide sequence
VTDKPYLV

SEQ ID NO: 873 - protein, 8 aa, Artificial Sequence
  Artificial peptide sequence
VTDKPYVL

SEQ ID NO: 874 - protein, 8 aa, Artificial Sequence
  Artificial peptide sequence
VTDKPYVI

SEQ ID NO: 875 - protein, 8 aa, Artificial Sequence
  Artificial peptide sequence
VTDKPYVV

SEQ ID NO: 876 - protein, 8 aa, Artificial Sequence
  Artificial peptide sequence
VTDKPWIL

SEQ ID NO: 877 - protein, 8 aa, Artificial Sequence
  Artificial peptide sequence
VTDKPWII

SEQ ID NO: 878 - protein, 8 aa, Artificial Sequence
  Artificial peptide sequence
VTDKPWIV

SEQ ID NO: 879 - protein, 8 aa, Artificial Sequence
  Artificial peptide sequence
VTDKPWLL

SEQ ID NO: 880 - protein, 8 aa, Artificial Sequence
  Artificial peptide sequence
VTDKPWLI

SEQ ID NO: 881 - protein, 8 aa, Artificial Sequence
  Artificial peptide sequence
VTDKPWLV

SEQ ID NO: 882 - protein, 8 aa, Artificial Sequence
  Artificial peptide sequence
VTDKPWVL

SEQ ID NO: 883 - protein, 8 aa, Artificial Sequence
  Artificial peptide sequence
VTDKPWVI

SEQ ID NO: 884 - protein, 8 aa, Artificial Sequence
  Artificial peptide sequence
VTDKPWVV

SEQ ID NO: 885 - protein, 8 aa, Artificial Sequence
  Artificial peptide sequence
VTDRPYIL

SEQ ID NO: 886 - protein, 8 aa, Artificial Sequence
  Artificial peptide sequence
VTDRPYII

SEQ ID NO: 887 - protein, 8 aa, Artificial Sequence
  Artificial peptide sequence
VTDRPYIV

SEQ ID NO: 888 - protein, 8 aa, Artificial Sequence
  Artificial peptide sequence
VTDRPYLL

SEQ ID NO: 889 - protein, 8 aa, Artificial Sequence
  Artificial peptide sequence
VTDRPYLI

SEQ ID NO: 890 - protein, 8 aa, Artificial Sequence
  Artificial peptide sequence
VTDRPYLV

SEQ ID NO: 891 - protein, 8 aa, Artificial Sequence
  Artificial peptide sequence
VTDRPYVL

SEQ ID NO: 892 - protein, 8 aa, Artificial Sequence
  Artificial peptide sequence
VTDRPYVI

SEQ ID NO: 893 - protein, 8 aa, Artificial Sequence
  Artificial peptide sequence
VTDRPYVV

SEQ ID NO: 894 - protein, 8 aa, Artificial Sequence
  Artificial peptide sequence
VTDRPWIL

SEQ ID NO: 895 - protein, 8 aa, Artificial Sequence
  Artificial peptide sequence
VTDRPWII

SEQ ID NO: 896 - protein, 8 aa, Artificial Sequence
  Artificial peptide sequence
VTDRPWIV

SEQ ID NO: 897 - protein, 8 aa, Artificial Sequence
  Artificial peptide sequence
VTDRPWLL

SEQ ID NO: 898 - protein, 8 aa, Artificial Sequence
  Artificial peptide sequence
VTDRPWLI

SEQ ID NO: 899 - protein, 8 aa, Artificial Sequence
  Artificial peptide sequence
VTDRPWLV

SEQ ID NO: 900 - protein, 8 aa, Artificial Sequence
  Artificial peptide sequence
VTDRPWVL

SEQ ID NO: 901 - protein, 8 aa, Artificial Sequence
  Artificial peptide sequence
VTDRPWVI

SEQ ID NO: 902 - protein, 8 aa, Artificial Sequence
  Artificial peptide sequence
VTDRPWVV

SEQ ID NO: 903 - protein, 8 aa, Artificial Sequence
  Artificial peptide sequence
VTEKPYIL

SEQ ID NO: 904 - protein, 8 aa, Artificial Sequence
  Artificial peptide sequence
VTEKPYII

SEQ ID NO: 905 - protein, 8 aa, Artificial Sequence
  Artificial peptide sequence
VTEKPYIV

SEQ ID NO: 906 - protein, 8 aa, Artificial Sequence
  Artificial peptide sequence
VTEKPYLL

SEQ ID NO: 907 - protein, 8 aa, Artificial Sequence
  Artificial peptide sequence
VTEKPYLI

SEQ ID NO: 908 - protein, 8 aa, Artificial Sequence
  Artificial peptide sequence
VTEKPYLV

SEQ ID NO: 909 - protein, 8 aa, Artificial Sequence
  Artificial peptide sequence
VTEKPYVL

SEQ ID NO: 910 - protein, 8 aa, Artificial Sequence
  Artificial peptide sequence
VTEKPYVI

SEQ ID NO: 911 - protein, 8 aa, Artificial Sequence
  Artificial peptide sequence
VTEKPYVV

SEQ ID NO: 912 - protein, 8 aa, Artificial Sequence
  Artificial peptide sequence
VTEKPWIL

SEQ ID NO: 913 - protein, 8 aa, Artificial Sequence
  Artificial peptide sequence
VTEKPWII

SEQ ID NO: 914 - protein, 8 aa, Artificial Sequence
  Artificial peptide sequence
VTEKPWIV

SEQ ID NO: 915 - protein, 8 aa, Artificial Sequence
  Artificial peptide sequence
VTEKPWLL

SEQ ID NO: 916 - protein, 8 aa, Artificial Sequence
  Artificial peptide sequence
VTEKPWLI

SEQ ID NO: 917 - protein, 8 aa, Artificial Sequence
  Artificial peptide sequence
VTEKPWLV

SEQ ID NO: 918 - protein, 8 aa, Artificial Sequence
  Artificial peptide sequence
VTEKPWVL

SEQ ID NO: 919 - protein, 8 aa, Artificial Sequence
  Artificial peptide sequence
VTEKPWVI

SEQ ID NO: 920 - protein, 8 aa, Artificial Sequence
  Artificial peptide sequence
VTEKPWVV

SEQ ID NO: 921 - protein, 8 aa, Artificial Sequence
  Artificial peptide sequence
VTERPYIL

SEQ ID NO: 922 - protein, 8 aa, Artificial Sequence
  Artificial peptide sequence
VTERPYII

SEQ ID NO: 923 - protein, 8 aa, Artificial Sequence
  Artificial peptide sequence
VTERPYIV

SEQ ID NO: 924 - protein, 8 aa, Artificial Sequence
  Artificial peptide sequence
VTERPYLL

SEQ ID NO: 925 - protein, 8 aa, Artificial Sequence
  Artificial peptide sequence
VTERPYLI

SEQ ID NO: 926 - protein, 8 aa, Artificial Sequence
  Artificial peptide sequence
VTERPYLV

SEQ ID NO: 927 - protein, 8 aa, Artificial Sequence
  Artificial peptide sequence
VTERPYVL

SEQ ID NO: 928 - protein, 8 aa, Artificial Sequence
  Artificial peptide sequence
VTERPYVI

SEQ ID NO: 929 - protein, 8 aa, Artificial Sequence
  Artificial peptide sequence
VTERPYVV

SEQ ID NO: 930 - protein, 8 aa, Artificial Sequence
  Artificial peptide sequence
VTERPWIL

SEQ ID NO: 931 - protein, 8 aa, Artificial Sequence
  Artificial peptide sequence
VTERPWII

SEQ ID NO: 932 - protein, 8 aa, Artificial Sequence
  Artificial peptide sequence
VTERPWIV

SEQ ID NO: 933 - protein, 8 aa, Artificial Sequence
  Artificial peptide sequence
VTERPWLL

SEQ ID NO: 934 - protein, 8 aa, Artificial Sequence
  Artificial peptide sequence
VTERPWLI

SEQ ID NO: 935 - protein, 8 aa, Artificial Sequence
  Artificial peptide sequence
VTERPWLV

SEQ ID NO: 936 - protein, 8 aa, Artificial Sequence
  Artificial peptide sequence
VTERPWVL

SEQ ID NO: 937 - protein, 8 aa, Artificial Sequence
  Artificial peptide sequence
VTERPWVI

SEQ ID NO: 938 - protein, 8 aa, Artificial Sequence
  Artificial peptide sequence
VTERPWVV

SEQ ID NO: 939 - protein, 8 aa, Artificial Sequence
  Artificial peptide sequence
YSDKPYIL

SEQ ID NO: 940 - protein, 8 aa, Artificial Sequence
  Artificial peptide sequence
WSDKPYIL

SEQ ID NO: 941 - protein, 8 aa, Artificial Sequence
  Artificial peptide sequence
TSDKPYIL

SEQ ID NO: 942 - protein, 8 aa, Artificial Sequence
  Artificial peptide sequence
SSDKPYIL

SEQ ID NO: 943 - protein, 8 aa, Artificial Sequence
  Artificial peptide sequence
RSDKPYIL

SEQ ID NO: 944 - protein, 8 aa, Artificial Sequence
  Artificial peptide sequence
QSDKPYIL

SEQ ID NO: 945 - protein, 8 aa, Artificial Sequence
  Artificial peptide sequence
PSDKPYIL

SEQ ID NO: 946 - protein, 8 aa, Artificial Sequence
  Artificial peptide sequence
NSDKPYIL

SEQ ID NO: 947 - protein, 8 aa, Artificial Sequence
  Artificial peptide sequence
MSDKPYIL

SEQ ID NO: 948 - protein, 8 aa, Artificial Sequence
  Artificial peptide sequence
KSDKPYIL

SEQ ID NO: 949 - protein, 8 aa, Artificial Sequence
  Artificial peptide sequence
HSDKPYIL

SEQ ID NO: 950 - protein, 8 aa, Artificial Sequence
  Artificial peptide sequence
GSDKPYIL

SEQ ID NO: 951 - protein, 8 aa, Artificial Sequence
  Artificial peptide sequence
FSDKPYIL

SEQ ID NO: 952 - protein, 8 aa, Artificial Sequence
  Artificial peptide sequence
ESDKPYIL

SEQ ID NO: 953 - protein, 8 aa, Artificial Sequence
  Artificial peptide sequence
DSDKPYIL

SEQ ID NO: 954 - protein, 8 aa, Artificial Sequence
  Artificial peptide sequence
CSDKPYIL

SEQ ID NO: 955 - protein, 7 aa, Artificial Sequence
  Artificial peptide sequence
YDKPYIL

SEQ ID NO: 956 - protein, 7 aa, Artificial Sequence
  Artificial peptide sequence
WDKPYIL

SEQ ID NO: 957 - protein, 7 aa, Artificial Sequence
  Artificial peptide sequence
VDKPYIL

SEQ ID NO: 958 - protein, 7 aa, Artificial Sequence
  Artificial peptide sequence
RDKPYIL

SEQ ID NO: 959 - protein, 7 aa, Artificial Sequence
  Artificial peptide sequence
QDKPYIL

SEQ ID NO: 960 - protein, 7 aa, Artificial Sequence
  Artificial peptide sequence
PDKPYIL

SEQ ID NO: 961 - protein, 7 aa, Artificial Sequence
  Artificial peptide sequence
NDKPYIL

SEQ ID NO: 962 - protein, 7 aa, Artificial Sequence
  Artificial peptide sequence
MDKPYIL

SEQ ID NO: 963 - protein, 7 aa, Artificial Sequence
  Artificial peptide sequence
LDKPYIL

SEQ ID NO: 964 - protein, 7 aa, Artificial Sequence
  Artificial peptide sequence
KDKPYIL

SEQ ID NO: 965 - protein, 7 aa, Artificial Sequence
  Artificial peptide sequence
IDKPYIL

SEQ ID NO: 966 - protein, 7 aa, Artificial Sequence
  Artificial peptide sequence
HDKPYIL

SEQ ID NO: 967 - protein, 7 aa, Artificial Sequence
  Artificial peptide sequence
GDKPYIL

SEQ ID NO: 968 - protein, 7 aa, Artificial Sequence
  Artificial peptide sequence
FDKPYIL

SEQ ID NO: 969 - protein, 7 aa, Artificial Sequence
  Artificial peptide sequence
EDKPYIL

SEQ ID NO: 970 - protein, 7 aa, Artificial Sequence
  Artificial peptide sequence
DDKPYIL

SEQ ID NO: 971 - protein, 7 aa, Artificial Sequence
  Artificial peptide sequence
CDKPYIL

SEQ ID NO: 972 - protein, 7 aa, Artificial Sequence
  Artificial peptide sequence
ADKPYIL

SEQ ID NO: 973 - protein, 6 aa, Artificial Sequence
  Artificial peptide sequence
YKPYIL

SEQ ID NO: 974 - protein, 6 aa, Artificial Sequence
  Artificial peptide sequence
WKPYIL

SEQ ID NO: 975 - protein, 6 aa, Artificial Sequence
  Artificial peptide sequence
VKPYIL

SEQ ID NO: 976 - protein, 6 aa, Artificial Sequence
  Artificial peptide sequence
TKPYIL

SEQ ID NO: 977 - protein, 6 aa, Artificial Sequence
  Artificial peptide sequence
SKPYIL

SEQ ID NO: 978 - protein, 6 aa, Artificial Sequence
  Artificial peptide sequence
QKPYIL

SEQ ID NO: 979 - protein, 6 aa, Artificial Sequence
  Artificial peptide sequence
PKPYIL

SEQ ID NO: 980 - protein, 6 aa, Artificial Sequence
  Artificial peptide sequence
NKPYIL

SEQ ID NO: 981 - protein, 6 aa, Artificial Sequence
  Artificial peptide sequence
MKPYIL

SEQ ID NO: 982 - protein, 6 aa, Artificial Sequence
  Artificial peptide sequence
LKPYIL

SEQ ID NO: 983 - protein, 6 aa, Artificial Sequence
  Artificial peptide sequence
IKPYIL

SEQ ID NO: 984 - protein, 6 aa, Artificial Sequence
  Artificial peptide sequence
HKPYIL

SEQ ID NO: 985 - protein, 6 aa, Artificial Sequence
  Artificial peptide sequence
FKPYIL

SEQ ID NO: 986 - protein, 6 aa, Artificial Sequence
  Artificial peptide sequence
CKPYIL

SEQ ID NO: 987 - protein, 6 aa, Artificial Sequence
  Artificial peptide sequence
AKPYIL

SEQ ID NO: 988 - protein, 5 aa, Artificial Sequence
  Artificial peptide sequence
YPYIL

SEQ ID NO: 989 - protein, 5 aa, Artificial Sequence
  Artificial peptide sequence
WPYIL

SEQ ID NO: 990 - protein, 5 aa, Artificial Sequence
  Artificial peptide sequence
VPYIL

SEQ ID NO: 991 - protein, 5 aa, Artificial Sequence
  Artificial peptide sequence
TPYIL

SEQ ID NO: 992 - protein, 5 aa, Artificial Sequence
  Artificial peptide sequence
SPYIL

SEQ ID NO: 993 - protein, 5 aa, Artificial Sequence
  Artificial peptide sequence
QPYIL

SEQ ID NO: 994 - protein, 5 aa, Artificial Sequence
  Artificial peptide sequence
PPYIL

SEQ ID NO: 995 - protein, 5 aa, Artificial Sequence
  Artificial peptide sequence
NPYIL

SEQ ID NO: 996 - protein, 5 aa, Artificial Sequence
  Artificial peptide sequence
MPYIL

SEQ ID NO: 997 - protein, 5 aa, Artificial Sequence
  Artificial peptide sequence
LPYIL

SEQ ID NO: 998 - protein, 5 aa, Artificial Sequence
  Artificial peptide sequence
IPYIL

SEQ ID NO: 999 - protein, 5 aa, Artificial Sequence
  Artificial peptide sequence
HPYIL

SEQ ID NO: 1000 - protein, 5 aa, Artificial Sequence
  Artificial peptide sequence
GPYIL

SEQ ID NO: 1001 - protein, 5 aa, Artificial Sequence
  Artificial peptide sequence
FPYIL

SEQ ID NO: 1002 - protein, 5 aa, Artificial Sequence
  Artificial peptide sequence
EPYIL

SEQ ID NO: 1003 - protein, 5 aa, Artificial Sequence
  Artificial peptide sequence
DPYIL

SEQ ID NO: 1004 - protein, 5 aa, Artificial Sequence
  Artificial peptide sequence
CPYIL

SEQ ID NO: 1005 - protein, 5 aa, Artificial Sequence
  Artificial peptide sequence
APYIL